SEQUENCE LISTING (131 sequences)

SEQ ID NO: 1 - protein, 6 aa, synthetic construct
  REGION 1..6: note = Description of Artificial Sequence: Synthetic peptide
DSAIYN

SEQ ID NO: 2 - protein, 7 aa, synthetic construct
  REGION 1..7: note = Description of Artificial Sequence: Synthetic peptide
IQSSQRE

SEQ ID NO: 3 - protein, 13 aa, synthetic construct
  REGION 1..13: note = Description of Artificial Sequence: Synthetic peptide
CAVKGSDDYKLSF

SEQ ID NO: 4 - protein, 5 aa, synthetic construct
  REGION 1..5: note = Description of Artificial Sequence: Synthetic peptide
KGHSH

SEQ ID NO: 5 - protein, 5 aa, synthetic construct
  REGION 1..5: note = Description of Artificial Sequence: Synthetic peptide
KGHSH

SEQ ID NO: 6 - protein, 19 aa, synthetic construct
  REGION 1..19: note = Description of Artificial Sequence: Synthetic peptide
CASSPVNLAGVSRADTQYF

SEQ ID NO: 7 - protein, 131 aa, synthetic construct
  REGION 1..131: note = Description of Artificial Sequence: Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKGSDDYKLSFGAGTTVTVRA

SEQ ID NO: 8 - protein, 138 aa, synthetic construct
  REGION 1..138: note = Description of Artificial Sequence: Synthetic polypeptide
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPVNLAGVSRADTQYFGPGTRLTVL

SEQ ID NO: 9 - protein, 268 aa, synthetic construct
  REGION 1..268: note = Description of Artificial Sequence: Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKGSDDYKLSFGAGTTVTVRANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 10 - protein, 311 aa, synthetic construct
  REGION 1..311: note = Description of Artificial Sequence: Synthetic polypeptide
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPVNLAGVSRADTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 11 - protein, 6 aa, synthetic construct
  REGION 1..6: note = Description of Artificial Sequence: Synthetic peptide
TSGFNG

SEQ ID NO: 12 - protein, 6 aa, synthetic construct
  REGION 1..6: note = Description of Artificial Sequence: Synthetic peptide
NVLDGL

SEQ ID NO: 13 - protein, 11 aa, synthetic construct
  REGION 1..11: note = Description of Artificial Sequence: Synthetic peptide
CAVTSWGKLQF

SEQ ID NO: 14 - protein, 5 aa, synthetic construct
  REGION 1..5: note = Description of Artificial Sequence: Synthetic peptide
SGHTA

SEQ ID NO: 15 - protein, 6 aa, synthetic construct
  REGION 1..6: note = Description of Artificial Sequence: Synthetic peptide
FQGNSA

SEQ ID NO: 16 - protein, 13 aa, synthetic construct
  REGION 1..13: note = Description of Artificial Sequence: Synthetic peptide
CASSPRGYQPQHF

SEQ ID NO: 17 - protein, 129 aa, synthetic construct
  REGION 1..129: note = Description of Artificial Sequence: Synthetic polypeptide
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVTSWGKLQFKLQFGAGTQVVVTP

SEQ ID NO: 18 - protein, 132 aa, synthetic construct
  REGION 1..132: note = Description of Artificial Sequence: Synthetic polypeptide
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSPRGYQPQHFGDGTRLSIL

SEQ ID NO: 19 - protein, 266 aa, synthetic construct
  REGION 1..266: note = Description of Artificial Sequence: Synthetic polypeptide
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVTSWGKLQFKLQFGAGTQVVVTPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 20 - protein, 305 aa, synthetic construct
  REGION 1..305: note = Description of Artificial Sequence: Synthetic polypeptide
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSPRGYQPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 21 - protein, 7 aa, synthetic construct
  REGION 1..7: note = Description of Artificial Sequence: Synthetic peptide
TSDQSYG

SEQ ID NO: 22 - protein, 8 aa, synthetic construct
  REGION 1..8: note = Description of Artificial Sequence: Synthetic peptide
QGSYDEQN

SEQ ID NO: 23 - protein, 15 aa, synthetic construct
  REGION 1..15: note = Description of Artificial Sequence: Synthetic peptide
CAMREVLDNTDKLIF

SEQ ID NO: 24 - protein, 5 aa, synthetic construct
  REGION 1..5: note = Description of Artificial Sequence: Synthetic peptide
KGHSH

SEQ ID NO: 25 - protein, 6 aa, synthetic construct
  REGION 1..6: note = Description of Artificial Sequence: Synthetic peptide
LQKENI

SEQ ID NO: 26 - protein, 16 aa, synthetic construct
  REGION 1..16: note = Description of Artificial Sequence: Synthetic peptide
CASSPPEAGLDTEAFF

SEQ ID NO: 27 - protein, 136 aa, synthetic construct
  REGION 1..136: note = Description of Artificial Sequence: Synthetic polypeptide
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREVLDNTDKLIFGTGTRLQVFP

SEQ ID NO: 28 - protein, 135 aa, synthetic construct
  REGION 1..135: note = Description of Artificial Sequence: Synthetic polypeptide
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPPEAGLDTEAFFGQGTRLTVV

SEQ ID NO: 29 - protein, 273 aa, synthetic construct
  REGION 1..273: note = Description of Artificial Sequence: Synthetic polypeptide
MSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREVLDNTDKLIFGTGTRLQVFPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 30 - protein, 308 aa, synthetic construct
  REGION 1..308: note = Description of Artificial Sequence: Synthetic polypeptide
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPPEAGLDTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 31 - protein, 6 aa, synthetic construct
  REGION 1..6: note = Description of Artificial Sequence: Synthetic peptide
NSAFQY

SEQ ID NO: 32 - protein, 6 aa, synthetic construct
  REGION 1..6: note = Description of Artificial Sequence: Synthetic peptide
TYSSGN

SEQ ID NO: 33 - protein, 13 aa, synthetic construct
  REGION 1..13: note = Description of Artificial Sequence: Synthetic peptide
CAMNSGGYQKVTF

SEQ ID NO: 34 - protein, 6 aa, synthetic construct
  REGION 1..6: note = Description of Artificial Sequence: Synthetic peptide
DFQATT

SEQ ID NO: 35 - protein, 7 aa, synthetic construct
  REGION 1..7: note = Description of Artificial Sequence: Synthetic peptide
SNEGSKA

SEQ ID NO: 36 - protein, 14 aa, synthetic construct
  REGION 1..14: note = Description of Artificial Sequence: Synthetic peptide
CSAREQGPLEEQYF

SEQ ID NO: 37 - protein, 133 aa, synthetic construct
  REGION 1..133: note = Description of Artificial Sequence: Synthetic polypeptide
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNSGGYQKVTFGIGTKLQVIP

SEQ ID NO: 38 - protein, 134 aa, synthetic construct
  REGION 1..134: note = Description of Artificial Sequence: Synthetic polypeptide
MLLLLLLLGPAGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSACSAREQGPLEEQYFGPGTRLTVT

SEQ ID NO: 39 - protein, 270 aa, synthetic construct
  REGION 1..270: note = Description of Artificial Sequence: Synthetic polypeptide
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNSGGYQKVTFGIGTKLQVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 40 - protein, 307 aa, synthetic construct
  REGION 1..307: note = Description of Artificial Sequence: Synthetic polypeptide
MLLLLLLLGPAGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSACSAREQGPLEEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 41 - protein, 7 aa, synthetic construct
  REGION 1..7: note = Description of Artificial Sequence: Synthetic peptide
TISGNEY

SEQ ID NO: 42 - protein, 5 aa, synthetic construct
  REGION 1..5: note = Description of Artificial Sequence: Synthetic peptide
GLKNN

SEQ ID NO: 43 - protein, 14 aa, synthetic construct
  REGION 1..14: note = Description of Artificial Sequence: Synthetic peptide
CIVRVAGSARQLTF

SEQ ID NO: 44 - protein, 5 aa, synthetic construct
  REGION 1..5: note = Description of Artificial Sequence: Synthetic peptide
SGHDT

SEQ ID NO: 45 - protein, 6 aa, synthetic construct
  REGION 1..6: note = Description of Artificial Sequence: Synthetic peptide
YYEEEE

SEQ ID NO: 46 - protein, 14 aa, synthetic construct
  REGION 1..14: note = Description of Artificial Sequence: Synthetic peptide
CASSFGTATYEQYF

SEQ ID NO: 47 - protein, 128 aa, synthetic construct
  REGION 1..128: note = Description of Artificial Sequence: Synthetic polypeptide
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVAGSARQLTFGSGTQLTVLP

SEQ ID NO: 48 - protein, 132 aa, synthetic construct
  REGION 1..132: note = Description of Artificial Sequence: Synthetic polypeptide
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGTATYEQYFGPGTRLTVT

SEQ ID NO: 49 - protein, 265 aa, synthetic construct
  REGION 1..265: note = Description of Artificial Sequence: Synthetic polypeptide
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVAGSARQLTFGSGTQLTVLPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 50 - protein, 305 aa, synthetic construct
  REGION 1..305: note = Description of Artificial Sequence: Synthetic polypeptide
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGTATYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 51 - protein, 6 aa, synthetic construct
  REGION 1..6: note = Description of Artificial Sequence: Synthetic peptide
DSASNY

SEQ ID NO: 52 - protein, 7 aa, synthetic construct
  REGION 1..7: note = Description of Artificial Sequence: Synthetic peptide
IRSNVGE

SEQ ID NO: 53 - protein, 14 aa, synthetic construct
  REGION 1..14: note = Description of Artificial Sequence: Synthetic peptide
CAASIPGTASKLTF

SEQ ID NO: 54 - protein, 5 aa, synthetic construct
  REGION 1..5: note = Description of Artificial Sequence: Synthetic peptide
MNHEY

SEQ ID NO: 55 - protein, 6 aa, synthetic construct
  REGION 1..6: note = Description of Artificial Sequence: Synthetic peptide
SMNVEV

SEQ ID NO: 56 - protein, 15 aa, synthetic construct
  REGION 1..15: note = Description of Artificial Sequence: Synthetic peptide
CASSPYRQGSYGYTF

SEQ ID NO: 57 - protein, 132 aa, synthetic construct
  REGION 1..132: note = Description of Artificial Sequence: Synthetic polypeptide
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASIPGTASKLTFGTGTRLQVTL

SEQ ID NO: 58 - protein, 133 aa, synthetic construct
  REGION 1..133: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPYRQGSYGYTFGSGTRLTVV

SEQ ID NO: 59 - protein, 269 aa, synthetic construct
  REGION 1..269: note = Description of Artificial Sequence: Synthetic polypeptide
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASIPGTASKLTFGTGTRLQVTLNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 60 - protein, 306 aa, synthetic construct
  REGION 1..306: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPYRQGSYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 61 - protein, 6 aa, synthetic construct
  REGION 1..6: note = Description of Artificial Sequence: Synthetic peptide
NTAFDY

SEQ ID NO: 62 - protein, 7 aa, synthetic construct
  REGION 1..7: note = Description of Artificial Sequence: Synthetic peptide
IRPDVSE

SEQ ID NO: 63 - protein, 13 aa, synthetic construct
  REGION 1..13: note = Description of Artificial Sequence: Synthetic peptide
CAASTGNFNKFYF

SEQ ID NO: 64 - protein, 5 aa, synthetic construct
  REGION 1..5: note = Description of Artificial Sequence: Synthetic peptide
SGHNS

SEQ ID NO: 65 - protein, 6 aa, synthetic construct
  REGION 1..6: note = Description of Artificial Sequence: Synthetic peptide
FNNNVP

SEQ ID NO: 66 - protein, 14 aa, synthetic construct
  REGION 1..14: note = Description of Artificial Sequence: Synthetic peptide
CASNRQGTVTEAFF

SEQ ID NO: 67 - protein, 140 aa, synthetic construct
  REGION 1..140: note = Description of Artificial Sequence: Synthetic polypeptide
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASTGNFNKFYFGSGTKLNVKP

SEQ ID NO: 68 - protein, 133 aa, synthetic construct
  REGION 1..133: note = Description of Artificial Sequence: Synthetic polypeptide
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASNRQGTVTEAFFGQGTRLTVV

SEQ ID NO: 69 - protein, 277 aa, synthetic construct
  REGION 1..277: note = Description of Artificial Sequence: Synthetic polypeptide
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASTGNFNKFYFGSGTKLNVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 70 - protein, 306 aa, synthetic construct
  REGION 1..306: note = Description of Artificial Sequence: Synthetic polypeptide
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASNRQGTVTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 71 - protein, 65 aa, synthetic construct
  REGION 1..65: note = Description of Artificial Sequence: Synthetic polypeptide
HAGLSNRLARDNELRENDKEQLKAISTRDPLSEITEQEKDFLWSHRHYCVTIPEILPKLLLSVKW

SEQ ID NO: 72 - protein, 65 aa, synthetic construct
  REGION 1..65: note = Description of Artificial Sequence: Synthetic polypeptide
HAGLSNRLARDNELRENDKEQLKAISTRDPLSKITEQEKDFLWSHRHYCVTIPEILPKLLLSVKW

SEQ ID NO: 73 - protein, 65 aa, synthetic construct
  REGION 1..65: note = Description of Artificial Sequence: Synthetic polypeptide
LSNRLARDNELRENDKEQLKAISTRDPLSEITEQEKDFLWSHRHYCVTIPEILPKLLLSVKWNSR

SEQ ID NO: 74 - protein, 65 aa, synthetic construct
  REGION 1..65: note = Description of Artificial Sequence: Synthetic polypeptide
LSNRLARDNELRENDKEQLKAISTRDPLSEITKQEKDFLWSHRHYCVTIPEILPKLLLSVKWNSR

SEQ ID NO: 75 - protein, 45 aa, synthetic construct
  REGION 1..45: note = Description of Artificial Sequence: Synthetic polypeptide
KTLALDKTEQEALEYFMKQMNDAHHGGWTTKMDWIFHTIKQHALN

SEQ ID NO: 76 - protein, 45 aa, synthetic construct
  REGION 1..45: note = Description of Artificial Sequence: Synthetic polypeptide
KTLALDKTEQEALEYFMKQMNDARHGGWTTKMDWIFHTIKQHALN

SEQ ID NO: 77 - protein, 45 aa, synthetic construct
  REGION 1..45: note = Description of Artificial Sequence: Synthetic polypeptide
KTLALDKTEQEALEYFMKQMNDALHGGWTTKMDWIFHTIKQHALN

SEQ ID NO: 78 - protein, 25 aa, synthetic construct
  REGION 1..25: note = Description of Artificial Sequence: Synthetic peptide
GHSYTTAEELAGIGILTVILGVLLL

SEQ ID NO: 79 - protein, 25 aa, synthetic construct
  REGION 1..25: note = Description of Artificial Sequence: Synthetic peptide
ILGDPKKLLTQHFVQENYLEYRQVP

SEQ ID NO: 80 - protein, 141 aa, Homo sapiens
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 81 - protein, 137 aa, synthetic construct
  REGION 1..137: note = Description of Artificial Sequence: Synthetic polypeptide
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 82 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 83 - protein, 173 aa, synthetic construct
  REGION 1..173: note = Description of Artificial Sequence: Synthetic polypeptide
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 84 - protein, 27 aa, unidentified
  REGION 1..27: note = Description of Unknown: Furin-2A-peptide sequence
RAKRSGSGATNFSLLKQAGDVEENPGP

SEQ ID NO: 85 - protein, 4 aa, synthetic construct
  REGION 1..4: note = Description of Artificial Sequence: Synthetic peptide
SGSG

SEQ ID NO: 86 - protein, 15 aa, synthetic construct
  REGION 1..15: note = Description of Artificial Sequence: Synthetic peptide
CAVRPLYGGSYIPTF

SEQ ID NO: 87 - protein, 12 aa, synthetic construct
  REGION 1..12: note = Description of Artificial Sequence: Synthetic peptide
CAVNFGGGKLIF

SEQ ID NO: 88 - protein, 13 aa, synthetic construct
  REGION 1..13: note = Description of Artificial Sequence: Synthetic peptide
CVLGGGSARQLTF

SEQ ID NO: 89 - protein, 13 aa, synthetic construct
  REGION 1..13: note = Description of Artificial Sequence: Synthetic peptide
CAVNKRSNYQLIW

SEQ ID NO: 90 - protein, 12 aa, synthetic construct
  REGION 1..12: note = Description of Artificial Sequence: Synthetic peptide
CVVNDGTYKYIF

SEQ ID NO: 91 - protein, 11 aa, synthetic construct
  REGION 1..11: note = Description of Artificial Sequence: Synthetic peptide
CAASMIARLMF

SEQ ID NO: 92 - protein, 15 aa, synthetic construct
  REGION 1..15: note = Description of Artificial Sequence: Synthetic peptide
CAMRDFSGGYNKLIF

SEQ ID NO: 93 - protein, 11 aa, synthetic construct
  REGION 1..11: note = Description of Artificial Sequence: Synthetic peptide
CAGHPLNDMRF

SEQ ID NO: 94 - protein, 13 aa, synthetic construct
  REGION 1..13: note = Description of Artificial Sequence: Synthetic peptide
CAFVRNNNARLMF

SEQ ID NO: 95 - protein, 15 aa, synthetic construct
  REGION 1..15: note = Description of Artificial Sequence: Synthetic peptide
CAESLMDTGRRALTF

SEQ ID NO: 96 - protein, 14 aa, synthetic construct
  REGION 1..14: note = Description of Artificial Sequence: Synthetic peptide
CASSYVGNTGELFF

SEQ ID NO: 97 - protein, 13 aa, synthetic construct
  REGION 1..13: note = Description of Artificial Sequence: Synthetic peptide
CASSLSFGTEAFF

SEQ ID NO: 98 - protein, 12 aa, synthetic construct
  REGION 1..12: note = Description of Artificial Sequence: Synthetic peptide
CASSRMNTEAFF

SEQ ID NO: 99 - protein, 14 aa, synthetic construct
  REGION 1..14: note = Description of Artificial Sequence: Synthetic peptide
CASSYFQGAAEAFF

SEQ ID NO: 100 - protein, 13 aa, synthetic construct
  REGION 1..13: note = Description of Artificial Sequence: Synthetic peptide
CASSPKQQYEQYF

SEQ ID NO: 101 - protein, 15 aa, synthetic construct
  REGION 1..15: note = Description of Artificial Sequence: Synthetic peptide
CASSFTTGVGTEAFF

SEQ ID NO: 102 - protein, 12 aa, synthetic construct
  REGION 1..12: note = Description of Artificial Sequence: Synthetic peptide
CASRKQNYGYTF

SEQ ID NO: 103 - protein, 14 aa, synthetic construct
  REGION 1..14: note = Description of Artificial Sequence: Synthetic peptide
CASSENGGVDEQFF

SEQ ID NO: 104 - protein, 17 aa, synthetic construct
  REGION 1..17: note = Description of Artificial Sequence: Synthetic peptide
CASSSAFTGTEDPGYTF

SEQ ID NO: 105 - protein, 15 aa, synthetic construct
  REGION 1..15: note = Description of Artificial Sequence: Synthetic peptide
CASSEWTSGDNEQFF

SEQ ID NO: 106 - protein, 15 aa, synthetic construct
  REGION 1..15: note = Description of Artificial Sequence: Synthetic peptide
CASSLGTFSTDTQYF

SEQ ID NO: 107 - protein, 15 aa, synthetic construct
  REGION 1..15: note = Description of Artificial Sequence: Synthetic peptide
CASSQGGRGEYEAFF

SEQ ID NO: 108 - protein, 11 aa, synthetic construct
  REGION 1..11: note = Description of Artificial Sequence: Synthetic peptide
CASSPGYEQFF

SEQ ID NO: 109 - protein, 20 aa, synthetic construct
  REGION 1..20: note = Description of Artificial Sequence: Synthetic peptide
CASSHPLGGQGNTWGNEQFF

SEQ ID NO: 110 - protein, 12 aa, synthetic construct
  REGION 1..12: note = Description of Artificial Sequence: Synthetic peptide
CASSFSGTGAFF

SEQ ID NO: 111 - protein, 15 aa, synthetic construct
  REGION 1..15: note = Description of Artificial Sequence: Synthetic peptide
CASSPTGTSGNEQYF

SEQ ID NO: 112 - protein, 14 aa, synthetic construct
  REGION 1..14: note = Description of Artificial Sequence: Synthetic peptide
CASSEGHLNTEAFF

SEQ ID NO: 113 - protein, 12 aa, synthetic construct
  REGION 1..12: note = Description of Artificial Sequence: Synthetic peptide
CASSRVQNEQFF

SEQ ID NO: 114 - protein, 15 aa, synthetic construct
  REGION 1..15: note = Description of Artificial Sequence: Synthetic peptide
CASSFSGTPPQPQHF

SEQ ID NO: 115 - protein, 13 aa, synthetic construct
  REGION 1..13: note = Description of Artificial Sequence: Synthetic peptide
CASSLQGYNEQFF

SEQ ID NO: 116 - protein, 13 aa, synthetic construct
  REGION 1..13: note = Description of Artificial Sequence: Synthetic peptide
CASSIPGKETQYF

SEQ ID NO: 117 - protein, 14 aa, synthetic construct
  REGION 1..14: note = Description of Artificial Sequence: Synthetic peptide
CASSTTLTGIKDTF

SEQ ID NO: 118 - protein, 13 aa, synthetic construct
  REGION 1..13: note = Description of Artificial Sequence: Synthetic peptide
CASSYGLTYEQYF

SEQ ID NO: 119 - protein, 16 aa, synthetic construct
  REGION 1..16: note = Description of Artificial Sequence: Synthetic peptide
CASSSLGDVMNTEAFF

SEQ ID NO: 120 - protein, 13 aa, synthetic construct
  REGION 1..13: note = Description of Artificial Sequence: Synthetic peptide
CASSRGLQPTQYF

SEQ ID NO: 121 - protein, 13 aa, synthetic construct
  REGION 1..13: note = Description of Artificial Sequence: Synthetic peptide
CASSFNPSYEQYF

SEQ ID NO: 122 - protein, 15 aa, synthetic construct
  REGION 1..15: note = Description of Artificial Sequence: Synthetic peptide
CASSLSGGGDYGYTF

SEQ ID NO: 123 - protein, 14 aa, synthetic construct
  REGION 1..14: note = Description of Artificial Sequence: Synthetic peptide
CASGGSGNTGKLIF

SEQ ID NO: 124 - protein, 14 aa, synthetic construct
  REGION 1..14: note = Description of Artificial Sequence: Synthetic peptide
CASSLGQFNYEQYF

SEQ ID NO: 125 - protein, 606 aa, synthetic construct
  REGION 1..606: note = Description of Artificial Sequence: Synthetic polypeptide
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPVNLAGVSRADTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKGSDDYKLSFGAGTTVTVRANIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 126 - protein, 598 aa, synthetic construct
  REGION 1..598: note = Description of Artificial Sequence: Synthetic polypeptide
MGTRLLFWVAFCLLGAYHTGAGVSQSPSNKVTEKGKDVELRCDPISGHTALYWYRQRLGQGLEFLIYFQGNSAPDKSGLPSDRFSAERTGESVSTLTIQRTQQEDSAVYLCASSPRGYQPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVTSWGKLQFKLQFGAGTQVVVTPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 127 - protein, 608 aa, synthetic construct
  REGION 1..608: note = Description of Artificial Sequence: Synthetic polypeptide
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPPEAGLDTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMSLSSLLKVVTASLWLGPGIAQKITQTQPGMFVQEKEAVTLDCTYDTSDQSYGLFWYKQPSSGEMIFLIYQGSYDEQNATEGRYSLNFQKARKSANLVISASQLGDSAMYFCAMREVLDNTDKLIFGTGTRLQVFPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 128 - protein, 604 aa, synthetic construct
  REGION 1..604: note = Description of Artificial Sequence: Synthetic polypeptide
MLLLLLLLGPAGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSACSAREQGPLEEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNSGGYQKVTFGIGTKLQVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 129 - protein, 597 aa, synthetic construct
  REGION 1..597: note = Description of Artificial Sequence: Synthetic polypeptide
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGTATYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVAGSARQLTFGSGTQLTVLPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 130 - protein, 602 aa, synthetic construct
  REGION 1..602: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPYRQGSYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAASIPGTASKLTFGTGTRLQVTLNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 131 - protein, 610 aa, synthetic construct
  REGION 1..610: note = Description of Artificial Sequence: Synthetic polypeptide
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASNRQGTVTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAASTGNFNKFYFGSGTKLNVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

(19) United States
(12) Patent Application Publication
(10) Pub. No.: US 2025/0313606 A1
(43) Pub. Date: Oct. 9, 2025

(54) T CELL RECEPTORS TARGETING PIK3CA MUTATIONS AND USES THEREOF

(71) Applicant: MEMORIAL SLOAN-KETTERING CANCER CENTER, New York, NY (US)

(72) Inventors: Christopher A. Klebanoff, New York, NY (US); Smita S. Chandran, Long Island City, NY (US)

(73) Assignee: MEMORIAL SLOAN-KETTERING CANCER CENTER, New York, NY (US)

(21) Appl. No.: 19/248,276

(22) Filed: Jun. 24, 2025

Related U.S. Application Data
(63) Continuation of application No. 17/095,288, filed on Nov. 11, 2020, now Pat. No. 12,371,467.
Application No. 17/095,288 is a continuation of application No. PCT/US2019/031749, filed on May 10, 2019.
(60) Provisional application No. 62/670,407, filed on May 11, 2018.
(60) Provisional application No. 62/688,066, filed on Jun. 21, 2018.

(51) Int. Cl. C07K 14/725; A61K 40/11; A61K 40/32; A61K 40/42; A61K 40/50

(52) U.S. Cl. CPC C07K 14/7051; A61K 40/11; A61K 40/32; A61K 40/4251; A61K 40/4269; A61K 40/4272; A61K 40/50; A61K 2239/49; C07K 2319/03

(57) ABSTRACT

The presently disclosed subject matter provides for methods and compositions for treating cancer (e.g., breast cancer). It relates to mutant PIK3CA-targeted TCRs that specifically target a mutant PIK3CA peptide (e.g., a human mutant PIK3CA peptide), and immunoresponsive cells comprising such TCRs. The presently disclosed mutant PIK3CA peptide-specific TCRs have enhanced immune-activating properties, including anti-tumor activity.

### CROSS-REFERENCE TO RELATED APPLICATIONS

This application is a Continuation of U.S. patent application Ser. No. 17/095,288, filed Nov. 11, 2020, which is a continuation of International Patent Application No. PCT/US2019/031749, filed May 10, 2019, which claims priority to U.S. Provisional Application No. 62/670,407 filed May 11, 2018, and to U.S. Provisional Application No. 62/688,066 filed Jun. 21, 2018, the contents of each of which are incorporated by reference in their entireties herein, and to each of which priority is claimed.

### SEQUENCE LISTING

The specification further incorporates by reference the Sequence Listing submitted herewith via Patent Center on Jun. 24, 2025. The Sequence Listing file, identified as 091708.0118.xml, is 178,841 bytes and was created on Jun. 23, 2025. The Sequence Listing, electronically filed herewith, does not extend beyond the scope of the specification and thus does not contain new matter.

### SEQUENCE LISTING

The specification further incorporates by reference the Sequence Listing submitted herewith via EFS on Nov. 11, 2020. Pursuant to 37 C.F.R. § 1.52 (e) (5), the Sequence Listing text file, identified as 072734_0872CON_SL.txt, is 123,243 bytes and was created on Nov. 11, 2020. The Sequence Listing, electronically filed herewith, does not extend beyond the scope of the specification and thus does not contain new matter.

### INTRODUCTION

The presently disclosed subject matter provides methods and compositions for treating cancer (e.g., breast cancer). It relates to T cell receptors (TCRs) that specifically target phosphatidylinositol-4,5-bisphosphate 3-kinase catalytic subunit alpha (PIK3CA) that comprises a mutation. The presently disclosed subject matter further provides immunoresponsive cells comprising such TCRs, and methods of using such TCRs and such cells for treating any human PIK3CA-mutated cancers, including but not limited to, breast cancer, endometrial cancer, cervical cancer, anal cancer, bladder cancer, colorectal cancer, head and neck squamous cell carcinoma, nonmelanoma skin cancer and salivary gland cancer.

### BACKGROUND OF THE INVENTION

Cell-based immunotherapy is a therapy with curative potential for the treatment of cancer. T cells and other immune cells may be modified to target tumor antigens through the introduction of genetic material coding for TCRs specific to selected antigens. Targeted T cell therapy using specific TCRs has shown recent clinical success in treating hematologic malignancies.

A third of breast cancer patients carry PIK3CA mutations, where location of hotspot mutations in PIK3CA are similar across breast cancer subtypes. Furthermore, PIK3CA has only a limited number of hotspot mutations conserved across patients in breast cancer. These features make targeting specific mutations of PIK3CA a promising strategy for targeting and eliminating breast cancer cells. Accordingly, there are needs for novel therapeutic strategies to identify and generate TCRs targeting PIK3CA comprising mutations, and for strategies capable of inducing potent cancer eradication with minimal toxicity and immunogenicity.

### SUMMARY OF THE INVENTION

The presently disclosed subject matter generally provides a T cell receptor (TCR) specifically targeting a PIK3CA peptide, wherein the PIK3CA peptide comprises a mutation. In certain embodiments, the mutation is selected from the group consisting of H104R, E545K, E542K, N345K, H1047L, E726K, C420R and any combination thereof. In certain embodiments, the mutation is H1047R or H1047L.

In certain embodiments, the TCR comprises an extracellular domain, a transmembrane domain and an intracellular domain, wherein the extracellular domain binds to the PIK3CA peptide. In certain embodiments, the extracellular domain comprises:
a) an a chain variable region CDR3 comprising the amino acid sequence set forth in SEQ ID NO: 3 or a conservative modification thereof, and a β chain variable region CDR3 comprising the amino acid sequence set forth in SEQ ID NO: 6 or a conservative modification thereof;
b) an a chain variable region CDR3 comprising the amino acid sequence set forth in SEQ ID NO: 13 or a conservative modification thereof, and a β chain variable region CDR3 comprising the amino acid sequence set forth in SEQ ID NO: 16 or a conservative modification thereof;
c) an a chain variable region CDR3 comprising the amino acid sequence set forth in SEQ ID NO: 23 or a conservative modification thereof, and a β chain variable region CDR3 comprising the amino acid sequence set forth in SEQ ID NO: 26 or a conservative modification thereof;
d) an a chain variable region CDR3 comprising the amino acid sequence set forth in SEQ ID NO: 33 or a conservative modification thereof, and a β chain variable region CDR3 comprising the amino acid sequence set forth in SEQ ID NO: 36 or a conservative modification thereof;
e) an a chain variable region CDR3 comprising the amino acid sequence set forth in SEQ ID NO: 43 or a conservative modification thereof, and a β chain variable region CDR3 comprising the amino acid sequence set forth in SEQ ID NO: 46 or a conservative modification thereof;
f) an a chain variable region CDR3 comprising the amino acid sequence set forth in SEQ ID NO: 53 or a conservative modification thereof, and a β chain variable region CDR3 comprising the amino acid sequence set forth in SEQ ID NO: 56 or a conservative modification thereof; or
g) an a chain variable region CDR3 comprising the amino acid sequence set forth in SEQ ID NO: 63 or a conservative modification thereof, and a β chain variable region CDR3 comprising the amino acid sequence set forth in SEQ ID NO: 66 or a conservative modification thereof.

In certain embodiments, the extracellular domain comprises:
a) an a chain variable region CDR2 comprising the amino acid sequence set forth in SEQ ID NO: 2 or a conservative modification thereof, and a β chain variable region CDR2 comprising the amino acid sequence set forth in SEQ ID NO: 5 or a conservative modification thereof;
b) an a chain variable region CDR2 comprising the amino acid sequence set forth in SEQ ID NO: 12 or a conservative modification thereof, and a β chain variable region CDR2 comprising the amino acid sequence set forth in SEQ ID NO: 15 or a conservative modification thereof;
c) an a chain variable region CDR2 comprising the amino acid sequence set forth in SEQ ID NO: 22 or a conservative modification thereof, and a β chain variable region CDR2 comprising the amino acid sequence set forth in SEQ ID NO: 25 or a conservative modification thereof;
d) an a chain variable region CDR2 comprising the amino acid sequence set forth in SEQ ID NO: 32 or a conservative modification thereof, and a β chain variable region CDR2 comprising the amino acid sequence set forth in SEQ ID NO: 35 or a conservative modification thereof;
e) an a chain variable region CDR2 comprising the amino acid sequence set forth in SEQ ID NO: 42 or a conservative modification thereof, and a β chain variable region CDR2 comprising the amino acid sequence set forth in SEQ ID NO: 45 or a conservative modification thereof;
f) an a chain variable region CDR2 comprising the amino acid sequence set forth in SEQ ID NO: 52 or a conservative modification thereof, and a β chain variable region CDR2 comprising the amino acid sequence set forth in SEQ ID NO: 55 or a conservative modification thereof; or
g) an a chain variable region CDR2 comprising the amino acid sequence set forth in SEQ ID NO: 62 or a conservative modification thereof, and a β chain variable region CDR2 comprising the amino acid sequence set forth in SEQ ID NO: 65 or a conservative modification thereof.

In certain embodiments, the extracellular domain comprises:
a) an a chain variable region CDR1 comprising the amino acid sequence set forth in SEQ ID NO: 1 or a conservative modification thereof, and a β chain variable region CDR1 comprising the amino acid sequence set forth in SEQ ID NO: 4 or a conservative modification thereof;
b) an a chain variable region CDR1 comprising the amino acid sequence set forth in SEQ ID NO: 11 or a conservative modification thereof, and a β chain variable region CDR1 comprising the amino acid sequence set forth in SEQ ID NO: 14 or a conservative modification thereof;
c) an a chain variable region CDR1 comprising the amino acid sequence set forth in SEQ ID NO: 21 or a conservative modification thereof, and a β chain variable region CDR1 comprising the amino acid sequence set forth in SEQ ID NO: 24 or a conservative modification thereof;
d) an a chain variable region CDR1 comprising the amino acid sequence set forth in SEQ ID NO: 31 or a conservative modification thereof, and a β chain variable region CDR1 comprising the amino acid sequence set forth in SEQ ID NO: 34 or a conservative modification thereof;
e) an a chain variable region CDR1 comprising the amino acid sequence set forth in SEQ ID NO: 41 or a conservative modification thereof, and a β chain variable region CDR1 comprising the amino acid sequence set forth in SEQ ID NO: 44 or a conservative modification thereof;
f) an a chain variable region CDR1 comprising the amino acid sequence set forth in SEQ ID NO: 51 or a conservative modification thereof, and a β chain variable region CDR1 comprising the amino acid sequence set forth in SEQ ID NO: 54 or a conservative modification thereof; or
g) an a chain variable region CDR1 comprising the amino acid sequence set forth in SEQ ID NO: 61 or a conservative modification thereof, and a β chain variable region CDR1 comprising the amino acid sequence set forth in SEQ ID NO: 64 or a conservative modification thereof.

In certain embodiments, the extracellular domain comprises:
a) an a chain variable region CDR1 comprising the amino acid sequence set forth in SEQ ID NO: 1; an a chain variable region CDR2 comprising the amino acid sequence set forth in SEQ ID NO: 2; and an a chain variable region CDR3 comprising the amino acid sequence set forth in SEQ ID NO: 3;
b) an a chain variable region CDR1 comprising the amino acid sequence set forth in SEQ ID NO: 11; an a chain variable region CDR2 comprising the amino acid sequence set forth in SEQ ID NO: 12; and an a chain variable region CDR3 comprising the amino acid sequence set forth in SEQ ID NO: 13;
c) an a chain variable region CDR1 comprising the amino acid sequence set forth in SEQ ID NO: 21; an a chain variable region CDR2 comprising the amino acid sequence set forth in SEQ ID NO: 22; and an a chain variable region CDR3 comprising the amino acid sequence set forth in SEQ ID NO: 23;
d) an a chain variable region CDR1 comprising the amino acid sequence set forth in SEQ ID NO: 31; an a chain variable region CDR2 comprising the amino acid sequence set forth in SEQ ID NO: 32; and an a chain variable region CDR3 comprising the amino acid sequence set forth in SEQ ID NO: 33;
e) an a chain variable region CDR1 comprising the amino acid sequence set forth in SEQ ID NO: 41; an a chain variable region CDR2 comprising the amino acid sequence set forth in SEQ ID NO: 42; and an a chain variable region CDR3 comprising the amino acid sequence set forth in SEQ ID NO: 43;
f) an a chain variable region CDR1 comprising the amino acid sequence set forth in SEQ ID NO: 51; an a chain variable region CDR2 comprising the amino acid sequence set forth in SEQ ID NO: 52; and an a chain variable region CDR3 comprising the amino acid sequence set forth in SEQ ID NO: 53; or
g) an a chain variable region CDR1 comprising the amino acid sequence set forth in SEQ ID NO: 61; an a chain variable region CDR2 comprising the amino acid sequence set forth in SEQ ID NO: 62; and an a chain variable region CDR3 comprising the amino acid sequence set forth in SEQ ID NO: 63.

In certain embodiments, the extracellular domain comprises:
a) a β chain variable region CDR1 comprising the amino acid sequence set forth in SEQ ID NO: 4; a β chain variable region CDR2 comprising the amino acid sequence set forth in SEQ ID NO: 5; and a β chain variable region CDR3 comprising the amino acid sequence set forth in SEQ ID NO: 6;
b) a β chain variable region CDR1 comprising the amino acid sequence set forth in SEQ ID NO: 14; a β chain variable region CDR2 comprising the amino acid sequence set forth in SEQ ID NO: 15; and a β chain variable region CDR3 comprising the amino acid sequence set forth in SEQ ID NO: 16;
c) a β chain variable region CDR1 comprising the amino acid sequence set forth in SEQ ID NO: 24; a β chain variable region CDR2 comprising the amino acid sequence set forth in SEQ ID NO: 25; and a β chain variable region CDR3 comprising the amino acid sequence set forth in SEQ ID NO: 26;
d) a β chain variable region CDR1 comprising the amino acid sequence set forth in SEQ ID NO: 34; a β chain variable region CDR2 comprising the amino acid sequence set forth in SEQ ID NO: 35; and a β chain variable region CDR3 comprising the amino acid sequence set forth in SEQ ID NO: 36;
e) a β chain variable region CDR1 comprising the amino acid sequence set forth in SEQ ID NO: 44; a β chain variable region CDR2 comprising the amino acid sequence set forth in SEQ ID NO: 45; and a β chain variable region CDR3 comprising the amino acid sequence set forth in SEQ ID NO: 46;
f) a β chain variable region CDR1 comprising the amino acid sequence set forth in SEQ ID NO: 54; a β chain variable region CDR2 comprising the amino acid sequence set forth in SEQ ID NO: 55; and a β chain variable region CDR3 comprising the amino acid sequence set forth in SEQ ID NO: 56; or
g) a β chain variable region CDR1 comprising the amino acid sequence set forth in SEQ ID NO: 64; a β chain variable region CDR2 comprising the amino acid sequence set forth in SEQ ID NO: 65; and a β chain variable region CDR3 comprising the amino acid sequence set forth in SEQ ID NO: 66.

In certain embodiments, the extracellular domain comprises:
a) an a chain variable region CDR1 comprising the amino acid sequence set forth in SEQ ID NO: 1; an a chain variable region CDR2 comprising the amino acid sequence set forth in SEQ ID NO: 2; an a chain variable region CDR3 comprising the amino acid sequence set forth in SEQ ID NO: 3; a β chain variable region CDR1 comprising the amino acid sequence set forth in SEQ ID NO: 4; a β chain variable region CDR2 comprising the amino acid sequence set forth in SEQ ID NO: 5 and a β chain variable region CDR3 comprising the amino acid sequence set forth in SEQ ID NO: 6
b) an a chain variable region CDR1 comprising the amino acid sequence set forth in SEQ ID NO: 11; an a chain variable region CDR2 comprising the amino acid sequence set forth in SEQ ID NO: 12; an a chain variable region CDR3 comprising the amino acid sequence set forth in SEQ ID NO: 13; a β chain variable region CDR1 comprising the amino acid sequence set forth in SEQ ID NO: 14; a β chain variable region CDR2 comprising the amino acid sequence set forth in SEQ ID NO: 15 and a β chain variable region CDR3 comprising the amino acid sequence set forth in SEQ ID NO: 16;
c) an a chain variable region CDR1 comprising the amino acid sequence set forth in SEQ ID NO: 21; an a chain variable region CDR2 comprising the amino acid sequence set forth in SEQ ID NO: 22; an a chain variable region CDR3 comprising the amino acid sequence set forth in SEQ ID NO: 23; a β chain variable region CDR1 comprising the amino acid sequence set forth in SEQ ID NO: 24; a β chain variable region CDR2 comprising the amino acid sequence set forth in SEQ ID NO: 25 and a β chain variable region CDR3 comprising the amino acid sequence set forth in SEQ ID NO: 26;
d) an a chain variable region CDR1 comprising the amino acid sequence set forth in SEQ ID NO: 31; an a chain variable region CDR2 comprising the amino acid sequence set forth in SEQ ID NO: 32; an a chain variable region CDR3 comprising the amino acid sequence set forth in SEQ ID NO: 33; a β chain variable region CDR1 comprising the amino acid sequence set forth in SEQ ID NO: 34; a β chain variable region CDR2 comprising the amino acid sequence set forth in SEQ ID NO: 35 and a β chain variable region CDR3 comprising the amino acid sequence set forth in SEQ ID NO: 36;
e) an a chain variable region CDR1 comprising the amino acid sequence set forth in SEQ ID NO: 41; an a chain variable region CDR2 comprising the amino acid sequence set forth in SEQ ID NO: 42; an a chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46;
f) an a chain variable region CDR1 comprising the amino acid sequence set forth in SEQ ID NO: 51; an a chain variable region CDR2 comprising the amino acid sequence set forth in SEQ ID NO: 52; an a chain variable region CDR3 comprising the amino acid sequence set forth in SEQ ID NO: 53; a β chain variable region CDR1 comprising the amino acid sequence set forth in SEQ ID NO: 54; a β chain variable region CDR2 comprising the amino acid sequence set forth in SEQ ID NO: 55 and a β chain variable region CDR3 comprising the amino acid sequence set forth in SEQ ID NO: 56; or
g) an a chain variable region CDR1 comprising the amino acid sequence set forth in SEQ ID NO: 61; an a chain variable region CDR2 comprising the amino acid sequence set forth in SEQ ID NO: 62; an a chain variable region CDR3 comprising the amino acid sequence set forth in SEQ ID NO: 63; a β chain variable region CDR1 comprising the amino acid sequence set forth in SEQ ID NO: 64; a β chain variable region CDR2 comprising the amino acid sequence set forth in SEQ ID NO: 65 and a β chain variable region CDR3 comprising the amino acid sequence set forth in SEQ ID NO: 66.

In certain embodiments, the extracellular domain comprises an a chain variable region CDR1 comprising the amino acid sequence set forth in SEQ ID NO: 41; an a chain variable region CDR2 comprising the amino acid sequence set forth in SEQ ID NO: 42; an a chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46.

In certain embodiments, the extracellular domain and comprises an a chain variable region comprising an amino acid sequence that is at least about 80% homologous or identical to the amino acid sequence set forth in SEQ ID NO: 7, SEQ ID NO: 17, SEQ ID NO: 27, SEQ ID NO: 37, SEQ ID NO: 47, SEQ ID NO: 57 or SEQ ID NO: 67.

In certain embodiments, the extracellular domain comprises an a chain variable region comprising the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67.

In certain embodiments, the extracellular domain comprises a β chain variable region comprising an amino acid sequence that is at least about 80% homologous or identical to the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68.

In certain embodiments, the extracellular domain comprises a β chain variable region comprising the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68.

In certain embodiments, the extracellular domain comprises:
a) an a chain variable region comprising an amino acid sequence that is at least about 80% homologous or identical to the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67; and
b) a β chain variable region comprising an amino acid sequence that is at least about 80% homologous or identical to the amino acid sequence set forth in SEQ ID NO: 8, SEQ ID NO: 18, SEQ ID NO: 28, SEQ ID NO: 38, SEQ ID NO: 48 SEQ ID NO: 58, or SEQ ID NO: 68.

In certain embodiments, the extracellular domain comprises: an a chain variable region comprising the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO:67; and a β chain variable region comprising the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68.

In certain embodiments, the extracellular domain comprises:
a) an a chain variable region comprising an amino acid sequence that is at least about 80% homologous or identical to the amino acid sequence set forth in SEQ ID NO: 7; and a β chain variable region comprising an amino acid sequence that is at least about 80% homologous or identical to the amino acid sequence set forth in SEQ ID NO: 8;
b) an a chain variable region comprising an amino acid sequence that is at least about 80% homologous or identical to the amino acid sequence set forth in SEQ ID NO: 17; and a β chain variable region comprising an amino acid sequence that is at least about 80% homologous or identical to the amino acid sequence set forth in SEQ ID NO: 18;
c) an a chain variable region comprising an amino acid sequence that is at least about 80% homologous or identical to the amino acid sequence set forth in SEQ ID NO: 27; and a β chain variable region comprising an amino acid sequence that is at least about 80% homologous or identical to the amino acid sequence set forth in SEQ ID NO: 28;
d) an a chain variable region comprising an amino acid sequence that is at least about 80% homologous or identical to SEQ ID NO: the amino acid sequence set forth in 37; and a β chain variable region comprising an amino acid sequence that is at least about 80% homologous or identical to the amino acid sequence set forth in SEQ ID NO: 38;
e) an a chain variable region comprising an amino acid sequence that is at least about 80% homologous or identical to the amino acid sequence set forth in SEQ ID NO: 47; and a β chain variable region comprising an amino acid sequence that is at least about 80% homologous or identical to the amino acid sequence set forth in SEQ ID NO: 48;
f) an a chain variable region comprising an amino acid sequence that is at least about 80% homologous or identical to the amino acid sequence set forth in SEQ ID NO: 57; and a β chain variable region comprising an amino acid sequence that is at least about 80% homologous or identical to the amino acid sequence set forth in SEQ ID NO: 58; or
g) an a chain variable region comprising an amino acid sequence that is at least about 80% homologous or identical to SEQ ID NO: the amino acid sequence set forth in 67; and a β chain variable region comprising an amino acid sequence that is at least about 80% homologous or identical to the amino acid sequence set forth in SEQ ID NO: 68.

In certain embodiments, the extracellular domain comprises:
a) an a chain variable region comprising the amino acid sequence set forth in SEQ ID NO: 7, and a β chain variable region comprising the amino acid sequence set forth in SEQ ID NO: 8;
b) an a chain variable region comprising the amino acid sequence set forth in SEQ ID NO: 17, and a β chain variable region comprising the amino acid sequence set forth in SEQ ID NO: 18;
c) an a chain variable region comprising the amino acid sequence set forth in SEQ ID NO: 27, and a β chain variable region comprising the amino acid sequence set forth in SEQ ID NO: 28;
d) an a chain variable region comprising the amino acid sequence set forth in SEQ ID NO: 37, and a β chain variable region comprising the amino acid sequence set forth in SEQ ID NO: 38;
e) an a chain variable region comprising the amino acid sequence set forth in SEQ ID NO: 47, and a β chain variable region comprising the amino acid sequence set forth in SEQ ID NO: 48; or
f) an a chain variable region comprising the amino acid sequence set forth in SEQ ID NO: 57, and a β chain variable region comprising the amino acid sequence set forth in SEQ ID NO: 58; or
g) an a chain variable region comprising the amino acid sequence set forth in SEQ ID NO: 67, and a β chain variable region comprising the amino acid sequence set forth in SEQ ID NO: 68.

In certain embodiments, the extracellular domain comprises an a chain variable region comprising the amino acid sequence set forth in SEQ ID NO: 47 and a β chain variable region comprising the amino acid sequence set forth in SEQ ID NO: 48.

In certain embodiments, the TCR comprises an extracellular domain comprising an a chain comprising an amino acid sequence selected from the group consisting of: SEQ ID NOS: 9, 19, 29, 39, 49, 59 and 69; and a β chain comprising an amino acid sequence selected from the group consisting of SEQ ID NOS: 10, 20, 30, 40, 50, 60 and 70. In certain embodiments, the extracellular domain comprises an a chain comprising the amino acid sequence set forth in SEQ ID NO: 49 and a β chain comprising the amino acid sequence set forth in SEQ ID NO: 50.

In certain embodiments, the extracellular domain binds to the same epitope on a human mutant PIK3CA peptide as a reference TCR or a functional fragment thereof, wherein the reference TCR or a functional fragment thereof comprises:
a) an a chain variable region CDR1 comprising the amino acid sequence set forth in SEQ ID NO: 1; an a chain variable region CDR2 comprising the amino acid sequence set forth in SEQ ID NO: 2; an a chain variable region CDR3 comprising the amino acid sequence set forth in SEQ ID NO: 3; a β chain variable region CDR1 comprising the amino acid sequence set forth in SEQ ID NO: 4; a β chain variable region CDR2 comprising the amino acid sequence set forth in SEQ ID NO: 5 and a β chain variable region CDR3 comprising the amino acid sequence set forth in SEQ ID NO: 6;
b) an a chain variable region CDR1 comprising the amino acid sequence set forth in SEQ ID NO: 11; an a chain variable region CDR2 comprising the amino acid sequence set forth in SEQ ID NO: 12; an a chain variable region CDR3 comprising the amino acid sequence set forth in SEQ ID NO: 13; a β chain variable region CDR1 comprising the amino acid sequence set forth in SEQ ID NO: 14; a β chain variable region CDR2 comprising the amino acid sequence set forth in SEQ ID NO: 15 and a β chain variable region CDR3 comprising the amino acid sequence set forth in SEQ ID NO: 16;
c) an a chain variable region CDR1 comprising the amino acid sequence set forth in SEQ ID NO: 21; an a chain variable region CDR2 comprising the amino acid sequence set forth in SEQ ID NO: 22; an a chain variable region CDR3 comprising the amino acid sequence set forth in SEQ ID NO: 23; a β chain variable region CDR1 comprising the amino acid sequence set forth in SEQ ID NO: 24; a β chain variable region CDR2 comprising the amino acid sequence set forth in SEQ ID NO: 25 and a β chain variable region CDR3 comprising the amino acid sequence set forth in SEQ ID NO: 26;
d) an a chain variable region CDR1 comprising the amino acid sequence set forth in SEQ ID NO: 31; an a chain variable region CDR2 comprising the amino acid sequence set forth in SEQ ID NO: 32; an a chain variable region CDR3 comprising the amino acid sequence set forth in SEQ ID NO: 33; a β chain variable region CDR1 comprising the amino acid sequence set forth in SEQ ID NO: 34; a β chain variable region CDR2 comprising the amino acid sequence set forth in SEQ ID NO: 35 and a β chain variable region CDR3 comprising the amino acid sequence set forth in SEQ ID NO: 36;
e) an a chain variable region CDR1 comprising the amino acid sequence set forth in SEQ ID NO: 41; an a chain variable region CDR2 comprising the amino acid sequence set forth in SEQ ID NO: 42; an a chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46;
f) an a chain variable region CDR1 comprising the amino acid sequence set forth in SEQ ID NO: 51; an a chain variable region CDR2 comprising the amino acid sequence set forth in SEQ ID NO: 52; an a chain variable region CDR3 comprising the amino acid sequence set forth in SEQ ID NO: 53; a β chain variable region CDR1 comprising the amino acid sequence set forth in SEQ ID NO: 54; a β chain variable region CDR2 comprising the amino acid sequence set forth in SEQ ID NO: 55 and a β chain variable region CDR3 comprising the amino acid sequence set forth in SEQ ID NO: 56; or
g) an a chain variable region CDR1 comprising the amino acid sequence set forth in SEQ ID NO: 61; an a chain variable region CDR2 comprising the amino acid sequence set forth in SEQ ID NO: 62; an a chain variable region CDR3 comprising the amino acid sequence set forth in SEQ ID NO: 63; a β chain variable region CDR1 comprising the amino acid sequence set forth in SEQ ID NO: 64; a β chain variable region CDR2 comprising the amino acid sequence set forth in SEQ ID NO: 65 and a β chain variable region CDR3 comprising the amino acid sequence set forth in SEQ ID NO: 66.

In certain embodiments, the reference TCR or a functional fragment thereof comprises an a chain variable region CDR1 comprising the amino acid sequence set forth in SEQ ID NO: 41; an a chain variable region CDR2 comprising the amino acid sequence set forth in SEQ ID NO: 42; an a chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46.

In certain embodiments, the TCR is recombinantly expressed, or expressed from a vector. In certain embodiments, the TCR does not target a wildtype PIK3CA peptide.

In certain embodiments, the TCR comprises a modified α-chain constant region and/or a modified β-chain constant region. In certain embodiments, the modified α-chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 80 or 81. In certain embodiments, the modified α-chain constant region comprises an amino acid sequence set forth in SEQ ID NO: 80. In certain embodiments, the modified β-chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82 or 83. In certain embodiments, the modified β-chain constant region comprises an amino acid sequence set forth in SEQ ID NO: 82.

The presently disclosed subject matter further provides an isolated immunoresponsive cell comprising the TCR disclosed herein. In certain embodiments, the immunoresponsive cell is transduced with the TCR. In certain embodiments, the TCR is constitutively expressed on the surface of the immunoresponsive cell. In certain embodiments, the immunoresponsive cell is selected from the group consisting of a T cell, a Natural Killer (NK) cell, a human embryonic stem cell, a lymphoid progenitor cell, a T cell-precursor cell, and a pluripotent stem cell from which lymphoid cells may be differentiated. In certain embodiments, the immunoresponsive cell is a T cell. In certain embodiments, the T cell is selected from the group consisting of a cytotoxic T lymphocyte (CTL), a regulatory T cell, and central memory T cells.

The presently disclosed subject matter further provides a composition comprising the immunoresponsive cell disclosed herein. In certain embodiments, the composition is a pharmaceutical composition comprising a pharmaceutically acceptable carrier.

The presently disclosed subject matter further provides an isolated nucleic acid molecule encoding the T cell receptor (TCR) disclosed herein. The presently disclosed subject matter further provides a method for producing an immunoresponsive cell that binds to a human mutant PIK3CA peptide, comprising introducing into the immunoresponsive cell a nucleic acid sequence that encodes the TCR disclosed herein.

The presently disclosed subject matter further provides a vector comprising the isolated nucleic acid molecule disclosed herein. In certain embodiments, the vector is a γ-retroviral vector.

The presently disclosed subject matter further provides a host cell comprising the nucleic acid molecule disclosed herein. In certain embodiments, the host cell is a T cell.

The presently disclosed subject matter further provides methods of treating and/or preventing a malignancy in a subject. In certain embodiments, the method comprises administering to the subject an effective amount of the immunoresponsive cell disclosed herein. In certain embodiments, the malignancy is selected from the group consisting of any PIK3CA-mutated cancers. In certain embodiments, the malignancy is selected from the group consisting of breast cancer, endometrial cancer, cervical cancer, anal cancer, bladder cancer, colorectal cancer, head and neck squamous cell carcinoma, nonmelanoma skin cancer and salivary gland cancer. In certain embodiments, the malignancy is breast cancer. In certain embodiments, the method reduces or eradicates the tumor burden in the subject. In certain embodiments, the subject is a human.

The presently disclosed subject matter further provides kits for treating and/or preventing a malignancy. In certain embodiments, the kit comprises the immunoresponsive cell disclosed herein, the isolated nucleic acid molecule disclosed herein, or the vector disclosed herein. In certain embodiments, the kit further comprises written instructions for using the immunoresponsive cell for treating a subject having a malignancy.

### BRIEF DESCRIPTION OF THE FIGURES

The following Detailed Description, given by way of example but not intended to limit the invention to specific embodiments described, may be understood in conjunction with the accompanying drawings.

FIG. 1 depicts frequency and location of PIK3CA mutations in 1,501 MSKCC HR+/HER2-breast cancer patients.

FIG. 2 depicts location of hotspot mutations in PIK3CA in breast cancer subtypes.

FIG. 3 depicts that PIK3CA had only a limited number of hotspot mutations conserved across patients in breast cancer.

FIG. 4 depicts in vitro sensitization (IVS) of donor-derived cells. PBMCs from healthy donors stimulated with autologous mature antigen-presenting cells were transfected with mRNA encoding a 65 amino acid segment of the PIK3CA gene flanking common hotspot mutations. Cells received in vitro antigen stimulation two/three times over a period of two/three weeks in the presence of low-dose IL-2 (90 IU/mL).

FIG. 5 depicts high throughput reactivity screen. Individual in vitro sensitized microwells were tested for mutation-specific reactivity by incubating with autologous APCs transfected with mRNA encoding either the wild-type (WT) or mutated PIK3CA. Mutation-specific recognition was determined by the preferential upregulation of mRNA transcript of acute inflammatory markers such as IFN-g and TNF-α by high throughput quantitative PCR.

FIG. 6 depicts isolation of mutation-specific T cells. Positive microwells underwent limiting dilution cloning to derive T cell clones of a single specificity. Individual clones were screened for mutation-specific recognition using previously described methodology; molecular sequencing was performed on positive clones to derive the alpha and beta chain sequences of its T cell receptor.

FIG. 7 depicts a demonstrative illustration of TCR reconstruction and conference of reactivity. The genetic sequences encoding the alpha and beta chains of the mutation-specific TCR were cloned into a retroviral vector and stably integrated into allogeneic PBMC. Conference of reactivity to mutation-specific transfectants and HLA-matched tumor cell line targets expressing the mutant antigen were determined by IFN-g secretion and upregulation of costimulatory molecules such as 4-1BB and OX40. A representative schema of this process is shown as an example. FIG. 7 discloses “SGSG” as SEQ ID NO: 85.

FIG. 8 depicts stimulation and screening constructs for mutant PIK3CA. FIG. 8 discloses SEQ ID NOS 71-79, respectively, in order of appearance.

FIG. 9 depicts identification of mutation-reactive microwells following in vitro sensitization. Delta CT values from sister wells against mutated or WT PI3KCA are plotted on the X and Y axis respectively. Each dot represents an individual sensitized microwell. Mutation-specific recognition of the hotspot mutations, E542K in the top panel, E545K mutation in the middle panel and the H1047R/L in the bottom panel are shown. Dots indicated by the arrows representing wells that preferentially upregulate IFN-g transcript in response to mutated PIK3CA were selected to undergo limiting dilution cloning to derive mutation-specific T cell clones.

FIG. 10 depicts screening of clones derived from limiting dilution. Growth-positive wells following limiting dilution cloning were screened for PIK3CA mutation-specific recognition by measuring the preferential upregulation of the costimulatory molecule, OX40 (left panel) and/or the release of IFN-g (right panel) in response to the mutated antigen. Positive clones were selected to undergo molecular sequencing to derive the genetic code for the alpha and beta chains of their T cell receptor.

FIG. 11 depicts high throughput screen and identification of PI3KCA-mutation reactive wells of Donor 3 T cells. Delta CT values from paired microwells against mutant or WT PIK3CA are plotted on the X and Y axis, respectively. Each dot represents an individual sensitized microwell (n=384) sensitized against one of four PI3KCA hotspot mutations listed. Well C8 (bottom left) derived from a H1047R-sensitized IVS and Well B11 (bottom right) derived from a H1047L-sensitized IVS were mutation-specific, as determined by preferential upregulation of IFN-g mRNA to mutant antigen.

FIG. 12 depicts validation of single-cell sequencing platform to correctly retrieve and quantify paired TCRa/b gene sequences from bulk populations.

FIG. 13 depicts that sequencing correctly identified, paired, and quantified known TCRs within a bulk PBMC population. FIG. 13 discloses the “CDR3: Alpha” sequences as SEQ ID NOS 86-95 and the “CDR3: Beta” sequences as SEQ ID NOS 96-121, all respectively, in order of appearance.

FIG. 14 depicts confirmation of RC8 reactivity. Autologous APCs transfected with RNA encoding either WT or the R/L substitutions at position 1047 in the PIK3CA gene were incubated with T cells from Well C8 (referred to as RC8). Delta CT values determined by upregulation of IFN-g transcript indicate mutation-specific recognition of both R and L hotspot mutations and no WT recognition. The table in the lower panel lists the CDR3 sequences (SEQ ID NOS 3, 6, 16, 6, 3, 13, 16, 26, 122, 13, 123-124, 23 and 26, respectively, in order of appearance) and frequencies of the top 10 clonotypes in RC8 derived by the platform.

FIG. 15 depicts confirmation of LB11 reactivity: Autologous APCs transfected with RNA encoding either WT or the R/L substitutions at position 1047 in the PIK3CA gene were incubated with T cells from Well B11 (referred to as LB11). Delta CT values determined by upregulation of IFN-g transcript indicate specific recognition of H1047L alone and no H1047R or WT recognition. The table in the lower panel lists the CDR3 sequences (SEQ ID NOS 36, 46, 33, 36, 43, 46, 56, 53, 56, 43, 33, 53, 63 and 66, respectively, in order of appearance) and frequencies of the top 10 clonotypes in LB11 derived by the platform.

FIG. 16 depicts the pattern of PIK3CA hotspot neoantigen reactivity and TCR repertoire was entirely distinct in T cells from well C8 and B11.

FIG. 17 depicts sequences of top clonotypes (RC8-1, RC8-2, RC8-3, LB11-1, LB11-2, LB11-3 and LB11-4) of the unique TCRs constructed into expression vectors, where the alpha chain and the beta chain of each TCR were connected by a furin-2A peptide. Based on previously described techniques, the human constant regions of the alpha and beta chains were replaced with mouse constant regions to prevent mispairing with endogenous TCR. FIG. 17 discloses SEQ ID NOS 6, 3, 125, 16, 13, 126, 26, 23, 127, 36, 33, 128, 46, 43, 129, 56, 53, 130, 66, 63 and 131, respectively, in order of appearance.

FIG. 18 is a summary of the characteristics of the PIK3CA mutation-specific TCRs.

FIG. 19 depicts confirmation of LB11 reactivity. Autologous APCs transfected with RNA encoding either WT or the R/L substitutions at position 1047 in the PIK3CA gene were incubated with T cells from Well B11 (referred to as LB11). Delta CT values determined by upregulation of IFN-g transcript indicate specific recognition of H1047L alone and no H1047R or WT recognition. Table highlights the top 4 clonotypes derived from sequencing that were selected for TCR reconstruction and testing. FIG. 19 discloses SEQ ID NOS 36, 46, 33, 36, 43, 46, 56, 53, 56, 43, 33, 53, 63 and 66, respectively, in order of appearance.

FIG. 20 depicts the efficiency of transduction of reconstructed LB11 TCRs in donor PBMCs: Allogeneic donor PBMC were transduced with retroviral particles carrying the molecular sequences for the alpha and beta chains of the LB11-derived TCR #1-4. Efficiency of transduction was determined by frequency of anti-mouse TCRβ constant staining in CD3+ (top), CD4+ (middle) and CD8+ (bottom) T cells at 4 days post-transduction.

FIG. 21 depicts confirmation of the conference of mutation-specific recognition by LB11 TCR #2. Donor PBMC with stably integrated LB11 TCR #2 were cocultured with dendritic cells expressing either H1047 WT, H1047L or H1047R mutants. Dot plots (left) and bar graph summary (right) from intracellular cytokine staining of the inflammatory markers, IFNγ, TNFα, IL-2 and CD107a show mutation-specific recognition of H1047L by transduced CD8+ cells. PMA-Ionomycin bypassing TCR-mediated stimulation was included as a positive control.

FIG. 22 depicts comparable cytokine production in a clinical grade TCR against a cancer germline antigen. Donor PBMCs were stably integrated with the cognate antigen, MAGE-A3. Dot plots (left) from intracellular cytokine staining of the inflammatory cytokines, IFNγ, TNFα, IL-2 and CD107a showed recognition of MAGE-A3 by transduced CD8+ cells. PMA-Ionomycin bypassing TCR-mediated stimulation was included as a positive control. Bar graph summary on the right comparing the cytokine-producing frequencies of mTCR+ cells in 6F9-transduced (upper) and LB11 TCR #2-transduced (lower) cells.

FIG. 23 depicts a closed loop from in vitro sensitization to TCR discovery. Healthy donor PBMCs sourced for mutation-specific TCRs allowed detection of a rare event ( 1/96). Sequencing rapidly determined the top clonotypes in the positive well. Synthetic reconstruction and testing of the TCR conclusively isolated a mutation-specific TCR, thus closing the loop from sensitization to identification to isolation of a TCR against a driver mutation. FIG. 23 discloses SEQ ID NOS 36, 46, 33, 36, 43, 46, 56, 53, 56, 43, 33, 53, 63 and 66, respectively, in order of appearance.

FIG. 24 depicts mutation-specific reactivity of TCRs identified via VDJ sequencing of well LB11. The genetic sequences encoding the alpha and beta chains of mutation-specific TCR derived from VDJ sequencing were cloned into a retroviral vector and stably integrated into allogeneic donor cells. The 3 unique TCR sequences derived from VDJ sequencing of well LB11 were tested. TCR-transduced cells were cocultured with autologous dendritic cells (DC) that had been transiently transfected with RNA encoding either wildtype or mutant PIK3CA. Reactivity was determined by observing the production of the degranulation marker, CD107A, (upper panels) or the inflammatory cytokine, TNF-α, (lower panels) in response to the DC expressing mutated PIK3CA.

FIG. 25 depicts mutation-specific reactivity of TCRs identified via VDJ sequencing of well RC8. The genetic sequences encoding the alpha and beta chains of mutation-specific TCR derived from VDJ sequencing were cloned into a retroviral vector and stably integrated into allogeneic donor cells. The 3 unique TCR sequences derived from VDJ sequencing of well RC8 were tested. TCR-transduced cells were cocultured with autologous dendritic cells (DC) that had been transiently transfected with RNA encoding either wildtype or mutant PIK3CA. Reactivity was determined by observing the production of the degranulation marker, CD107A, (upper panels) or the inflammatory cytokine, TNF-α, (lower panels) in response to the DC expressing mutated PIK3CA.

FIG. 26 depicts identification of HLA-A3 as the restriction element for PIK3CA-mutation specific TCR, LB11-2. LB11-2 TCR were retrovirally integrated into the genome of healthy donor T cells. At day 4-6 post-transduction, T cells were incubated with monkey-derived antigen-presenting cells (COS-7) that had been co-electroporated with RNA encoding a single Class I HLA allele, and either the wildtype or mutated PIK3CA RNA. The mutated version comprised a histidine to leucine substitution at position 1047 (H1047L). Cells were gated on CD8+TCR+expression. A change in production of the degranulation protein, CD107A, was used to indicate the presence of mutation-specific reactivity in the context of the right HLA allele.

FIG. 27 depicts identification of HLA-A3 as the restriction element for PIK3CA-mutation specific TCR, LB11-2. LB11-2 TCR were retrovirally integrated into the genome of healthy donor T cells. At day 4-6 post-transduction, T cells were incubated with monkey-derived antigen-presenting cells (COS-7) that had been co-electroporated with RNA encoding a single Class I HLA allele, and either the wildtype or mutated PIK3CA RNA. The mutated version comprised a histidine to leucine substitution at position 1047 (H1047L). Cells were gated on CD8+TCR+expression. A change in production of the inflammatory cytokine, TNF-α, was used to indicate the presence of mutation-specific reactivity in the context of the right HLA allele.

FIG. 28 depicts that PIK3CA-mutant specific TCR, LB11-2, is co-receptor-dependent. LB11-2 TCR were retrovirally integrated into the genome of healthy donor T cells. At day 4-6 post-transduction, T cells were incubated with monkey-derived antigen-presenting cells (COS-7) that had been co-electroporated with RNA encoding HLA-A3, and either the wildtype or mutated PIK3CA RNA. Cells were gated on CD8+ TCR+ (upper panels) or CD4+ TCR+ (lower panels) expression. A change in production of the CD107A and TNF-α, was used to indicate the presence of mutation-specific reactivity.

### DETAILED DESCRIPTION OF THE INVENTION

The presently disclosed subject matter provides TCRs targeting PIK3CA (e.g., human PIK3CA) comprising a mutation.

The presently disclosed subject matter also provides immunoresponsive cells (e.g., a T cell (e.g., a cytotoxic T lymphocyte (CTL), a regulatory T cell, a central memory T cell, etc.), a Natural Killer (NK) cell, a human embryonic stem cell, a lymphoid progenitor cell, a T cell-precursor cell, and a pluripotent stem cell from which lymphoid cells may be differentiated) comprising the PIK3CA-targeted TCRs, and methods of using such immunoresponsive cells for treating a tumor, e.g., breast cancer.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, “CDRs” are defined as the complementarity determining region amino acid sequences of a TCR, which are the hypervariable regions of TCR α-chain and β-chain. Generally, a TCR comprises at least three CDRs in the α-chain variable region and at least three CDRs in the β-chain variable region. CDRs provide the majority of contact residues for the binding of the TCR to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs are delineated using the Chothia numbering system (Chothia et al., J Mol Biol. (1987) 196:901-17). In certain embodiments, the CDRs are delineated using the AbM numbering system (Abhinandan et al., Mol. Immunol. 2008, 45, 3832-3839). In certain embodiments, the CDRs regions are delineated using the IMGT numbering system (accessible at www.imgt.org/IMGTScientificChart/Numbering/IMGTIGVLsuperfamily.html, www.imgt.org/IMGTindex/numbering.php).

Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode a TCR or a target-binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and about 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The terms “substantially homologous” or “substantially identical” mean a polypeptide or nucleic acid molecule that exhibits at least 50% homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or even about 99% homologous or identical at the amino acid level or nucleic acid to the sequence used for comparison.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e⁻³ and e⁻¹⁰⁰ indicating a closely related sequence.

As used herein, the term “analog” refers to a structurally related polypeptide or nucleic acid molecule having the function of a reference polypeptide or nucleic acid molecule.

As used herein, the term “ligand” refers to a molecule that binds to a receptor. In particular, the ligand binds a receptor on another cell, allowing for cell-to-cell recognition and/or interaction.

As used herein, the term “disease” refers to any condition or disorder that damages or interferes with the normal function of a cell, tissue, or organ. Examples of diseases include neoplasm or pathogen infection of cell.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasm), or otherwise reduce the pathological consequences of the disease (e.g., a neoplasm). The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, the term “neoplasm” refers to a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasms can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells).

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which includes or expresses a human mutant PIK3CA peptide.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment, or from whom cells are harvested).

### II. PIK3CA

Phosphatidylinositol-4,5-bisphosphate 3-kinase catalytic subunit alpha (PIK3CA; Gene ID: 5290; also known as MCM, CWS5, MCAP, PI3K, CLOVE, MCMTC, PI3K-alpha and p110-alpha), is a gene encoding a catalytic subunit of phosphatidylinositol 3 kinase, which uses ATP to phosphorylate PtdIns, PtdIns4P and PtdIns (4,5) P2. PIK3CA has been found to be oncogenic and has been implicated in breast cancer and cervical cancer.

III. T-cell receptor (TCR)

A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an a chain and a β chain (encoded by TRA and TRB, respectively). In certain embodiments, a TCR comprises a γ chain and a δ chain (encoded by TRG and TRD, respectively).

Each chain of a TCR comprises two extracellular domains: a variable region and a constant region. The constant region is proximal to the cell membrane, followed by a transmembrane domain and a short cytoplasmic tail (i.e., an intracellular domain). The variable region binds to the peptide/MHC complex. The variable region of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the presently disclosed subject matter provides a recombinant TCR. In certain embodiments, the TCR is a non-naturally occurring TCR. In certain embodiments, the TCR differs from any naturally occurring TCR by at least one amino acid residue. In certain embodiments, the TCR differs from any naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues. In certain embodiments, the TCR is modified from a naturally occurring TCR by at least one amino acid residue. In certain embodiments, the TCR is modified from a naturally occurring TCR by at least 2, 3, 4, 5, 6, 7, 8, 9, 10, 11, 12, 13, 14, 15, 20, 25, 30, 40, 50, 60, 70, 80, 90, 100 or more amino acid residues.

In certain embodiments, the TCR specifically targets a PIK3CA peptide, wherein the PIK3CA peptide comprises a mutation. In certain embodiments, the mutation is selected from the group consisting of H104R, E545K, E542K, N345K, H1047L, E726K, C420R and any combination thereof. In certain embodiments, the mutation is H1047R or H1047L. In certain embodiments, the TCR does not targets a wildtype PIK3CA peptide.

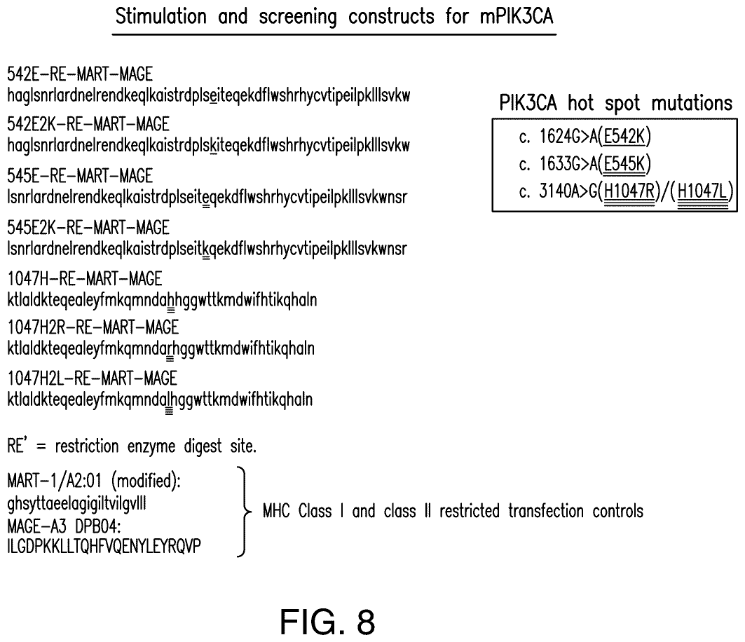

In certain embodiments, the TCR specifically targets a PIK3CA peptide listed in FIG. 8. In certain embodiments, the PIK3CA peptide comprises or has the amino acid sequence set forth in SEQ ID NO: 71, SEQ ID NO: 72, SEQ ID NO: 73, SEQ ID NO: 74, SEQ ID NO: 75, SEQ ID NO: 76, or SEQ ID NO: 77, which are provided below.

[SEQ ID NO: 71]
HAGLSNRLARDNELRENDKEQLKAISTRDPLSEITEQEKDFLWSHRHYCV
TIPEILPKLLLSVKW
[SEQ ID NO: 72]
HAGLSNRLARDNELRENDKEQLKAISTRDPLSKITEQEKDFLWSHRHYCV
TIPEILPKLLLSVKW
[SEQ ID NO: 73]
LSNRLARDNELRENDKEQLKAISTRDPLSEITEQEKDFLWSHRHYCVTIP
EILPKLLLSVKWNSR
[SEQ ID NO: 74]
LSNRLARDNELRENDKEQLKAISTRDPLSEITKQEKDFLWSHRHYCVTIP
EILPKLLLSVKWNSR
[SEQ ID NO: 75]
KTLALDKTEQEALEYFMKQMNDAHHGGWTTKMDWIFHTIKQHALN
[SEQ ID NO: 76]
KTLALDKTEQEALEYFMKQMNDARHGGWTTKMDWIFHTIKQHALN
[SEQ ID NO: 77]
KTLALDKTEQEALEYFMKQMNDALHGGWTTKMDWIFHTIKQHALN

In certain embodiments, the TCR specifically targets a PIK3CA peptide associated with a HLA class I complex, e.g., HLA-A, HLA-B and HLA-C. In certain embodiments, the TCR specifically targets a PIK3CA peptide associated with a HLA class II complex, e.g., HLA-DP, HLA-DM, HLA-DO, HLA-DQ and HLA-DR.

### TCR Clonetypes

In certain embodiments, the TCR comprises an a chain comprising the amino acid sequence set forth in SEQ ID NO: 9, SEQ ID NO: 19, SEQ ID NO: 29, SEQ ID NO: 39, SEQ ID NO: 49, SEQ ID NO: 59, or SEQ ID NO: 69. In certain embodiments, the TCR comprises a β chain comprising the amino acid sequence set forth in SEQ ID NO: 10, SEQ ID NO: 20, SEQ ID NO: 30, SEQ ID NO: 40, SEQ ID NO: 50, SEQ ID NO: 60, or SEQ ID NO: 70.

In certain embodiments, the extracellular domain of the TCR comprises an a chain variable region comprising the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67. In certain embodiments, the extracellular domain of the TCR comprises a β chain variable region comprising the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68. The sequences of SEQ ID NOS: 1-70 are described in the following Tables 1-7.

In certain embodiments, the TCR is a human TCR and specifically binds to a mutant PIK3CA peptide (e.g., a human mutant PIK3CA peptide), which is designated as RC8-1.

In certain embodiments, the TCR is a human TCR, which comprises an α chain comprising the amino acid sequence set forth in SEQ ID NO: 9 and/or a β chain comprising the amino acid sequence set forth in SEQ ID NO: 10. SEQ ID NOS: 9 and 10 are provided in Table 1.

In certain embodiments, the extracellular domain of the TCR comprises an α chain variable region and a β chain variable region or CDRs selected from Table 1. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 7, as shown in Table 1. For example, the extracellular domain comprises an α chain variable region comprising an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% homologous or identical to the amino acid sequence set forth in SEQ ID NO: 7. In certain embodiments, the extracellular domain comprises an α chain variable region comprising the amino acid sequence set forth in SEQ ID NO:7. In certain embodiments, the extracellular domain comprises a β chain variable region comprising an amino acid sequence that is at least about 80% (e.g., at least about 85%, at least about 90%, or at least about 95%) homologous or identical to the amino acid sequence set forth in SEQ ID NO: 8, as shown in Table 1. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 8. In certain embodiments, the extracellular domain comprises a β chain variable region comprising the amino acid sequence set forth in SEQ ID NO:8. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 7, and a β chain variable region comprising an amino acid sequence that is at least about 80% (e.g., at least about 85%, at least about 90%, or at least about 95%) homologous or identical to the amino acid sequence set forth in SEQ ID NO: 8. In certain embodiments, the extracellular domain comprises an α chain variable region comprising the amino acid sequence set forth in SEQ ID NO:7 and a β chain variable region comprising the amino acid sequence set forth in SEQ ID NO:8. In certain embodiments, the extracellular domain comprises an α chain variable region CDR1 comprising the amino acid sequence set forth in SEQ ID NO: 1 or a conservative modification thereof, an α chain variable region CDR2 comprising the amino acid sequence set forth in SEQ ID NO:2 or a conservative modification thereof, and an a chain variable region CDR3 comprising the amino acid sequence set forth in SEQ ID NO:3 or a conservative modification of SEQ ID NO: 3, as shown in Table 1. In certain embodiments, the extracellular domain comprises an α chain variable region CDR1 comprising the amino acid sequence set forth in SEQ ID NO:1, an α chain variable region CDR2 comprising the amino acid sequence set forth in SEQ ID NO:2, and an α chain variable region CDR3 comprising the amino acid sequence set forth in SEQ ID NO:3. In certain embodiments, the extracellular domain comprises a β chain variable region CDR1 comprising the amino acid sequence set forth in SEQ ID NO: 4 or a conservative modification thereof, a β chain variable region CDR2 comprising the amino acid sequence set forth in SEQ ID NO: 5 or a conservative modification thereof, and a β chain variable region CDR3 comprising the amino acid sequence set forth in SEQ ID NO: 6 or a conservative modification thereof, as shown in Table 1. In certain embodiments, the extracellular domain comprises a β chain variable region CDR1 comprising the amino acid sequence set forth in SEQ ID NO:4, a β chain variable region CDR2 comprising the amino acid sequence set forth in SEQ ID NO: 5, and a β chain variable region CDR3 comprising the amino acid sequence set forth in SEQ ID NO: 6. In certain embodiments, the extracellular domain comprises an α chain variable region CDR1 comprising the amino acid sequence set forth in SEQ ID NO: 1 or a conservative modification thereof, an α chain variable region CDR2 comprising the amino acid sequence set forth in SEQ ID NO: 2 or a conservative modification thereof, an α chain variable region CDR3 comprising the amino acid sequence set forth in SEQ ID NO: 3, a conservative modification of SEQ ID NO: 3, a β chain variable region CDR1 comprising the amino acid sequence set forth in SEQ ID NO: 4 or a conservative modification thereof, a β chain variable region CDR2 comprising the amino acid sequence set forth in SEQ ID NO: 5 or a conservative modification thereof, and a β chain variable region CDR3 comprising the amino acid sequence set forth in SEQ ID NO: 6 or a conservative modification thereof. In certain embodiments, the extracellular domain comprises an α chain variable region CDR1 comprising the amino acid sequence set forth in SEQ ID NO: 1, an α chain variable region CDR2 comprising the amino acid sequence set forth in SEQ ID NO: 2, an α chain variable region CDR3 comprising the amino acid sequence set forth in SEQ ID NO: 3, a β chain variable region CDR1 comprising the amino acid sequence set forth in SEQ ID NO: 4, a β chain variable region CDR2 comprising the amino acid sequence set forth in SEQ ID NO: 5, and a β chain variable region CDR3 comprising the amino acid sequence set forth in SEQ ID NO: 6.

TABLE 1
RC8-1

| Antigen | PIK3CA H1047R/L | | |
|---|---|---|---|
| CDRs | 1 | 2 | 3 |
| α-chain | DSAIYN | IQSSQRE | CAVKGSDDYKLSF |
| | [SEQ ID NO: 1] | [SEQ ID NO: 2] | [SEQ ID NO: 3] |
| β-chain | KGHSH | KGHSH | CASSPVNLAGVSRADTQYF |
| | [SEQ ID NO: 4] | [SEQ ID NO: 5] | [SEQ ID NO: 6] |

| | |
|---|---|
| α-chain | METLLGLLILWLQLQWVSSKQEVTQIPAALSVPEGENLVLNCSFTDSA |
| variable | IYNLQWFRQDPGKGLTSLLLIQSSQREQTSGRLNASLDKSSGRSTLYIA |
| | ASQPGDSATYLCAVKGSDDYKLSFGAGTTVTVRA [SEQ ID NO: 7] |
| Full α- | METLLGLLILWLQLQWVSSKQEVTQIPAALSVPEGENLVLNCSFTDSA |
| chain | IYNLQWFRQDPGKGLTSLLLIQSSQREQTSGRL |
| | NASLDKSSGRSTLYIAASQPGDSATYLCAVKGSDDYKLSFGAGTTVT |
| | VRANIQNPEPAVYQLKDPRSQDSTLCLFTDFDSQINVP |
| | KTMESGTFITDKCVLDMKAMDSKSNGAIAWSNQTSFTCQDIFKETNA |
| | TYPSSDVPCDATLTEKSFETDMNLNFQNLLVIVLRILLL |
| | KVAGFNLLMTLRLWSS [SEQ ID NO: 9] |
| β-chain | MDTRVLCCAVICLLGAGLSNAGVMQNPRHLVRRRGQEARLRCSPMK |
| variable | GHSHVYWYRQLPEEGLKFMVYLQKENIIDESGMPKERFSAEFPKEGP |
| | SILRIQQVVRGDSAAYFCASSPVNLAGVSRADTQYFGPGTRLTVL |
| | [SEQ ID NO: 8] |
| Full β- | MDTRVLCCAVICLLGAGLSNAGVMQNPRHLVRRRGQEARLRCSPMK |
| chain | GHSHVYWYRQLPEEGLKFMVYLQKENIIDESGMPKE |
| | RFSAEFPKEGPSILRIQQVVRGDSAAYFCASSPVNLAGVSRADTQYFG |
| | PGTRLTVLEDLRNVTPPKVSLFEPSKAEIANKQKATLV |
| | CLARGFFPDHVELSWWVNGKEVHSGVCTDPQAYKESNYSYCLSSRL |
| | RVSATFWHNPRNHFRCQVQFHGLSEEDKWPEGSPK |
| | PVTQNISAEAWGRADCGITSASYQQGVLSATILYEILLGKATLYAVLV |
| | STLVVMAMVKRKNS [SEQ ID NO: 10] |

In certain embodiments, the TCR is a human TCR and specifically binds to a mutant PIK3CA peptide (e.g., a human mutant PIK3CA peptide), which is designated as “RC8-2”.

In certain embodiments, the TCR is a human TCR, which comprises an α chain comprising the amino acid sequence set forth in SEQ ID NO: 19 and/or a β chain comprising the amino acid sequence set forth in SEQ ID NO: 20. SEQ ID NOS: 19 and 20 are provided in Table 2.

In certain embodiments, the extracellular domain of the TCR comprises an α chain variable region and a β chain variable region or CDRs selected from Table 2. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 17, as shown in Table 2. For example, the extracellular domain comprises an α chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 17. In certain embodiments, the extracellular domain comprises an α chain variable region comprising the amino acid sequence set forth in SEQ ID NO: 17. In certain embodiments, the extracellular domain comprises a β chain variable region comprising an amino acid sequence that is at least about 80% (e.g., at least about 85%, at least about 90%, or at least about 95%) homologous or identical to SEQ ID NO: 18, as shown in Table 2. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 18. In certain embodiments, the extracellular domain comprises a β chain variable region comprising the amino acid sequence set forth in SEQ ID NO: 18. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 17, and a β chain variable region comprising an amino acid sequence that is at least about 80% (e.g., at least about 85%, at least about 90%, or at least about 95%) homologous or identical to the amino acid sequence set forth in SEQ ID NO: 18. In certain embodiments, the extracellular domain comprises an α chain variable region comprising the amino acid sequence set forth in SEQ ID NO: 17 and a β chain variable region comprising the amino acid sequence set forth in SEQ ID NO: 18. In certain embodiments, the extracellular domain comprises an α chain variable region CDR1 comprising the amino acid sequence set forth in SEQ ID NO: 11 or a conservative modification thereof, an α chain variable region CDR2 comprising the amino acid sequence set forth in SEQ ID NO: 12 or a conservative modification thereof, and an α chain variable region CDR3 comprising the amino acid sequence set forth in SEQ ID NO: 13 or a conservative modification thereof, as shown in Table 2. In certain embodiments, the extracellular domain comprises an α chain variable region CDR1 comprising the amino acid sequence set forth in SEQ ID NO: 11, an α chain variable region CDR2 comprising the amino acid sequence set forth in SEQ ID NO: 12, and an α chain variable region CDR3 comprising the amino acid sequence set forth in SEQ ID NO: 13. In certain embodiments, the extracellular domain comprises a β chain variable region CDR1 comprising the amino acid sequence set forth in SEQ ID NO: 14 or a conservative modification thereof, a β chain variable region CDR2 comprising the amino acid sequence set forth in SEQ ID NO: 15 or a conservative modification thereof, and a β chain variable region CDR3 comprising the amino acid sequence set forth in SEQ ID NO: 16 or a conservative modification thereof, as shown in Table 2. In certain embodiments, the extracellular domain comprises a β chain variable region CDR1 comprising the amino acid sequence set forth in SEQ ID NO: 14, a β chain variable region CDR2 comprising the amino acid sequence set forth in SEQ ID NO: 15, and a β chain variable region CDR3 comprising the amino acid sequence set forth in SEQ ID NO: 16. In certain embodiments, the extracellular domain comprises an α chain variable region CDR1 comprising the amino acid sequence set forth in SEQ ID NO: 11 or a conservative modification thereof, an α chain variable region CDR2 comprising the amino acid sequence set forth in SEQ ID NO: 12 or a conservative modification thereof, an α chain variable region CDR3 comprising the amino acid sequence set forth in SEQ ID NO: 13 or a conservative modification thereof, a β chain variable region CDR1 comprising the amino acid sequence set forth in SEQ ID NO: 14 or a conservative modification thereof, a β chain variable region CDR2 comprising the amino acid sequence set forth in SEQ ID NO: 15 or a conservative modification thereof, and a β chain variable region CDR3 comprising the amino acid sequence set forth in SEQ ID NO: 16 or a conservative modification thereof. In certain embodiments, the extracellular domain comprises an α chain variable region CDR1 comprising the amino acid sequence set forth in SEQ ID NO: 11, an α chain variable region CDR2 comprising the amino acid sequence set forth in SEQ ID NO: 12, an α chain variable region CDR3 comprising the amino acid sequence set forth in SEQ ID NO: 13, a β chain variable region CDR1 comprising the amino acid sequence set forth in SEQ ID NO: 14, a β chain variable region CDR2 comprising the amino acid sequence set forth in SEQ ID NO: 15, and a β chain variable region CDR3 comprising the amino acid sequence set forth in SEQ ID NO: 16.

TABLE 2
RC8-2

| Antigen | PIK3CA H1047R/L | | |
|---|---|---|---|
| CDRs | 1 | 2 | 3 |
| α-chain | TSGFNG | NVLDGL | CAVTSWGKLQF |
| | [SEQ ID NO: 11] | [SEQ ID NO: 12] | [SEQ ID NO: 13] |
| β-chain | SGHTA | FQGNSA | CASSPRGYQPQHF |
| | [SEQ ID NO: 14] | [SEQ ID NO: 15] | [SEQ ID NO: 16] |

| | |
|---|---|
| α-chain | MWGVFLLYVSMKMGGTTGQNIDQPTEMTATEGAIVQINCTYQTSGFN |
| variable | GLFWYQQHAGEAPTFLSYNVLDGLEEKGRFSSFLSRSKGYSYLLLKEL |
| | QMKDSASYLCAVTSWGKLQFKLQFGAGTQVVVTP[SEQ ID NO: 17] |
| Full α- | MWGVFLLYVSMKMGGTTGQNIDQPTEMTATEGAIVQINCTYQTSGFN |
| chain | GLFWYQQHAGEAPTFLSYNVLDGLEEKGRFSSFLSRSK |
| | GYSYLLLKELQMKDSASYLCAVTSWGKLQFKLQFGAGTQVVVTPNIQ |
| | NPEPAVYQLKDPRSQDSTLCLFTDFDSQINVPKTMES |
| | GTFITDKCVLDMKAMDSKSNGAIAWSNQTSFTCQDIFKETNATYPSSD |
| | VPCDATLTEKSFETDMNLNFQNLLVIVLRILLLKVAGFN |
| | LLMTLRLWSS |
| | [SEQ ID NO: 19] |
| β-chain | MGTRLLFWVAFCLLGAYHTGAGVSQSPSNKVTEKGKDVELRCDPISGH |
| variable | TALYWYRQRLGQGLEFLIYFQGNSAPDKSGLPSDRFSAERTGESVSTLTI |
| | QRTQQEDSAVYLCASSPRGYQPQHFGDGTRLSIL [SEQ ID NO: 18] |
| Full β- | MGTRLLFWVAFCLLGAYHTGAGVSQSPSNKVTEKGKDVELRCDPISGH |
| chain | TALYWYRQRLGQGLEFLIYFQGNSAPDKSGLPSDR |
| | FSAERTGESVSTLTIQRTQQEDSAVYLCASSPRGYQPQHFGDGTRLSILE |
| | DLRNVTPPKVSLFEPSKAEIANKQKATLVCLARGFF |
| | PDHVELSWWVNGKEVHSGVCTDPQAYKESNYSYCLSSRLRVSATFWH |
| | NPRNHFRCQVQFHGLSEEDKWPEGSPKPVTQNIS |
| | AEAWGRADCGITSASYQQGVLSATILYEILLGKATLYAVLVSTLVVMA |
| | MVKRKNS [SEQ ID NO: 20] |

In certain embodiments, the TCR is a human TCR and specifically binds to a mutant PIK3CA peptide polypeptide (e.g., a human mutant PIK3CA peptide polypeptide), which is designated as RC8-3.

In certain embodiments, the TCR is a human TCR, which comprises an α chain comprising the amino acid sequence set forth in SEQ ID NO: 29 and/or a β chain comprising the amino acid sequence set forth in SEQ ID NO: 30. SEQ ID NOS: 29 and 30 are provided in Table 3.

In certain embodiments, the extracellular domain of the TCR comprises an α chain variable region and a β chain variable region or CDRs selected from Table 3. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 27, as shown in Table 3. For example, the extracellular domain comprises an α chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 27. In certain embodiments, the extracellular domain comprises an α chain variable region comprising the amino acid sequence set forth in SEQ ID NO: 27. In certain embodiments, the extracellular domain comprises a β chain variable region comprising an amino acid sequence that is at least about 80% (e.g., at least about 85%, at least about 90%, or at least about 95%) homologous or identical to the amino acid sequence set forth in SEQ ID NO: 28, as shown in Table 2. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO:

28. In certain embodiments, the extracellular domain comprises a β chain variable region comprising the amino acid sequence set forth in SEQ ID NO: 28. In certain embodiments, the extracellular domain comprises an α chain variable region comprising an amino acid sequence that is at least about 80% (e.g., at least about 85%, at least about 90%, or at least about 95%) homologous to SEQ ID NO: 27, and a β chain variable region comprising an amino acid sequence that is at least about 80% (e.g., at least about 85%, at least about 90%, or at least about 95%) homologous or identical to the amino acid sequence set forth in SEQ ID NO: 28. In certain embodiments, the extracellular domain comprises an α chain variable region comprising the amino acid sequence set forth in SEQ ID NO: 27 and a β chain variable region comprising the amino acid sequence set forth in SEQ ID NO: 28. In certain embodiments, the extracellular domain comprises an α chain variable region CDR1 comprising the amino acid sequence set forth in SEQ ID NO: 21 or a conservative modification thereof, an α chain variable region CDR2 comprising the amino acid sequence set forth in SEQ ID NO: 22 or a conservative modification thereof, and an α chain variable region CDR3 comprising the amino acid sequence set forth in SEQ ID NO: 23 or a conservative modification thereof, as shown in Table 3. In certain embodiments, the extracellular domain comprises an α chain variable region CDR1 comprising the amino acid sequence set forth in SEQ ID NO: 21, an α chain variable region CDR2 comprising the amino acid sequence set forth in SEQ ID NO: 22, and an α chain variable region CDR3 comprising the amino acid sequence set forth in SEQ ID NO: 23. In certain embodiments, the extracellular domain comprises a β chain variable region CDR1 comprising the amino acid sequence set forth in SEQ ID NO: 24 or a conservative modification thereof, a β chain variable region CDR2 comprising the amino acid sequence set forth in SEQ ID NO: 25 or a conservative modification thereof, and a β chain variable region CDR3 comprising the amino acid sequence set forth in SEQ ID NO: 26 or a conservative modification thereof, as shown in Table 3. In certain embodiments, the extracellular domain comprises a β chain variable region CDR1 comprising the amino acid sequence set forth in SEQ ID NO: 24, a β chain variable region CDR2 comprising the amino acid sequence set forth in SEQ ID NO: 25, and a β chain variable region CDR3 comprising the amino acid sequence set forth in SEQ ID NO: 26. In certain embodiments, the extracellular domain comprises an α chain variable region CDR1 comprising the amino acid sequence set forth in SEQ ID NO: 21 or a conservative modification thereof, an α chain variable region CDR2 comprising the amino acid sequence set forth in SEQ ID NO: 22 or a conservative modification thereof, an α chain variable region CDR3 comprising the amino acid sequence set forth in SEQ ID NO: 23 or a conservative modification thereof, a β chain variable region CDR1 comprising the amino acid sequence set forth in SEQ ID NO: 24 or a conservative modification thereof, a β chain variable region CDR2 comprising the amino acid sequence set forth in SEQ ID NO: 25 or a conservative modification thereof, and a β chain variable region CDR3 comprising the amino acid sequence set forth in SEQ ID NO: 26 or a conservative modification thereof. In certain embodiments, the extracellular domain comprises an α chain variable region CDR1 comprising the amino acid sequence set forth in SEQ ID NO: 21, an α chain variable region CDR2 comprising the amino acid sequence set forth in SEQ ID NO: 22, an α chain variable region CDR3 comprising the amino acid sequence set forth in SEQ ID NO: 23, a β chain variable region CDR1 comprising the amino acid sequence set forth in SEQ ID NO: 24, a β chain variable region CDR2 comprising the amino acid sequence set forth in SEQ ID NO: 25, and a β chain variable region CDR3 comprising the amino acid sequence set forth in SEQ ID NO: 26.

TABLE 3
RC8-3

| Antigen | PIK3CA H1047R/L | | |
|---|---|---|---|
| CDRs | 1 | 2 | 3 |
| α-chain | TSDQSYG | QGSYDEQN | CAMREVLDNTDKLIF |
| | [SEQ ID NO: 21] | [SEQ ID NO: 22] | [SEQ ID NO: 23] |
| β-chain | KGHSH | LQKENI | CASSPPEAGLDTEAFF |
| | [SEQ ID NO: 24] | [SEQ ID NO: 25] | [SEQ ID NO: 26] |

| | |
|---|---|
| α-chain | MSLSSLLKVVTASLWLGPGIAQKITQTQPGMFVQEKEAVTLDCTYDT |
| variable | SDQSYGLFWYKQPSSGEMIFLIYQGSYDEQNATEGRYSLNFQKARKS |
| | ANLVISASQLGDSAMYFCAMREVLDNTDKLIFGTGTRLQVFP |
| | [SEQ ID NO: 27] |
| Full α- | MSLSSLLKVVTASLWLGPGIAQKITQTQPGMFVQEKEAVTLDCTYDT |
| chain | SDQSYGLFWYKQPSSGEMIFLIYQGSYDEQNATEGRYSLNFQKARKS |
| | ANLVISASQLGDSAMYFCAMREVLDNTDKLIFGTGTRLQVFPNIQNPE |
| | PAVYQLKDPRSQDSTLCLFTDFDSQINVPKTMESGTFITDKCVLDMK |
| | AMDSKSNGAIAWSNQTSFTCQDIFKETNATYPSSDVPCDATLTEKSFE |
| | TDMNLNFQNLLVIVLRILLLKVAGFNLLMTLRLWSS [SEQ ID NO: 29] |
| β-chain | MDTRVLCCAVICLLGAGLSNAGVMQNPRHLVRRRGQEARLRCSPMK |
| variable | GHSHVYWYRQLPEEGLKFMVYLQKENIIDESGMPKERFSAEFPKEGP |
| | SILRIQQVVRGDSAAYFCASSPPEAGLDTEAFFGQGTRLTVV [SEQ ID |
| | NO: 28] |
| Full β- | MDTRVLCCAVICLLGAGLSNAGVMQNPRHLVRRRGQEARLRCSPMK |
| chain | GHSHVYWYRQLPEEGLKFMVYLQKENIIDESGMPKERFSAEFPKEGP |
| | SILRIQQVVRGDSAAYFCASSPPEAGLDTEAFFGQGTRLTVVEDLRNV |
| | TPPKVSLFEPSKAEIANKQKATLVCLARGFFPDHVELSWWVNGKEVH |
| | SGVCTDPQAYKESNYSYCLSSRLRVSATFWHNPRNHFRCQVQFHGLS |
| | EEDKWPEGSPKPVTQNISAEAWGRADCGITSASYQQGVLSATILYEIL |
| | LGKATLYAVLVSTLVVMAMVKRKNS [SEQ ID NO: 30] |

In certain embodiments, the TCR is a human TCR and specifically binds to a mutant PIK3CA peptide polypeptide (e.g., a human mutant PIK3CA peptide polypeptide), which is designated as LB11-1.

In certain embodiments, the TCR is a human TCR, which comprises an α chain comprising the amino acid sequence set forth in SEQ ID NO: 39 and a β chain comprising the amino acid sequence set forth in SEQ ID NO: 40. SEQ ID NOS: 39 and 40 are provided in Table 4.

In certain embodiments, the extracellular domain of the TCR comprises an α chain variable region and a β chain variable region or CDRs selected from Table 4. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 37, as shown in Table 2. For example, the extracellular domain comprises an α chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 37. In certain embodiments, the extracellular domain comprises an α chain variable region comprising the amino acid sequence set forth in SEQ ID NO: 37. In certain embodiments, the extracellular domain comprises a β chain variable region comprising an amino acid sequence that is at least about 80% (e.g., at least about 85%, at least about 90%, or at least about 95%) homologous or identical to the amino acid sequence set forth in SEQ ID NO: 38, as shown in Table 4. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 38. In certain embodiments, the extracellular domain comprises a β chain variable region comprising the amino acid sequence set forth in SEQ ID NO: 38. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 37, and a β chain variable region comprising an amino acid sequence that is at least about 80% (e.g., at least about 85%, at least about 90%, or at least about 95%) homologous or identical to the amino acid sequence set forth in SEQ ID NO: 38. In certain embodiments, the extracellular domain comprises an a chain variable region comprising the amino acid sequence set forth in SEQ ID NO: 37 and a β chain variable region comprising the amino acid sequence set forth in SEQ ID NO: 38. In certain embodiments, the extracellular domain comprises an α chain variable region CDR1 comprising the amino acid sequence set forth in SEQ ID NO: 31 or a conservative modification thereof, an a chain variable region CDR2 comprising the amino acid sequence set forth in SEQ ID NO: 32 or a conservative modification thereof, and an α chain variable region CDR3 comprising the amino acid sequence set forth in SEQ ID NO: 33 or a conservative modification thereof, as shown in Table 4. In certain embodiments, the extracellular domain comprises an α chain variable region CDR1 comprising the amino acid sequence set forth in SEQ ID NO: 31, an α chain variable region CDR2 comprising the amino acid sequence set forth in SEQ ID NO: 32, and an α chain variable region CDR3 comprising the amino acid sequence set forth in SEQ ID NO: 33. In certain embodiments, the extracellular domain comprises a β chain variable region CDR1 comprising the amino acid sequence set forth in SEQ ID NO: 34 or a conservative modification thereof, a β chain variable region CDR2 comprising the amino acid sequence set forth in SEQ ID NO: 35 or a conservative modification thereof, and a β chain variable region CDR3 comprising the amino acid sequence set forth in SEQ ID NO: 36 or a conservative modification thereof, as shown in Table 4. In certain embodiments, the extracellular domain comprises a β chain variable region CDR1 comprising the amino acid sequence set forth in SEQ ID NO: 34, a β chain variable region CDR2 comprising the amino acid sequence set forth in SEQ ID NO: 35, and a β chain variable region CDR3 comprising the amino acid sequence set forth in SEQ ID NO: 36. In certain embodiments, the extracellular domain comprises an α chain variable region CDR1 comprising the amino acid sequence set forth in SEQ ID NO: 31 or a conservative modification thereof, an α chain variable region CDR2 comprising the amino acid sequence set forth in SEQ ID NO: 32 or a conservative modification thereof, an α chain variable region CDR3 comprising the amino acid sequence set forth in SEQ ID NO: 33 or a conservative modification thereof, a β chain variable region CDR1 comprising the amino acid sequence set forth in SEQ ID NO: 34 or a conservative modification thereof, a β chain variable region CDR2 comprising the amino acid sequence set forth in SEQ ID NO: 35 or a conservative modification thereof, and a β chain variable region CDR3 comprising the amino acid sequence set forth in SEQ ID NO: 36 or a conservative modification thereof. In certain embodiments, the extracellular domain comprises an α chain variable region CDR1 comprising the amino acid sequence set forth in SEQ ID NO: 31, an α chain variable region CDR2 comprising the amino acid sequence set forth in SEQ ID NO: 32, an α chain variable region CDR3 comprising the amino acid sequence set forth in SEQ ID NO: 33, a β chain variable region CDR1 comprising the amino acid sequence set forth in SEQ ID NO: 34, a β chain variable region CDR2 comprising the amino acid sequence set forth in SEQ ID NO: 35, and a β chain variable region CDR3 comprising the amino acid sequence set forth in SEQ ID NO: 36.

TABLE 4
LB11-1

| Antigen | PIK3CA H1047L | | |
|---|---|---|---|
| CDRs | 1 | 2 | 3 |
| α-chain | NSAFQY [SEQ ID NO: | TYSSGN [SEQ ID | CAMNSGGYQKVTF |
| | 31] | NO: 32] | [SEQ ID NO: 33] |
| β-chain | DFQATT [SEQ ID NO: | SNEGSKA [SEQ ID | CSAREQGPLEEQYF |
| | 34] | NO: 35] | [SEQ ID NO: 36] |

| | |
|---|---|
| α-chain | MMKSLRVLLVILWLQLSWVWSQQKEVEQDPGPLSVPEGAIVSLNCT |
| variable | YSNSAFQYFMWYRQYSRKGPELLMYTYSSGNKEDGRFTAQVDKSSK |
| | YISLFIRDSQPSDSATYLCAMNSGGYQKVTFGIGTKLQVIP [SEQ ID |
| | NO: 37] |
| Full α- | MMKSLRVLLVILWLQLSWVWSQQKEVEQDPGPLSVPEGAIVSLNCT |
| chain | YSNSAFQYFMWYRQYSRKGPELLMYTYSSGNKEDGRFTAQVDKSSK |
| | YISLFIRDSQPSDSATYLCAMNSGGYQKVTFGIGTKLQVIPNIQNPEPA |
| | VYQLKDPRSQDSTLCLFTDFDSQINVPKTMESGTFITDKCVLDMKAM |
| | DSKSNGAIAWSNQTSFTCQDIFKETNATYPSSDVPCDATLTEKSFE |
| | TDMNLNFQNLLVIVLRILLLKVAGFNLLMTLRLWSS [SEQ ID NO: 39] |
| β-chain | MLLLLLLLGPAGSGLGAVVSQHPSRVICKSGTSVKIECRSLDFQATTM |
| variable | FWYRQFPKQSLMLMATSNEGSKATYEQGVEKDKFLINHASLTLSTLT |
| | VTSAHPEDSSFYICSACSAREQGPLEEQYFGPGTRLTVT [SEQ ID NO: |
| | 38] |
| Full β- | MLLLLLLLGPAGSGLGAVVSQHPSRVICKSGTSVKIECRSLDFQATTM |
| chain | FWYRQFPKQSLMLMATSNEGSKATYEQGVEKDKFLINHASLTLSTLT |
| | VTSAHPEDSSFYICSACSAREQGPLEEQYFGPGTRLTVTEDLRNVTPP |
| | KVSLFEPSKAEIANKQKATLVCLARGFFPDHVELSWWVNGKEVHSG |
| | VCTDPQAYKESNYSYCLSSRLRVSATFWHNPRNHFRCQVQFHGLSEE |
| | DKWPEGSPKPVTQNISAEAWGRADCGITSASYQQGVLSATILYEILLG |
| | KATLYAVLVSTLVVMAMVKRKNS [SEQ ID NO: 40] |

In certain embodiments, the TCR is a human TCR and specifically binds to a mutant PIK3CA peptide polypeptide (e.g., a human mutant PIK3CA peptide polypeptide), which is designated as LB11-2.

In certain embodiments, the TCR is a human TCR, which comprises an α chain comprising the amino acid sequence set forth in SEQ ID NO: 49 and a β chain comprising the amino acid sequence set forth in SEQ ID NO: 50. SEQ ID NOS: 49 and 50 are provided in Table 5.

In certain embodiments, the extracellular domain of the TCR comprises an α chain variable region and a β chain variable region or CDRs selected from Table 5. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 47, as shown in Table 5. For example, the extracellular domain comprises an α chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 47. In certain embodiments the extracellular domain comprises an α chain variable region comprising the amino acid sequence set forth in SEQ ID NO: 47. In certain embodiments, the extracellular domain comprises a β chain variable region comprising an amino acid sequence that is at least about 80% (e.g., at least about 85%, at least about 90%, or at least about 95%) homologous or identical to the amino acid sequence set forth in SEQ ID NO: 48, as shown in Table 5. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO:

48. In certain embodiments, the extracellular domain comprises a β chain variable region comprising the amino acid sequence set forth in SEQ ID NO: 48. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 47, and a β chain variable region comprising an amino acid sequence that is at least about 80% (e.g., at least about 85%, at least about 90%, or at least about 95%) homologous or identical to the amino acid sequence set forth in SEQ ID NO: 48. In certain embodiments, the extracellular domain comprises an a chain variable region comprising the amino acid sequence set forth in SEQ ID NO: 47 and a β chain variable region comprising the amino acid sequence set forth in SEQ ID NO: 48. In certain embodiments, the extracellular domain comprises an α chain variable region CDR1 comprising the amino acid sequence set forth in SEQ ID NO: 41 or a conservative modification thereof, an a chain variable region CDR2 comprising the amino acid sequence set forth in SEQ ID NO: 42 or a conservative modification thereof, and an α chain variable region CDR3 comprising the amino acid sequence set forth in SEQ ID NO: 43 or a conservative modification thereof, as shown in Table 5. In certain embodiments, the extracellular domain comprises an α chain variable region CDR1 comprising the amino acid sequence set forth in SEQ ID NO: 41, an α chain variable region CDR2 comprising the amino acid sequence set forth in SEQ ID NO: 42, and an α chain variable region CDR3 comprising the amino acid sequence set forth in SEQ ID NO: 43. In certain embodiments, the extracellular domain comprises a β chain variable region CDR1 comprising the amino acid sequence set forth in SEQ ID NO: 44 or a conservative modification thereof, a β chain variable region CDR2 comprising the amino acid sequence set forth in SEQ ID NO: 45 or a conservative modification thereof, and a β chain variable region CDR3 comprising the amino acid sequence set forth in SEQ ID NO: 46 or a conservative modification thereof, as shown in Table 5. In certain embodiments, the extracellular domain comprises a β chain variable region CDR1 comprising the amino acid sequence set forth in SEQ ID NO: 44, a β chain variable region CDR2 comprising the amino acid sequence set forth in SEQ ID NO: 45, and a β chain variable region CDR3 comprising the amino acid sequence set forth in SEQ ID NO: 46. In certain embodiments, the extracellular domain comprises an α chain variable region CDR1 comprising the amino acid sequence set forth in SEQ ID NO: 41 or a conservative modification thereof, an α chain variable region CDR2 comprising the amino acid sequence set forth in SEQ ID NO: 42 or a conservative modification thereof, an α chain variable region CDR3 comprising the amino acid sequence set forth in SEQ ID NO: 43 or a conservative modification thereof, a β chain variable region CDR1 comprising the amino acid sequence set forth in SEQ ID NO: 44 or a conservative modification thereof, a β chain variable region CDR2 comprising the amino acid sequence set forth in SEQ ID NO: 45 or a conservative modification thereof, and a β chain variable region CDR3 comprising the amino acid sequence set forth in SEQ ID NO: 46 or a conservative modification thereof, as shown in Table 5. In certain embodiments, the extracellular domain comprises an α chain variable region CDR1 comprising the amino acid sequence set forth in SEQ ID NO: 41, an α chain variable region CDR2 comprising the amino acid sequence set forth in SEQ ID NO: 42, an α chain variable region CDR3 comprising the amino acid sequence set forth in SEQ ID NO: 43, a β chain variable region CDR1 comprising the amino acid sequence set forth in SEQ ID NO: 44, a β chain variable region CDR2 comprising the amino acid sequence set forth in SEQ ID NO: 45 or a conservative modification thereof, and a β chain variable region CDR3 comprising the amino acid sequence set forth in SEQ ID NO: 46.

TABLE 5
LB11-2

| Antigen | PIK3CA H1047L | | |
|---|---|---|---|
| CDRs | 1 | 2 | 3 |
| α-chain | TISGNEY [SEQ ID | GLKNN [SEQ ID NO: | CIVRVAGSARQLTF |
| | NO: 41] | 42] | [SEQ ID NO: 43] |
| β-chain | SGHDT [SEQ ID | YYEEEE[SEQ ID NO: | CASSFGTATYEQYF |
| | NO: 44] | 45] | [SEQ ID NO: 46] |

| | |
|---|---|
| α-chain | MRLVARVTVFLTFGTIIDAKTTQPPSMDCAEGRAANLPCNHSTISGNE |
| variable | YVYWYRQIHSQGPQYIIHGLKNNETNEMASLIITEDRKSSTLILPHATLR |
| | DTAVYYCIVRVAGSARQLTFGSGTQLTVLP [SEQ ID NO: 47] |
| Full α- | MRLVARVTVFLTFGTIIDAKTTQPPSMDCAEGRAANLPCNHSTISGNE |
| chain | YVYWYRQIHSQGPQYIIHGLKNNETNEMASLIITEDRKSSTLILPHATLR |
| | DTAVYYCIVRVAGSARQLTFGSGTQLTVLPNIQNPEPAVYQLKDPRSQ |
| | DSTLCLFTDFDSQINVPKTMESGTFITDKCVLDMKAMDSKSNGAIAWS |
| | NQTSFTCQDIFKETNATYPSSDVPCDATLTEKSFETDMNLNFQNLLVIV |
| | LRILLLKVAGFNLLMTLRLWSS |
| | [SEQ ID NO: 49] |
| β-chain | MGPGLLCWALLCLLGAGLVDAGVTQSPTHLIKTRGQQVTLRCSPKSG |
| variable | HDTVSWYQQALGQGPQFIFQYYEEEERQRGNFPDRFSGHQFPNYSSEL |
| | NVNALLLGDSALYLCASSFGTATYEQYFGPGTRLTVT [SEQ ID NO: 48] |
| Full β- | MGPGLLCWALLCLLGAGLVDAGVTQSPTHLIKTRGQQVTLRCSPKSG |
| chain | HDTVSWYQQALGQGPQFIFQYYEEEERQRGNFPDRFSGHQFPNYSSEL |
| | NVNALLLGDSALYLCASSFGTATYEQYFGPGTRLTVTEDLRNVTPPKV |
| | SLFEPSKAEIANKQKATLVCLARGFFPDHVELSWWVNGKEVHSGVCT |
| | DPQAYKESNYSYCLSSRLRVSATFWHNPRNHFRCQVQFHGLSEEDKW |
| | PEGSPKPVTQNISAEAWGRADCGITSASYQQGVLSATILYEILLGKATL |
| | YAVLVSTLVVMAMVKRKNS [SEQ ID NO: 50] |

In certain embodiments, the TCR is a human TCR and specifically binds to a mutant PIK3CA peptide polypeptide (e.g., a human mutant PIK3CA peptide polypeptide), which is designated as LB11-3.

In certain embodiments, the TCR is a human TCR, which comprises an α chain comprising the amino acid sequence set forth in SEQ ID NO: 59 and a β chain comprising the amino acid sequence set forth in SEQ ID NO: 60. SEQ ID NOS: 59 and 60 are provided in Table 6.

10 In certain embodiments, the extracellular domain of the TCR comprises an α chain variable region and a β chain variable region or CDRs selected from Table 6. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 57, as shown in Table 6. For example, the extracellular domain comprises an α chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 57. In certain embodiments, the extracellular domain comprises an α chain variable region comprising the amino acid sequence set forth in SEQ ID NO: 57. In certain embodiments, the extracellular domain comprises a β chain variable region comprising an amino acid sequence that is at least about 80% (e.g., at least about 85%, at least about 90%, or at least about 95%) homologous or identical to the amino acid sequence set forth in SEQ ID NO: 58, as shown in Table 6. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 58. In certain embodiments, the extracellular domain comprises a β chain variable region comprising the amino acid sequence set forth in SEQ ID NO: 58. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 57, and a β chain variable region comprising an amino acid sequence that is at least about 80% (e.g., at least about 85%, at least about 90%, or at least about 95%) homologous to SEQ ID NO: 58. In certain embodiments, the extracellular domain comprises an α chain variable region comprising the amino acid sequence set forth in SEQ ID NO: 57 and a β chain variable region comprising the amino acid sequence set forth in SEQ ID NO: 58. In certain embodiments, the extracellular domain comprises an α chain variable region CDR1 comprising the amino acid sequence set forth in SEQ ID NO: 51 or a conservative modification thereof, an α chain variable region CDR2 comprising the amino acid sequence set forth in SEQ ID NO: 52 or a conservative modification thereof, and an α chain variable region CDR3 comprising the amino acid sequence set forth in SEQ ID NO: 53 or a conservative modification thereof, as shown in Table 6. In certain embodiments, the extracellular domain comprises an α chain variable region CDR1 comprising the amino acid sequence set forth in SEQ ID NO: 51, an α chain variable region CDR2 comprising the amino acid sequence set forth in SEQ ID NO: 52, and an α chain variable region CDR3 comprising the amino acid sequence set forth in SEQ ID NO: 53. In certain embodiments, the extracellular domain comprises a β chain variable region CDR1 comprising the amino acid sequence set forth in SEQ ID NO: 54 or a conservative modification thereof, a β chain variable region CDR2 comprising the amino acid sequence set forth in SEQ ID NO: 55 or a conservative modification thereof, and a β chain variable region CDR3 comprising the amino acid sequence set forth in SEQ ID NO: 56 or a conservative modification thereof, as shown in Table 6. In certain embodiments, the extracellular domain comprises a β chain variable region CDR1 comprising the amino acid sequence set forth in SEQ ID NO: 54, a β chain variable region CDR2 comprising the amino acid sequence set forth in SEQ ID NO: 55, and a β chain variable region CDR3 comprising the amino acid sequence set forth in SEQ ID NO: 56. In certain embodiments, the extracellular domain comprises an α chain variable region CDR1 comprising the amino acid sequence set forth in SEQ ID NO: 51 or a conservative modification thereof, an α chain variable region CDR2 comprising the amino acid sequence set forth in SEQ ID NO: 52 or a conservative modification thereof, an α chain variable region CDR3 comprising the amino acid sequence set forth in SEQ ID NO: 53 or a conservative modification thereof, a β chain variable region CDR1 comprising the amino acid sequence set forth in SEQ ID NO: 54 or a conservative modification thereof, a β chain variable region CDR2 comprising the amino acid sequence set forth in SEQ ID NO: 55 or a conservative modification thereof, and a β chain variable region CDR3 comprising the amino acid sequence set forth in SEQ ID NO: 56 or a conservative modification thereof. In certain embodiments, the extracellular domain comprises an α chain variable region CDR1 comprising the amino acid sequence set forth in SEQ ID NO: 51, an α chain variable region CDR2 comprising the amino acid sequence set forth in SEQ ID NO: 52, an α chain variable region CDR3 comprising the amino acid sequence set forth in SEQ ID NO: 53, a β chain variable region CDR1 comprising the amino acid sequence set forth in SEQ ID NO: 54, a β chain variable region CDR2 comprising the amino acid sequence set forth in SEQ ID NO: 55, and a β chain variable region CDR3 comprising the amino acid sequence set forth in SEQ ID NO: 56.

TABLE 6
LB11-3

| Antigen | PIK3CA H1047L | | |
|---|---|---|---|
| CDRs | 1 | 2 | 3 |
| α-chain | DSASNY [SEQ ID | IRSNVGE [SEQ ID | CAASIPGTASKLTF [SEQ |
| | NO: 51] | NO: 52] | ID NO: 53] |
| β-chain | MNHEY [SEQ ID | SMNVEV [SEQ ID | CASSPYRQGSYGYTF |
| | NO: 54] | NO: 55] | [SEQ ID NO: 56] |

| | |
|---|---|
| α-chain | MTSIRAVFIFLWLQLDLVNGENVEQHPSTLSVQEGDSAVIKCTYSDSAS |
| variable | NYFPWYKQELGKGPQLIIDIRSNVGEKKDQRIAVTLNKTAKHFSLHITET |
| | QPEDSAVYFCAASIPGTASKLTFGTGTRLQVTL [SEQ ID NO: 57] |
| Full α- | MTSIRAVFIFLWLQLDLVNGENVEQHPSTLSVQEGDSAVIKCTYSDSAS |
| chain | NYFPWYKQELGKGPQLIIDIRSNVGEKKDQRIAVTLNKTAKHFSLHITET |
| | QPEDSAVYFCAASIPGTASKLTFGTGTRLQVTLNIQNPEPAVYQLKDPR |
| | SQDSTLCLFTDFDSQINVPKTMESGTFITDKCVLDMKAMDSKSNGAIA |
| | WSNQTSFTCQDIFKETNATYPSSDVPCDATLTEKSFETDMNLNFQNLLV |
| | IVLRILLLKVAGFNLL MTLRLWSS [SEQ ID NO: 59] |
| β-chain | MGPQLLGYVVLCLLGAGPLEAQVTQNPRYLITVTGKKLTVTCSQNMN |
| variable | HEYMSWYRQDPGLGLRQIYYSMNVEVTDKGDVPEGYKVSRKEKRNFP |
| | LILESPSPNQTSLYFCASSPYRQGSYGYTFGSGTRLTVV [SEQ ID NO: 58] |
| Full β- | MGPQLLGYVVLCLLGAGPLEAQVTQNPRYLITVTGKKLTVTCSQNMN |
| chain | HEYMSWYRQDPGLGLRQIYYSMNVEVTDKGDVPEGYKVSRKEKRNFP |
| | LILESPSPNQTSLYFCASSPYRQGSYGYTFGSGTRLTVVEDLRNVTPPKV |
| | SLFEPSKAEIANKQKATLVCLARGFFPDHVELSWWVNGKEVHSGVCTD |
| | PQAYKESNYSYCLSSRLRVSATFWHNPRNHFRCQVQFHGLSEEDKWPE |
| | GSPKPVTQNISAEAWGRADCGITSASYQQGVLSATILYEILLGKATLYA |
| | VLVSTLVVMAMVKRKNS [SEQ ID NO: 60] |

In certain embodiments, the TCR is a human TCR and specifically binds to a mutant PIK3CA peptide polypeptide (e.g., a human mutant PIK3CA peptide polypeptide), which is designated as LB11-4.

In certain embodiments, the TCR is a human TCR, which comprises an α chain comprising the amino acid sequence set forth in SEQ ID NO: 69 and a β chain comprising the amino acid sequence set forth in SEQ ID NO: 70. SEQ ID NOS: 69 and 70 are provided in Table 7.

In certain embodiments, the extracellular domain of the TCR comprises an α chain variable region and a β chain variable region or CDRs selected from Table 7. In certain embodiments, the extracellular domain comprises an α chain variable region comprising an amino acid sequence that 10 is at least about 80% (e.g., at least about 85%, at least about 90%, or at least about 95%) homologous or identical to the amino acid sequence set forth in SEQ ID NO: 67, as shown in Table 7. For example, the extracellular domain comprises an α chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 67. In certain embodiments, the extracellular domain comprises an α chain variable region comprising the amino acid sequence set forth in SEQ ID NO: 67. In certain embodiments, the extracellular domain comprises a β chain variable region comprising an amino acid sequence that is at least about 80% (e.g., at least about 85%, at least about 90%, or at least about 95%) homologous or identical to the amino acid sequence set forth in SEQ ID NO: 68, as shown in Table 7. For example, the extracellular domain comprises a β chain variable region comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in the amino acid sequence set forth in SEQ ID NO: 68. In certain embodiments, the extracellular domain comprises a β chain variable region comprising the amino acid sequence set forth in SEQ ID NO: 68. In certain embodiments, the extracellular domain comprises an α chain variable region comprising an amino acid sequence that is at least about 80% (e.g., at least about 85%, at least about 90%, or at least about 95%) homologous or identical to the amino acid sequence set forth in SEQ ID NO: 67, and a β chain variable region comprising an amino acid sequence that is at least about 80% (e.g., at least about 85%, at least about 90%, or at least about 95%) homologous or identical to the amino acid sequence set forth in SEQ ID NO: 68. In certain embodiments, the extracellular domain comprises an α chain variable region comprising the amino acid sequence set forth in SEQ ID NO: 67 and a β chain variable region comprising the amino acid sequence set forth in SEQ ID NO: 68. In certain embodiments, the extracellular domain comprises an α chain variable region CDR1 comprising the amino acid sequence set forth in SEQ ID NO: 61 or a conservative modification thereof, an α chain variable region CDR2 comprising the amino acid sequence set forth in SEQ ID NO: 62 or a conservative modification thereof, and an α chain variable region CDR3 comprising the amino acid sequence set forth in SEQ ID NO: 63 or a conservative modification thereof, as shown in Table 7. In certain embodiments, the extracellular domain comprises an α chain variable region CDR1 comprising the amino acid sequence set forth in SEQ ID NO: 61, an α chain variable region CDR2 comprising the amino acid sequence set forth in SEQ ID NO: 62, and an α chain variable region CDR3 comprising the amino acid sequence set forth in SEQ ID NO: 63. In certain embodiments, the extracellular domain comprises a β chain variable region CDR1 comprising the amino acid sequence set forth in SEQ ID NO: 64 or a conservative modification thereof, a β chain variable region CDR2 comprising the amino acid sequence set forth in SEQ ID NO: 65 or a conservative modification thereof, and a β chain variable region CDR3 comprising the amino acid sequence set forth in SEQ ID NO: 66 or a conservative modification thereof, as shown in Table 7. In certain embodiments, the extracellular domain comprises a β chain variable region CDR1 comprising the amino acid sequence set forth in SEQ ID NO: 64, a β chain variable region CDR2 comprising the amino acid sequence set forth in SEQ ID NO: 65, and a β chain variable region CDR3 comprising the amino acid sequence set forth in SEQ ID NO: 66. In certain embodiments, the extracellular domain comprises an α chain variable region CDR1 comprising the amino acid sequence set forth in SEQ ID NO: 61 or a conservative modification thereof, an α chain variable region CDR2 comprising the amino acid sequence set forth in SEQ ID NO: 62 or a conservative modification thereof, an α chain variable region CDR3 comprising the amino acid sequence set forth in SEQ ID NO: 63 or a conservative modification thereof, a β chain variable region CDR1 comprising the amino acid sequence set forth in SEQ ID NO: 64 or a conservative modification thereof, a β chain variable region CDR2 comprising the amino acid sequence set forth in SEQ ID NO: 65 or a conservative modification thereof, and a β chain variable region CDR3 comprising the amino acid sequence set forth in SEQ ID NO: 66 or a conservative modification thereof. In certain embodiments, the extracellular domain comprises an α chain variable region CDR1 comprising the amino acid sequence set forth in SEQ ID NO: 61, an α chain variable region CDR2 comprising the amino acid sequence set forth in SEQ ID NO: 62, an α chain variable region CDR3 comprising the amino acid sequence set forth in SEQ ID NO: 63, a β chain variable region CDR1 comprising the amino acid sequence set forth in SEQ ID NO: 64, a β chain variable region CDR2 comprising the amino acid sequence set forth in SEQ ID NO: 65, and a β chain variable region CDR3 comprising the amino acid sequence set forth in SEQ ID NO: 66.

TABLE 7
LB11-4

| Antigen | PIK3CA H1047L | | |
|---|---|---|---|
| CDRs | 1 | 2 | 3 |
| α-chain | NTAFDY [SEQ ID | IRPDVSE [SEQ ID | CAASTGNFNKFYF [SEQ |
| | NO: 61] | NO: 62] | ID NO: 63] |
| β-chain | SGHNS [SEQ ID NO: | FNNNVP [SEQ ID | CASNRQGTVTEAFF [SEQ |
| | 64] | NO: 65] | ID NO: 66] |

| | |
|---|---|
| α-chain | MDKILGASFLVLWLQLCWVSGQQKEKSDQQQVKQSPQSLIVQKGGISII |
| variable | NCAYENTAFDYFPWYQQFPGKGPALLIAIRPDVSEKKEGRFTISFNKSA |
| | KQFSLHIMDSQPGDSATYFCAASTGNFNKFYFGSGTKLNVKP [SEQ ID |
| | NO: 67] |
| Full α- | MDKILGASFLVLWLQLCWVSGQQKEKSDQQQVKQSPQSLIVQKGGISII |
| chain | NCAYENTAFDYFPWYQQFPGKGPALLIAIRPDVSEKKEGRFTISFNKSA |
| | KQFSLHIMDSQPGDSATYFCAASTGNFNKFYFGSGTKLNVKPNIQNPEP |
| | AVYQLKDPRSQDSTLCLFTDFDSQINVPKTMESGTFITDKCVLDMKAM |
| | DSKSNGAIAWSNQTSFTCQDIFKETNATYPSSDVPCDATLTEKSFETDM |
| | NLNFQNLLVIVLRILLLKVAGFNLLMTLRLWSS [SEQ ID NO: 69] |
| β-chain | MDSWTFCCVSLCILVAKHTDAGVIQSPRHEVTEMGQEVTLRCKPISGH |
| variable | NSLFWYRQTMMRGLELLIYFNNNVPIDDSGMPEDRFSAKMPNASFSTL |
| | KIQPSEPRDSAVYFCASNRQGTVTEAFFGQGTRLTVV [SEQ ID NO: 68] |
| Full β- | MDSWTFCCVSLCILVAKHTDAGVIQSPRHEVTEMGQEVTLRCKPISGH |
| chain | NSLFWYRQTMMRGLELLIYFNNNVPIDDSGMPEDRFSAKMPNASFSTL |
| | KIQPSEPRDSAVYFCASNRQGTVTEAFFGQGTRLTVVEDLRNVTPPKVS |
| | LFEPSKAEIANKQKATLVCLARGFFPDHVELSWWVNGKEVHSGVCTDP |
| | QAYKESNYSYCLSSRLRVSATFWHNPRNHFRCQVQFHGLSEEDKWPEG |
| | SPKPVTQNISAEAWGRADCGITSASYQQGVLSATILYEILLGKATLYAV |
| | LVSTLVVMAMVKRKNS [SEQ ID NO: 70] |

In certain embodiments, the CDRs regions described above are delineated using the IMGT numbering system (accessible at www.imgt.org/IMGTScientificChart/Numbering/IMGTIGVLsuperfamily.html, www.imgt.org/IMGTindex/numbering.php).

As used herein, the term “a conservative sequence modification” refers to an amino acid modification that does not significantly affect or alter the binding characteristics of the presently disclosed TCR comprising the amino acid sequence. Conservative modifications can include amino acid substitutions, additions and deletions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered TCR can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

In certain embodiments, the α chain variable region and/or the β chain variable region amino acid sequences have at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to the specified sequences (e.g., SEQ ID NOs: 7, 8, 17, 18, 27, 28, 37, 38, 47, 48, 57, 58, 67 and 68) contain substitutions (e.g., conservative substitutions), insertions, or deletions relative to the specified sequence(s), but retain the ability to bind to a mutant PIK3CA peptide (e.g., a human mutant PIK3CA peptide).

In certain embodiments, the extracellular domain specifically binds to a mutant PIK3CA peptide (e.g., a human mutant PIK3CA peptide) and not the corresponding wildtype peptide sequence.

In certain embodiments, a total of 1 to 10 amino acids are substituted, inserted and/or deleted in SEQ ID NOs: 7, 8, 17, 18, 27, 28, 37, 38, 47, 48, 57, 58, 67 or 68. In certain embodiments, substitutions, insertions, or deletions occur in regions outside the CDRs of the extracellular domain. In certain embodiments, the extracellular domain comprises an α chain variable region and/or a β chain variable region sequence selected from the group consisting of SEQ ID NOs: 7, 8, 17, 18, 27, 28, 37, 38, 47, 48, 57, 58, 67 and 68, including post-translational modifications of that sequence (SEQ ID NO: 7, 8, 17, 18, 27, 28, 37, 38, 47, 48, 57, 58, 67 or 68).

As used herein, the percent homology between two amino acid sequences is equivalent to the percent identity between the two sequences. The percent identity between the two sequences is a function of the number of identical positions shared by the sequences (i.e., % homology= # of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

In certain embodiments, the extracellular domain binds to the same epitope on a mutant PIK3CA peptide (e.g., a human mutant PIK3CA peptide) as the reference TCR. For example, the extracellular domain of a presently disclosed TCR binds to the same epitope on a mutant PIK3CA peptide (e.g., a human mutant PIK3CA peptide) as a reference TCR or a functional fragment thereof comprising the an α chain variable region CDR1, CDR2, and CDR3 sequences and the a β chain variable region CDR1, CDR2, and CDR3 sequences of, for example, any one of the presently disclosed TCRs (e.g., RC8-1, RC8-2, RC8-3, LB11-1, LB11-2, LB11-3 and LB11-4). In certain embodiments, the extracellular domain of a presently disclosed TCR binds to the same epitope on a mutant PIK3CA peptide (e.g., a human mutant PIK3CA peptide) as a reference TCR or a functional fragment thereof comprising the an α chain variable region and a β chain variable region sequences of, for example, any one of the presently disclosed TCRs (e.g., RC8-1, RC8-2, RC8-3, LB11-1, LB11-2, LB11-3 and LB11-4).

It is well known in the art that the CDR3 domain, independently from the CDR1 and/or CDR2 domain(s), alone can determine the binding specificity of a TCR or a functional fragment thereof, for a cognate antigen and that multiple TCRs can predictably be generated having the same binding specificity based on a common CDR3 sequence.

In certain embodiments, the extracellular domain comprises an α chain variable region CDR3 comprising the amino acid sequence set forth in SEQ ID NOS: 3, 13, 23, 33, 43, 53, 63 or a conservative modification thereof; and a β chain variable region CDR3 comprising the amino acid sequence set forth in SEQ ID NOS: 6, 16, 26, 36, 46, 56, 66 or a conservative modification thereof.

In certain embodiments, the extracellular domain comprises an α chain variable region CDR3 comprising the amino acid sequence set forth in SEQ ID NO: 3 or a conservative modification thereof; and a β chain variable region CDR3 comprising the amino acid sequence set forth in SEQ ID NO: 6 or a conservative modification thereof.

In certain embodiments, the extracellular domain comprises an α chain variable region CDR3 comprising the amino acid sequence set forth in SEQ ID NO: 13 or a conservative modification thereof; and a β chain variable region CDR3 comprising the amino acid sequence set forth in SEQ ID NO: 16 or a conservative modification thereof.

In certain embodiments, the extracellular domain comprises an α chain variable region CDR3 comprising the amino acid sequence set forth in SEQ ID NO: 23 or a conservative modification thereof; and a β chain variable region CDR3 comprising the amino acid sequence set forth in SEQ ID NO: 26 or a conservative modification thereof.

In certain embodiments, the extracellular domain comprises an α chain variable region CDR3 comprising the amino acid sequence set forth in SEQ ID NO: 33 or a conservative modification thereof; and a β chain variable region CDR3 comprising the amino acid sequence set forth in SEQ ID NO: 36 or a conservative modification thereof.

In certain embodiments, the extracellular domain comprises an α chain variable region CDR3 comprising the amino acid sequence set forth in SEQ ID NO: 43 or a conservative modification thereof; and a β chain variable region CDR3 comprising the amino acid sequence set forth in SEQ ID NO: 46 or a conservative modification thereof.

In certain embodiments, the extracellular domain comprises an α chain variable region CDR3 comprising the amino acid sequence set forth in SEQ ID NO: 53 or a conservative modification thereof; and a β chain variable region CDR3 comprising the amino acid sequence set forth in SEQ ID NO: 56 or a conservative modification thereof.

In certain embodiments, the extracellular domain comprises an α chain variable region CDR3 comprising the amino acid sequence set forth in SEQ ID NO: 63 or a conservative modification thereof; and a β chain variable region CDR3 comprising the amino acid sequence set forth in SEQ ID NO: 66 or a conservative modification thereof.

The extracellular domain can further comprise an α chain variable region CDR2 comprising the amino acid sequence set forth in SEQ ID NOS: 2, 12, 22, 32, 42, 52, 62 or a conservative modification thereof; and a β chain variable region CDR2 comprising the amino acid sequence set forth in SEQ ID NOS: 5, 15, 25, 35, 45, 55, 65 or a conservative modification thereof.

In certain embodiments, the extracellular domain comprises an α chain variable region CDR2 comprising the amino acid sequence set forth in SEQ ID NO: 2 or a conservative modification thereof; and a β chain variable region CDR2 comprising the amino acid sequence set forth in SEQ ID NO: 5 or a conservative modification thereof.

In certain embodiments, the extracellular domain comprises an α chain variable region CDR2 comprising the amino acid sequence set forth in SEQ ID NO: 12 or a conservative modification thereof; and a β chain variable region CDR2 comprising the amino acid sequence set forth in SEQ ID NO: 15 or a conservative modification thereof.

In certain embodiments, the extracellular domain comprises an α chain variable region CDR2 comprising the amino acid sequence set forth in SEQ ID NO: 22 or a conservative modification thereof; and a β chain variable region CDR2 comprising the amino acid sequence set forth in SEQ ID NO: 25 or a conservative modification thereof.

In certain embodiments, the extracellular domain comprises an α chain variable region CDR2 comprising the amino acid sequence set forth in SEQ ID NO: 32 or a conservative modification thereof; and a β chain variable region CDR2 comprising the amino acid sequence set forth in SEQ ID NO: 35 or a conservative modification thereof.

In certain embodiments, the extracellular domain comprises an α chain variable region CDR2 comprising the amino acid sequence set forth in SEQ ID NO: 42 or a conservative modification thereof; and a β chain variable region CDR2 comprising the amino acid sequence set forth in SEQ ID NO: 45 or a conservative modification thereof.

In certain embodiments, the extracellular domain comprises an α chain variable region CDR2 comprising the amino acid sequence set forth in SEQ ID NO: 52 or a conservative modification thereof; and a β chain variable region CDR2 comprising the amino acid sequence set forth in SEQ ID NO: 55 or a conservative modification thereof.

In certain embodiments, the extracellular domain comprises an α chain variable region CDR2 comprising the amino acid sequence set forth in SEQ ID NO: 62 or a conservative modification thereof; and a β chain variable region CDR2 comprising the amino acid sequence set forth in SEQ ID NO: 65 or a conservative modification thereof.

The extracellular domain can further comprise an α chain variable region CDR1 comprising the amino acid sequence set forth in SEQ ID NOS: 1, 11, 21, 31, 41, 51, 61 or a conservative modification thereof; and a β chain variable region CDR1 comprising the amino acid sequence set forth in SEQ ID NOS: 4, 14, 24, 34, 44, 54, 64 or a conservative modification thereof.

In certain embodiments, the extracellular domain comprises an α chain variable region CDR1 comprising the amino acid sequence set forth in SEQ ID NO: 1 or a conservative modification thereof; and a β chain variable region CDR1 comprising the amino acid sequence set forth in SEQ ID NO: 4 or a conservative modification thereof.

In certain embodiments, the extracellular domain comprises an α chain variable region CDR1 comprising the amino acid sequence set forth in SEQ ID NO: 11 or a conservative modification thereof; and a β chain variable region CDR1 comprising the amino acid sequence set forth in SEQ ID NO: 14 or a conservative modification thereof.

In certain embodiments, the extracellular domain comprises an α chain variable region CDR1 comprising the amino acid sequence set forth in SEQ ID NO: 21 or a conservative modification thereof; and a β chain variable region CDR1 comprising the amino acid sequence set forth in SEQ ID NO: 24 or a conservative modification thereof.

In certain embodiments, the extracellular domain comprises an α chain variable region CDR1 comprising the amino acid sequence set forth in SEQ ID NO: 31 or a conservative modification thereof; and a β chain variable region CDR1 comprising the amino acid sequence set forth in SEQ ID NO: 34 or a conservative modification thereof.

In certain embodiments, the extracellular domain comprises an α chain variable region CDR1 comprising the amino acid sequence set forth in SEQ ID NO: 41 or a conservative modification thereof; and a β chain variable region CDR1 comprising the amino acid sequence set forth in SEQ ID NO: 44 or a conservative modification thereof.

In certain embodiments, the extracellular domain comprises an α chain variable region CDR1 comprising the amino acid sequence set forth in SEQ ID NO: 51 or a conservative modification thereof; and a β chain variable region CDR1 comprising the amino acid sequence set forth in SEQ ID NO: 54 or a conservative modification thereof.

In certain embodiments, the extracellular domain comprises an α chain variable region CDR1 comprising the amino acid sequence set forth in SEQ ID NO: 61 or a conservative modification thereof; and a β chain variable region CDR1 comprising the amino acid sequence set forth in SEQ ID NO: 64 or a conservative modification thereof.

In some embodiments, the TCR of the presently disclosed subject matter can further comprise an inducible promoter, for expressing nucleic acid sequences in human cells. Promoters for use in expressing TCR genes can be a constitutive promoter, such as ubiquitin C (UbiC) promoter.

The presently disclosed subject matter also provides an isolated nucleic acid molecule encoding the mutant PIK3CA-targeted TCR described herein or a functional portion thereof. In certain embodiments, the isolated nucleic acid molecule encodes both an α chain and a β chain of a TCR. In certain embodiments, the α chain and the β chain are separated by a self-cleavage peptide, e.g., a 2A-peptide. In certain embodiments, the α chain and the β chain are separated by a furin-2A-peptide, e.g., a peptide set forth in SEQ ID NO: 84.

[SEQ ID NO: 84]
RAKRSGSGATNFSLLKQAGDVEENPGP

In certain embodiments, the isolated nucleic acid molecule encodes a functional portion/fragment of a presently disclosed mutant PIK3CA-targeted TCR. As used herein, the term “functional portion” or “functional fragment” refers to any portion, part or fragment of a presently disclosed mutant PIK3CA-targeted TCR, which portion, part or fragment retains the biological activity of the mutant PIK3CA-targeted TCR (the parent TCR). For example, functional portions encompass the portions, parts or fragments of a presently disclosed mutant PIK3CA-targeted TCR that retains the ability to recognize a target cell, to treat a disease, e.g., breast cancer, to a similar, same, or even a higher extent as the parent TCR. In certain embodiments, an isolated nucleic acid molecule encoding a functional portion of a presently disclosed mutant PIK3CA-targeted TCR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent TCR.

TCRs with Modifications within CDRs

In certain embodiments, a presently disclosed TCR (or a functional fragment thereof) comprises an α chain variable region comprising CDR1, CDR2 and CDR3 sequences and a β chain variable region comprising CDR1, CDR2 and CDR3 sequences, wherein one or more of these CDR sequences comprise specified amino acid sequences based on the TCRs (or a functional fragments thereof) described herein (see Tables 1-7), or modifications thereof, and wherein the TCRs (or a functional fragments thereof) retain the desired functional properties of the mutant PIK3CA peptide-specific TCRs (or a functional fragments thereof) of the presently disclosed subject matter.

In certain embodiments, a presently disclosed TCR (or a functional fragment thereof) comprises an α chain constant region and a β chain constant region, wherein at least one of the constant regions comprises specified amino acid sequences based on the TCRs (or a functional fragments thereof) described herein (see Tables 1-7), or modifications thereof, and wherein the TCR (or a functional fragment thereof) retains the desired functional properties of the mutant PIK3CA peptide-specific TCRs (or a functional fragments thereof) of the presently disclosed subject matter.

In certain embodiments, such modifications do not significantly affect or alter the binding characteristics of the TCR containing the amino acid sequence. Non-limiting examples of such modifications include amino acid substitutions, additions and deletions. Modifications can be introduced into the presently disclosed TCR or a functional fragment thereof by standard techniques known in the art, such as site-directed mutagenesis and PCR-mediated mutagenesis.

The modifications can be conservative modifications, non-conservative modifications, or mixtures of conservative and non-conservative modifications. As discussed above, conservative amino acid substitutions are ones in which the amino acid residue is replaced with an amino acid residue having a similar side chain. Families of amino acid residues having similar side chains have been defined in the art. Exemplary conservative amino acid substitutions are shown in Table 8. In certain embodiments, amino acid substitutions may be introduced into a TCR of interest and the products screened for a desired activity, e.g., retained/improved antigen binding, decreased immunogenicity, or improved ADCC or CDC.

TABLE 8

| Original | Exemplary conservative |
|---|---|
| Residue | amino acid Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

In certain embodiments, one or more amino acid residues within a CDR region can be replaced with other amino acid residues from the same group and the altered TCR can be tested for retained function using the functional assays described herein.

Non-conservative substitutions entail exchanging a member of one of these classes for another class.

In certain embodiments, no more than one, no more than two, no more than three, no more than four, no more than five residues within a specified sequence or a CDR region are altered.

In certain embodiments, one or more amino acid residues within a constant region of a TCR can be modified to enhance stability and/or cell surface expression of the TCR. In certain embodiments, no more than one, no more than two, no more than three, no more than four, no more than five residues within a specified sequence or a constant region are altered. In certain embodiments, the modification includes but is not limited to, murinization, cysteine modification and transmembrane modification (see Cohen et al. Enhanced antitumor activity of murine-human hybrid T-cell receptor (TCR) in human lymphocytes is associated with improved pairing and TCR/CD3 stability, Cancer Res. 2006;66 (17): 8878-8886; Cohen et al. Enhanced antitumor activity of T cells engineered to express T-cell receptors with a second disulfide bond, Cancer Res. 2007; 67 (8): 3898-3903; Kuball et al. Facilitating matched pairing and expression of TCR chains introduced into human T cells, Blood 2007; 109 (6): 2331-2338; Haga-Friedman et al. Incorporation of transmembrane hydrophobic mutations in the TCR enhance its surface expression and T cell functional avidity, Journal of immunology 2012; 188 (11): 5538-5546, the contents of each of which are incorporated by reference in their entireties).

In certain embodiments, a TCR disclosed herein comprises a modified TCR α-chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 80 or 81. In certain embodiments, the modified TCR α-chain constant region comprises an amino acid sequence set forth in SEQ ID NO: 80. In certain embodiments, the modified TCR α-chain constant region comprises an amino acid sequence set forth in SEQ ID NO: 81.

In certain embodiments, a TCR disclosed herein comprises a modified TCR β-chain constant region that comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82 or 83. In certain embodiments, the modified TCR β-chain constant region comprises an amino acid sequence set forth in SEQ ID NO: 82. In certain embodiments, the modified TCR β-chain constant region comprises an amino acid sequence set forth in SEQ ID NO: 83.

Human α-chain constant region:
[SEQ ID NO: 80]
NIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEK
SFETDTNLNFQNLSVIGFRILLLKVAGENLLMTLRLWSS
Mouse α-chain constant region:
(cysteine-modification and LVL modification in
[SEQ ID NO: 81]
NIQNPEPAVYQLKDPRSQDSTLCLFTDFDSQINVPKTMESGTFITDKCVLD
MKAMDSKSNGAIAWSNOTSFTCQDIFKETNATYPSSDVPCDATLTEKSFET
DMNLNFQNLLVIVLRILLLKVAGENLLMTLRLWSS
Human β-chain constant region:
[SEQ ID NO: 82]
EDLNKVFPPEVAVFEPSEAEISHTQKATLVCLATGFFPDHVELSWWVNGKE
VHSGVSTDPQPLKEQPALNDSRYCLSSRLRVSATFWQNPRNHFRCQVQFYG
LSENDEWTQDRAKPVTQIVSAEAWGRADCGFTSVSYQQGVLSATILYEILL
GKATLYAVLVSALVLMAMVKRKDF
Mouse β-chain constant region:
(cysteine-modification underlined)
[SEQ ID NO: 83]
EDLRNVTPPKVSLFEPSKAEIANKQKATLVCLARGFFPDHVELSWWVNGKE
VHSGVCTDPQAYKESNYSYCLSSRLRVSATFWHNPRNHFRCQVQFHGLSEE
DKWPEGSPKPVTQNISAEAWGRADCGITSASYQQGVLSATILYEILLGKAT
LYAVLVSTLVVMAMVKRKNS

### V. Immunoresponsive Cells

The presently disclosed subject matter provides cells comprising a TCR targeting a mutant PIK3CA peptide. Such cells are administered to a human subject in need thereof for treating and/or preventing a malignancy, e.g., breast cancer

The presently disclosed subject matter provides immunoresponsive cells comprising a TCR that specifically binds to a mutant PIK3CA peptide (e.g., a human mutant PIK3CA peptide) as described above.

The immunoresponsive cells can be transduced with a presently disclosed TCR such that the cells express the TCR. The presently disclosed subject matter also provides methods of using such cells for the treatment of a malignancy, e.g., breast cancer.

The immunoresponsive cells of the presently disclosed subject matter can be cells of the lymphoid lineage. The lymphoid lineage, comprising B, T and natural killer (NK) cells, provides for the production of TCRs, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the TCR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The immunoresponsive cells of the presently disclosed subject matter can express a TCR that specifically binds to a mutant PIK3CA peptide (e.g., a human mutant PIK3CA peptide), for the treatment of cancer, e.g., breast cancer. Such immunoresponsive cells can be administered to a subject (e.g., a human subject) in need thereof for the treatment of cancer, e.g., breast cancer. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

A presently disclosed immunoresponsive cell can further include at least one recombinant or exogenous co-stimulatory ligand. For example, a presently disclosed immunoresponsive cell can be further transduced with at least one co-stimulatory ligand, such that the immunoresponsive cell co-expresses or is induced to co-express the PIK3CA-targeted TCR and the at least one co-stimulatory ligand. The interaction between the PIK3CA-targeted TCR and at least one co-stimulatory ligand provides a non-antigen-specific signal important for full activation of an immunoresponsive cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD30L/CD153, tumor necrosis factor beta (TNFβ)/lymphotoxin-alpha (LTα), lymphotoxin-beta (LTB), CD257/B cell-activating factor (BAFF)/Blys/THANK/Tall-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, but are not limited to, CD80 and CD86, both ligands for CD28, PD-L1/(B7-H1) that ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the immunoresponsive cell comprises one recombinant co-stimulatory ligand that is 4-1BBL. In certain embodiments, the immunoresponsive cell comprises two recombinant co-stimulatory ligands that are 4-1BBL and CD80.

Furthermore, a presently disclosed immunoresponsive cell can further comprise at least one exogenous cytokine. For example, a presently disclosed immunoresponsive cell can be further transduced with at least one cytokine, such that the immunoresponsive cell secretes the at least one cytokine as well as expresses the mutant PIK3CA-targeted TCR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The mutant PIK3CA peptide-specific or mutant PIK3CA-targeted human lymphocytes that can be used in peripheral donor lymphocytes, e.g., those disclosed in Sadelain, M., et al. 2003 Nat Rev Cancer 3:35-45 (disclosing peripheral donor lymphocytes genetically modified to express TCRs), in Morgan, R. A., et al. 2006 Science 314:126-129 (disclosing peripheral donor lymphocytes genetically modified to express a full-length tumor antigen-recognizing T cell receptor complex comprising the a and B heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially.

A large proportion of terminally differentiated cells can be initially removed by a relatively crude separation. For example, magnetic bead separations can be used initially to remove large numbers of irrelevant cells. Preferably, at least about 80%, usually at least about 70% of the total hematopoietic cells will be removed prior to cell isolation.

Procedures for separation include, but are not limited to, density gradient centrifugation; resetting; coupling to particles that modify cell density; magnetic separation with TCR-coated magnetic beads; affinity chromatography; cytotoxic agents joined to or used in conjunction with a mAb, including, but not limited to, complement and cytotoxins; and panning with TCR attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising 2% fetal calf serum (FCS) or 0.2% bovine serum albumin (BSA) or any other suitable, preferably sterile, isotonic medium.

### VI. Vectors

Genetic modification of immunoresponsive cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the mutant PIK3CA-targeted TCR can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide mutant PIK3CA-targeted TCR expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Transducing viral vectors can be used to express a co-stimulatory ligand and/or secrets a cytokine (e.g., 4-1BBL and/or IL-12) in an immunoresponsive cell. Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263 267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

In certain non-limiting embodiments, the vector expressing a presently disclosed mutant PIK3CA-targeted TCR is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

VII. Genomic Integration into Immunoresponsive Cell

In certain embodiments, a TCR can be integrated into a selected locus of the genome of an immunoresponsive cell. Any targeted genome editing methods can be used to integrate the TCR in selected loci of the genome of an immunoresponsive cell. In certain embodiments, the expression of the TCR is driven by an endogenous promoter/enhancer within or near the locus. In certain embodiments, the expression of the TCR is driven by an exogenous promoter integrated into the locus. The locus where the TCR is integrated is selected based on the expression level of the genes within the locus, and timing of the gene expression of the genes within the locus. The expression level and timing can vary under different stages of cell differentiation and mitogen/cytokine microenvironment, which are among the factors to be considered when making the selection.

In certain embodiments, the CRISPR system is used to integrate the TCR in selected loci of the genome of an immunoresponsive cell. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying TCR expression cassette need also be designed for each application, as it must overlap with the sequences on either side of the cut and code for the insertion sequence. Multiple crRNA's and the tracrRNA can be packaged together to form a single-guide

RNA (sgRNA). This sgRNA can be joined together with the Cas9 gene and made into a plasmid in order to be transfected into cells. Methods of using the CRISPR system are described, for example, in WO 2014093661 A2, WO 2015123339 A1 and WO 2015089354 A1, which are incorporated by reference in their entireties.

In certain embodiments, zinc-finger nucleases are used to integrate the TCR in selected loci of the genome of an immunoresponsive cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying TCR expression cassette, ZFNs can be used to insert the TCR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome. Methods of using the ZFN system are described, for example, in WO 2009146179 A1, WO 2008060510 A2 and CN 102174576 A, which are incorporated by reference in their entireties.

In certain embodiments, the TALEN system is used to integrate the TCR in selected loci of the genome of an immunoresponsive cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. Methods of using the TALEN system are described, for example, in WO 2014134412 A1, WO 2013163628 A2 and WO 2014040370 A1, which are incorporated by reference in their entireties.

Methods for delivering the genome editing agents can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within the selected locus, or anywhere that can influence gene expression of the integrated TCR. In certain embodiments, the modification is introduced upstream of the transcriptional start site of the integrated TCR. In certain embodiments, the modification is introduced between the transcriptional start site and the protein coding region of the integrated TCR) In certain embodiments, the modification is introduced downstream of the protein coding region of the integrated TCR.

### VIII. Polypeptides and Analogs and Polynucleotides

Also included in the presently disclosed subject matter are TCRs that specifically binds to a mutant PIK3CA peptide (e.g., a human mutant PIK3CA peptide) functional fragments thereof, and polynucleotides encoding thereof that are modified in ways that enhance their anti-tumor activity when expressed in an immunoresponsive cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the invention. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged therapeutic effect upon administration. Assays for measuring functional activity include, but are not limited to, those described in the Examples below.

In accordance with the presently disclosed subject matter, the polynucleotides encoding an extracellular domain that specifically binds to a mutant PIK3CA peptide (e.g., a human mutant PIK3CA peptide) can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### X. Administration

The presently disclosed PIK3CA-targeted TCRs and immunoresponsive cells comprising thereof can be provided systemically or directly to a subject for treating or preventing a neoplasm. In certain embodiments, mutant PIK3CA-targeted TCRs and immunoresponsive cells comprising thereof are directly injected into an organ of interest (e.g., an organ affected by a neoplasm). Alternatively or additionally, the mutant PIK3CA-targeted TCRs and immunoresponsive cells comprising thereof are provided indirectly to the organ of interest, for example, by administration into the circulatory system (e.g., the tumor vasculature). Expansion and differentiation agents can be provided prior to, during or after administration of cells and compositions to increase production of T cells in vitro or in vivo.

Mutant PIK3CA-targeted TCRs and immunoresponsive cells comprising thereof of the presently disclosed subject matter can be administered in any physiologically acceptable vehicle, normally intravascularly, although they may also be introduced into bone or other convenient site where the cells may find an appropriate site for regeneration and differentiation (e.g., thymus). In certain embodiments, at least about 1×10⁵ cells can be administered, eventually reaching about 1×10¹⁰ or more. In certain embodiments, at least about 1×10⁶ cells can be administered. A cell population comprising immunoresponsive cells comprising an mutant PIK3CA-targeted TCR can comprise a purified population of cells. Those skilled in the art can readily determine the percentage of immunoresponsive cells in a cell population using various well-known methods, such as fluorescence activated cell sorting (FACS). The ranges of purity in cell populations comprising genetically modified immunoresponsive cells comprising a mutant PIK3CA peptide-specific TCR can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The immunoresponsive cells can be introduced by injection, catheter, or the like. If desired, factors can also be included, including, but not limited to, interleukins, e.g. IL-2, IL-3, IL 6, IL-11, IL-7, IL-12, IL-15, IL-21, as well as the other interleukins, the colony stimulating factors, such as G-, M-and GM-CSF, interferons, e.g., γ-interferon.

In certain embodiments, compositions of the presently disclosed subject matter comprise pharmaceutical compositions comprising immunoresponsive cells comprising a mutant PIK3CA-targeted TCR and a pharmaceutically acceptable carrier. Administration can be autologous or non-autologous. For example, immunoresponsive cells comprising a mutant PIK3CA-targeted TCR and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising immunoresponsive cells comprising a mutant PIK3CA-targeted TCR), it can be formulated in a unit dosage injectable form (solution, suspension, emulsion).

In certain embodiments, compositions of the presently disclosed subject matter can further comprise a pharmaceutically acceptable carrier.

### XI. Formulations

Immunoresponsive cells comprising a presently disclosed mutant PIK3CA-targeted TCR and compositions comprising thereof of the presently disclosed subject matter can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising immunoresponsive cells comprising a presently disclosed mutant PIK3CA-targeted TCR, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the immunoresponsive cells comprising a generally mutant PIK3CA-targeted TCR of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the immunoresponsive cells as describe in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the immunoresponsive cells of the presently disclosed subject matter is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ immunoresponsive cells of the presently disclosed subject matter are administered to a subject. More effective cells may be administered in even smaller numbers. In some embodiments, at least about 1×10⁸, about 2×10⁸, about 3×10⁸, about 4×10⁸, and about 5×10⁸ immunoresponsive cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### XII. Methods of Treatment

Provided herein are methods for treating a malignancy in a subject. The methods comprise administering the presently disclosed cells in an amount effective to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

For adoptive immunotherapy using antigen-specific T cells, cell doses in the range of about 10⁶ to about 10¹⁰ (e.g., about 10⁹ or about 10⁶) are typically infused. Upon administration of the immunoresponsive cells into the subject and subsequent differentiation, the immunoresponsive cells are induced that are specifically directed against one specific antigen (e.g., mutant PIK3CA peptides). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and the compositions comprising thereof are intravenously administered to the subject in need.

The presently disclosed subject matter provides various methods of using the immunoresponsive cells (e.g., T cells) comprising a mutant PIK3CA-targeted TCR. For example, the presently disclosed subject matter provides methods of reducing tumor burden in a subject. In certain non-limiting examples, the method of reducing tumor burden comprises administering an effective amount of the presently disclosed immunoresponsive cell to the subject, thereby inducing tumor cell death in the subject. The presently disclosed immunoresponsive cell can reduce the number of tumor cells, reduce tumor size, and/or eradicate the tumor in the subject.

The presently disclosed subject matter also provides methods of increasing or lengthening survival of a subject having a neoplasm. In certain non-limiting example, the method of increasing or lengthening survival of a subject having a neoplasm comprises administering an effective amount of the presently disclosed immunoresponsive cell to the subject, thereby increasing or lengthening survival of the subject.

Cancers whose growth may be inhibited using the immunoresponsive cells of the presently disclosed subject matter comprise cancers typically responsive to immunotherapy. Non-limiting examples of cancers for treatment include any PIK3CA mutated cancers, including and are not limited to, breast cancer, endometrial cancer, cervical cancer, anal cancer, bladder cancer, colorectal cancer, head and neck squamous cell carcinoma, nonmelanoma skin cancer and salivary gland cancer. In certain embodiments, the cancer is breast cancer.

Additionally, the presently disclosed subject matter provides methods of increasing immune-activating cytokine production in response to a cancer cell in a subject. In certain embodiments, the method comprises administering the presently disclosed immunoresponsive cell to the subject. The immune-activating cytokine can be granulocyte macrophage colony stimulating factor (GM-CSF), IFN-α, IFN-β, IFN-γ, TNF-α, IL-2, IL-3, IL-6, IL-11, IL-7, IL-12, IL-15, IL-21, interferon regulatory factor 7 (IRF7), and combinations thereof. In certain embodiments, the immunoresponsive cells including a mutant PIK3CA peptide-specific TCR of the presently disclosed subject matter increase the production of GM-CSF, IFN-γ, and/or TNF-α.

Suitable human subjects for therapy typically comprise two treatment groups that can be distinguished by clinical criteria. Subjects with “advanced disease” or “high tumor burden” are those who bear a clinically measurable tumor (e.g., breast cancer). A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor (e.g., breast cancer).

A second group of suitable subjects is known in the art as the “adjuvant group.” These are individuals who have had a history of neoplasm (e.g., breast cancer), but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasm. Features typical of high-risk subgroups are those in which the tumor (e.g., breast cancer) has invaded neighboring tissues, or who show involvement of lymph nodes. Another group has a genetic predisposition to neoplasm (e.g., breast cancer) but has not yet evidenced clinical signs of neoplasm (e.g., breast cancer). For instance, women testing positive for a genetic mutation associated with breast cancer, but still of childbearing age, can wish to receive one or more of the TCRs described herein in treatment prophylactically to prevent the occurrence of neoplasm until it is suitable to perform preventive surgery.

The subjects can have an advanced form of disease (e.g., breast cancer), in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

### XIII. Kits

The presently disclosed subject matter provides kits for the treatment or prevention of a malignancy (e.g., breast cancer). In certain embodiments, the kit comprises a therapeutic or prophylactic composition containing an effective amount of an immunoresponsive cell comprising a mutant PIK3CA-targeted TCR in unit dosage form. In particular embodiments, the cells further expresses at least one co-stimulatory ligand.

If desired, the immunoresponsive cell can be provided together with instructions for administering the cell to a subject having or at risk of developing a malignancy (e.g., breast cancer). The instructions will generally include information about the use of the composition for the treatment or prevention of a malignancy (e.g., breast cancer). In other embodiments, the instructions include at least one of the following: description of the therapeutic agent; dosage schedule and administration for treatment or prevention of a malignancy (e.g., breast cancer) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1: Generation of Mutant PIK3CA-Targeted TCRs

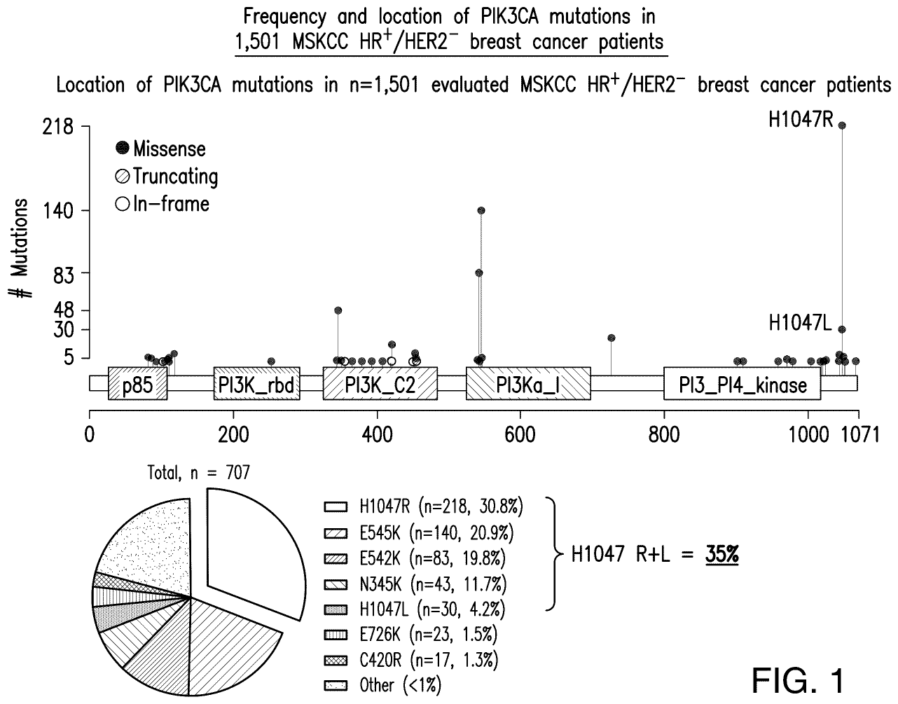

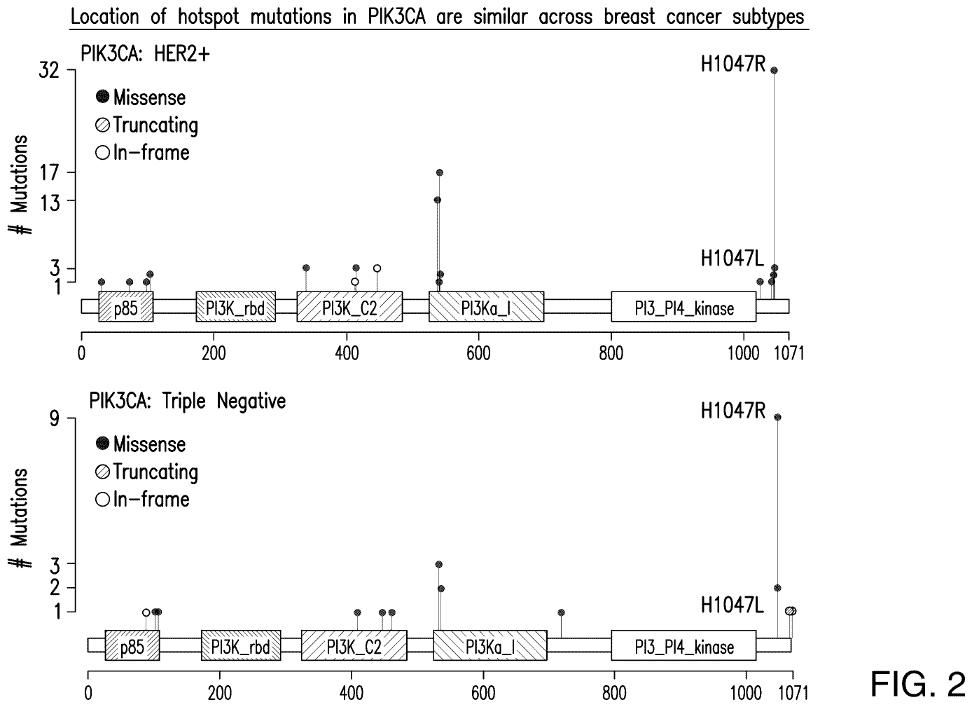

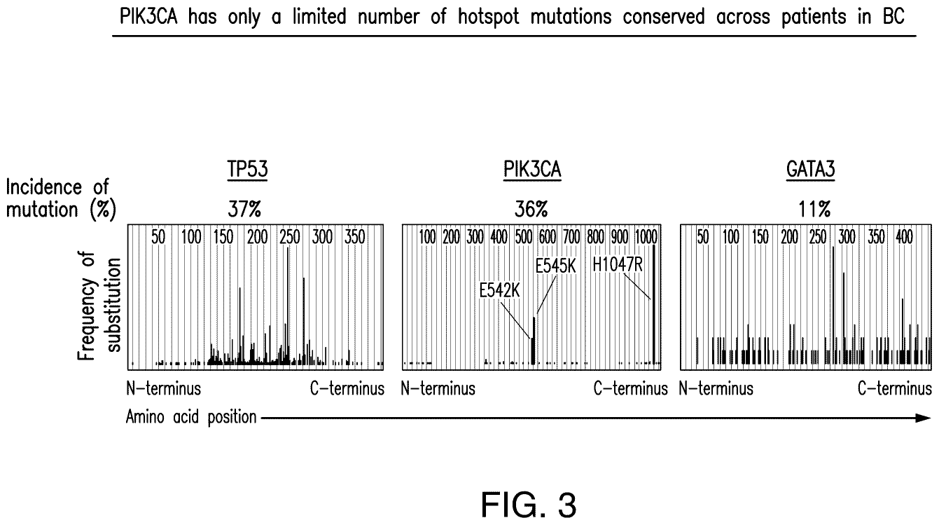

Previous study demonstrated that a third of breast cancers have PIK3CA mutations. Millis et al., JAMA Oncol. 2016; 2 (12): 1565-1573. The mutations were more common in HR+breast cancer (40-80%) and were sufficient to drive breast cancer in cells and animals. FIG. 1 shows the frequency and location of PIK3CA mutations in 1,501 MSKCC HR+/HER2-breast cancer patients, where H1047L/R mutation accounted of 35% of the cases. As shown in FIG. 2, the location of hotspot mutations in PIK3CA were similar across breast cancer subtypes. As shown in FIG. 3, PIK3CA has only a limited number of hotspot mutations conserved across patients in breast cancer. These features made targeting specific mutations of PIK3CA a promising strategy for targeting and eliminating breast cancer cells.

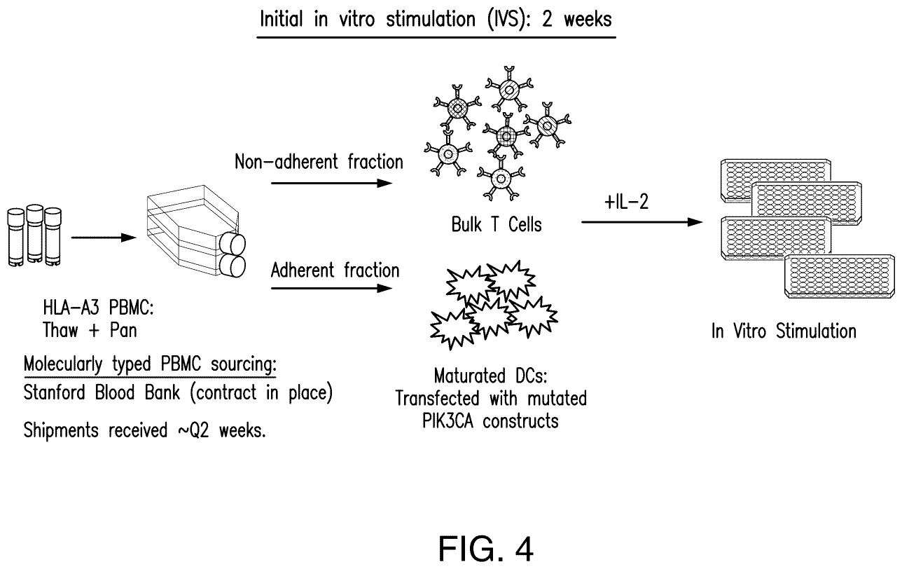

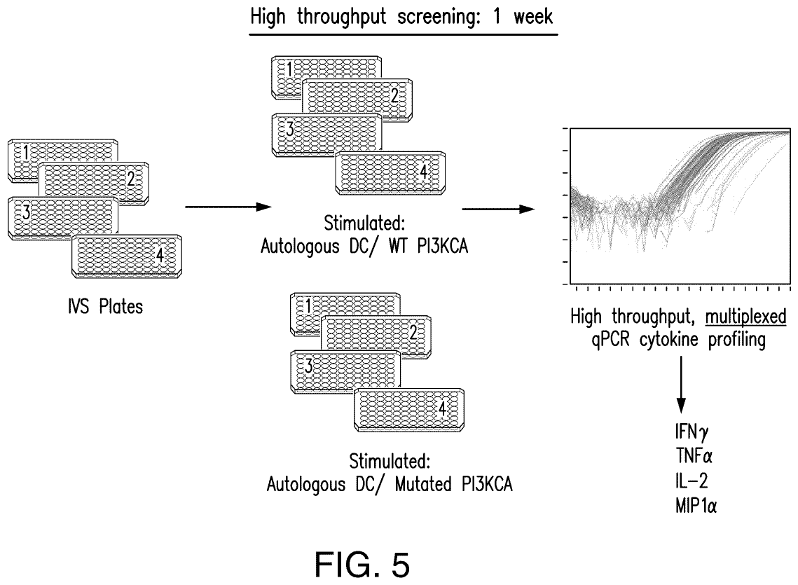

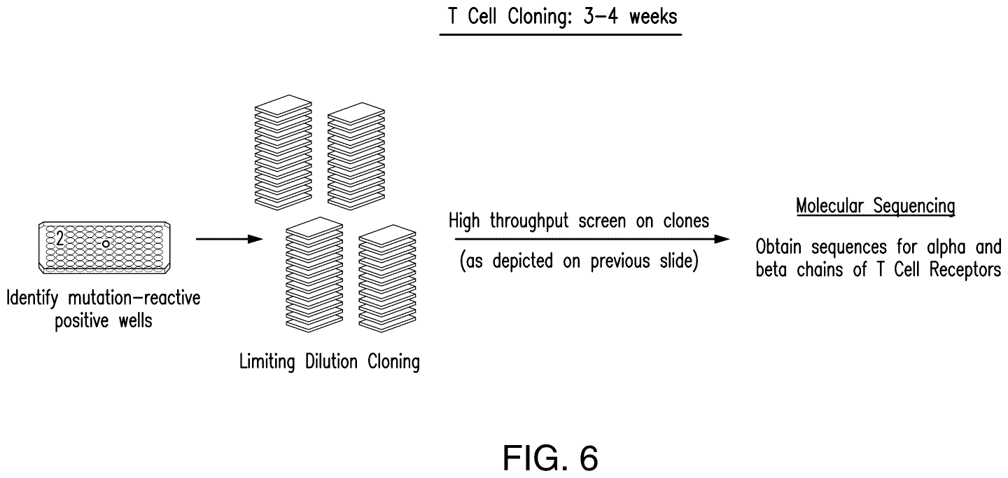

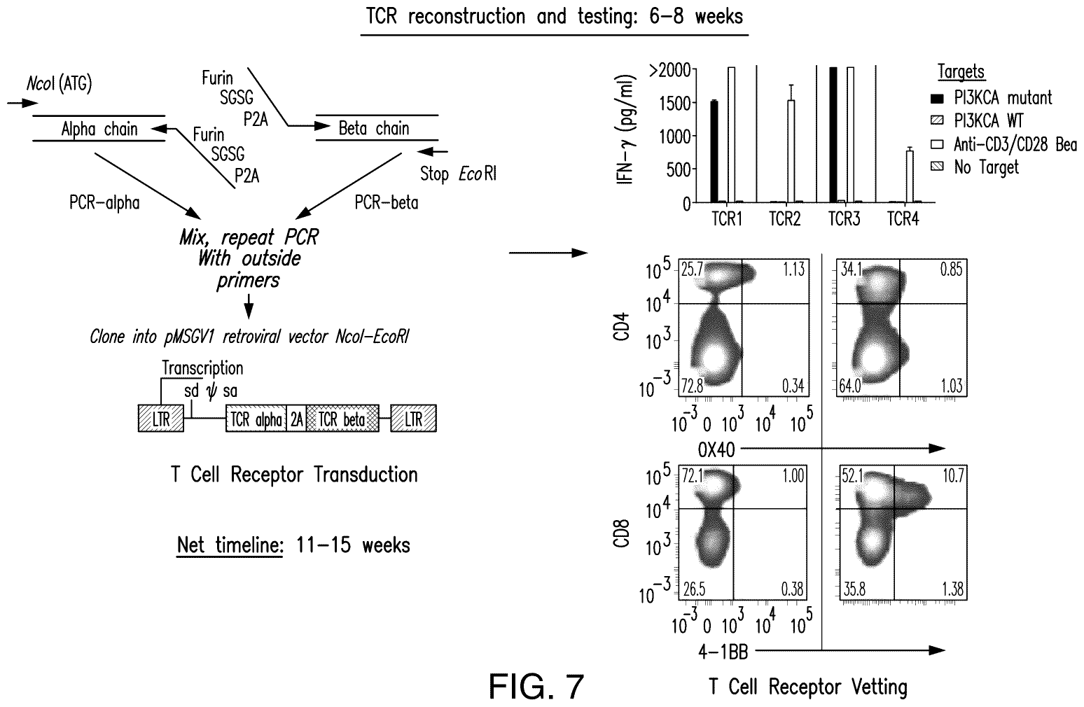

To identify TCRs specifically targeting mutant PIK3CA, peripheral blood mononuclear cells (PBMCs) from healthy donors were stimulated with autologous mature antigen-presenting cells transfected with mRNA encoding a 65-amino acid segment of the PIK3CA gene flanking common hotspot mutations. The cells received in vitro antigen stimulation two/three times over a period of two/three weeks in the presence of low-dose IL-2 (90 IU/mL) (FIG. 4). High throughput reactivity screen was then performed, where individual in vitro sensitized microwells were tested for mutation-specific reactivity by incubating with autologous APCs transfected with mRNA encoding either the wild-type (WT) or mutated PIK3CA (FIG. 5). Mutation-specific recognition is determined by the preferential upregulation of mRNA transcript of acute inflammatory markers such as IFN-g and TNF-a by high throughput quantitative PCR (FIG. 5). Mutation-specific T cells were then isolated, where positive microwells underwent limiting dilution cloning to derive T cell clones of a single specificity, and individual clones were screened for mutation-specific recognition using previously described methodology. Molecular sequencing was then performed on positive clones to derive the alpha and beta chain sequences of its T cell receptor (FIG. 6). Further, mutant PIK3CA specific TCRs were reconstruction. The genetic sequences encoding the alpha and beta chains of the mutation-specific TCR were cloned into a retroviral vector and stably integrated into allogeneic PBMC (FIG. 7). Conference of reactivity to mutation-specific transfectants and HLA-matched tumor cell line targets comprising the mutant antigen were determined by IFN-g secretion and upregulation of costimulatory molecules such as 4-1BB and OX40. A representative schema of this process is shown in FIG. 7 as an example. Stimulation and screening constructs for mPIK3CA are shown in FIG. 8.

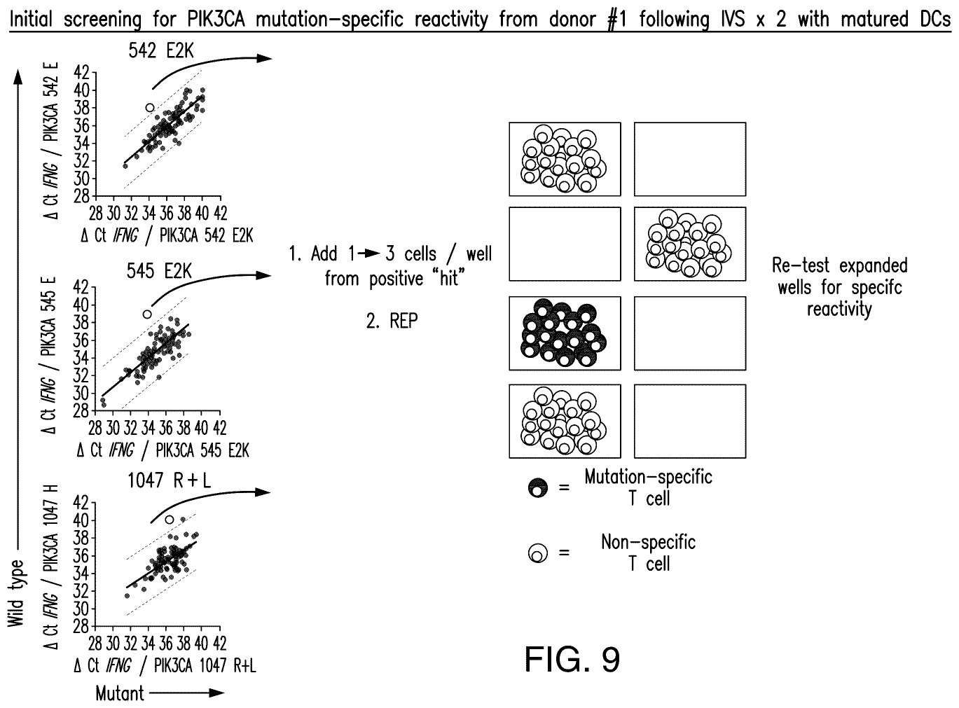

Mutation-reactive microwells were identified following in vitro sensitization. As shown in FIG. 9, delta CT values from sister wells against mutated or WT PI3KCA plotted on the X and Y axis respectively. Each dot represents an individual sensitized microwell. Mutation-specific recognition of the hotspot mutations, E542K in the top panel, E545K mutation in the middle panel and the H1047R/L in the bottom panel are also shown in FIG. 8, where outliner dots identified by the arrows indicating wells that preferentially upregulate IFN-g transcript in response to mutated PIK3CA were selected to undergo limiting dilution cloning to derive mutation-specific T cell clones. MHC Class I and Class II restricted tranfections controls, e.g., MART-1/A2: 01 (modified): ghsyttaeelagigiltvilgvlll [SEQ ID NO: 78] and MAGE-A3 DPB04: ILGDPKKLLTQHFVQE NYLEYRQVP [SEQ ID NO: 79], were used.

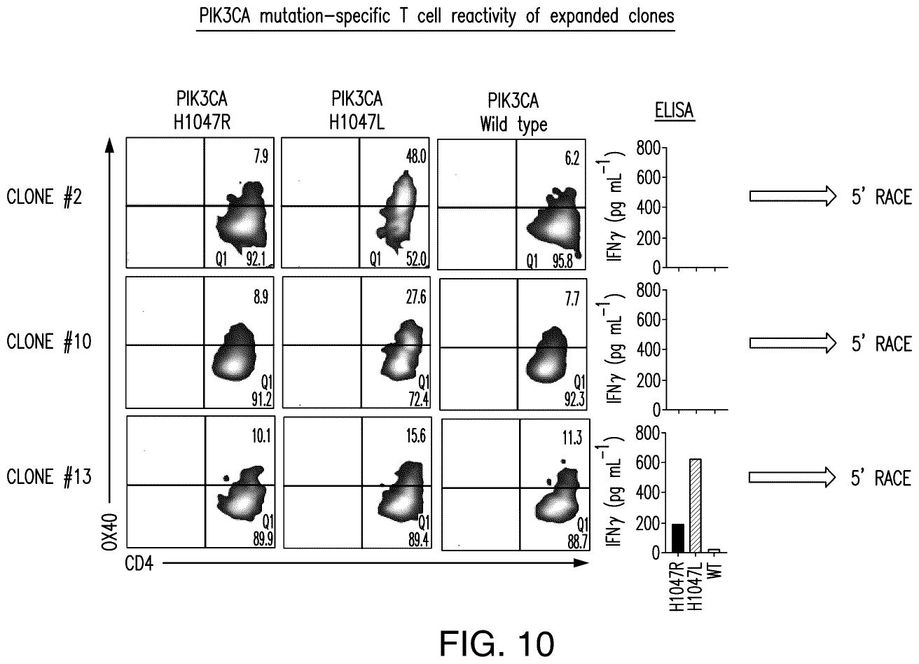

Clones derived from limiting dilution were further screened, where growth-positive wells following limiting dilution cloning were screened for PIK3CA mutation-specific recognition by measuring the preferential upregulation of the costimulatory molecule, OX40 (FIG. 10, left panel) and/or the release of IFN-g (FIG. 10, right panel) in response to the mutated antigen. Positive clones were selected to undergo molecular sequencing to derive the genetic code for the alpha and beta chains of their T cell receptor.

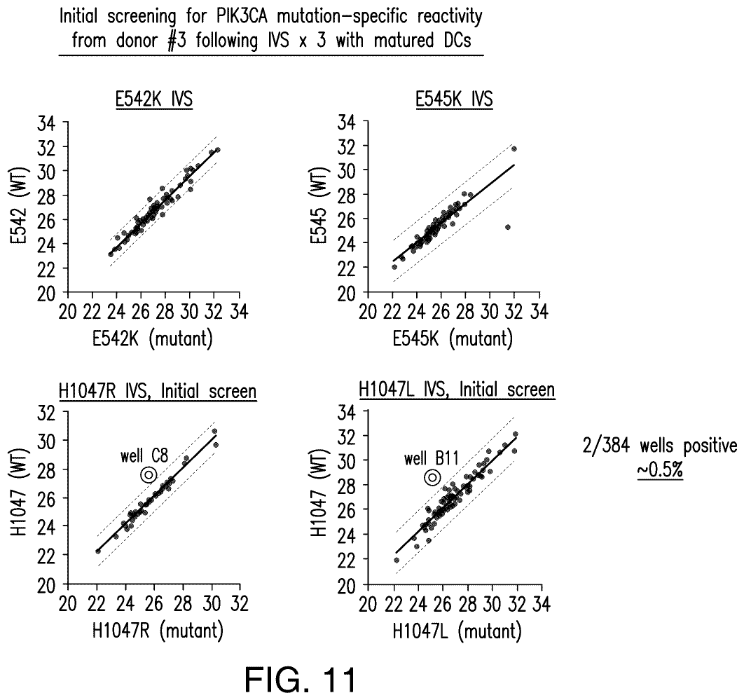

Similar screening was also performed with T cells derived from Donor 3 as show in FIG. 11. Delta CT values from paired microwells against mutant or WT PIK3CA were plotted on the X and Y axis respectively. Each dot represented an individual sensitized microwell (n=384) sensitized against one of four PI3KCA hotspot mutations listed. Well C8 (bottom left) derived from a H1047R-sensitized IVS and Well B11 (bottom right) derived from a H1047L-sensitized IVS were mutation-specific, as determined by preferential upregulation of IFN-g mRNA to mutant antigen.

Single-cell sequencing was incorporated into the discovery platform, the benefit of which includes, but are not limited to: a) potential to remove requirement to perform limited dilution cloning and save at least 2 weeks of time; b) ensuring maximal TCR diversity is captured by avoiding limited dilution cloning, which minimizes risk of losing TCRs associated with slow or poor growing T cells; c) increased discovery throughput by reduction in staff labor to tend to micro-wells and cloning and by making certain steps amenable to automation; and d) increased accuracy in identifying reactive TCRs (if paired with full RNA-seq, can design screens to incorporate assessment of both TCR frequency with function).

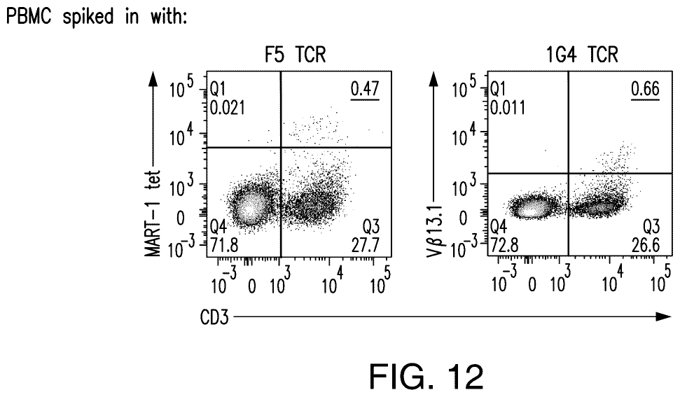

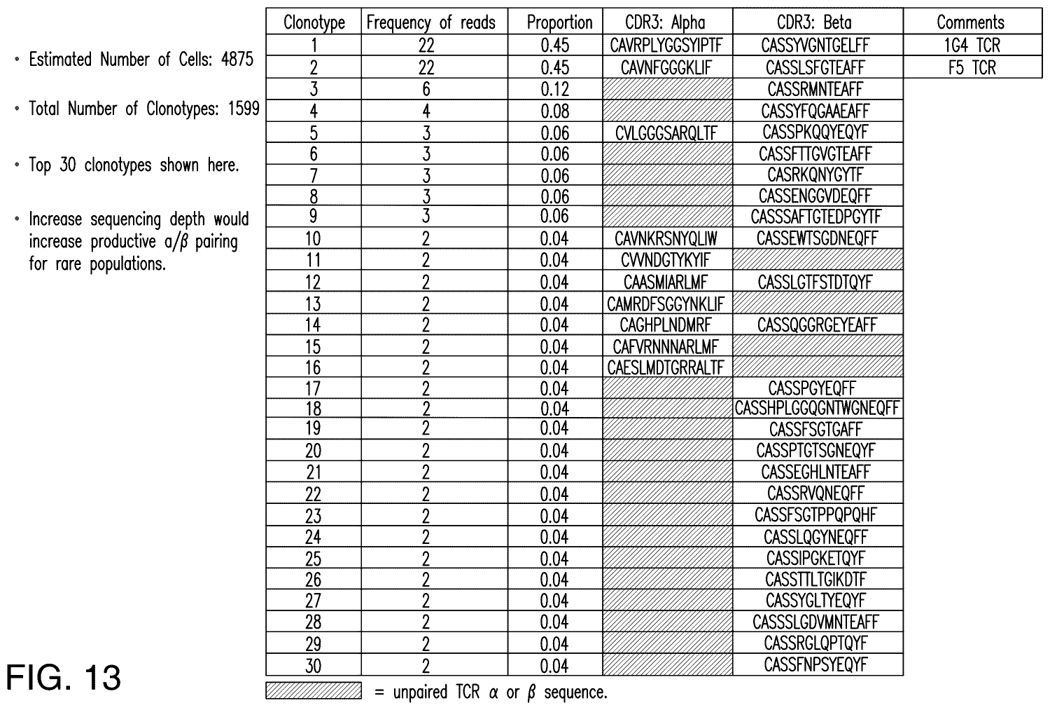

PBMCs were transduced with known TCRs (F5=MART-1, 1G4=NY-ESO-1). TCR transduced T cells were spiked at a known concentration into a bulk untransduced PBMC mixture. Single cell sequencing was used to assess whether the platform can: i) retrieve high quality and correctly paired TCR a/b sequences from the spiked samples, and ii) accurately quantify the frequency of the spiked samples (FIG. 12). FIG. 13 shows that sequencing correctly identified, paired, and quantified known TCRs within a bulk PBMC population.

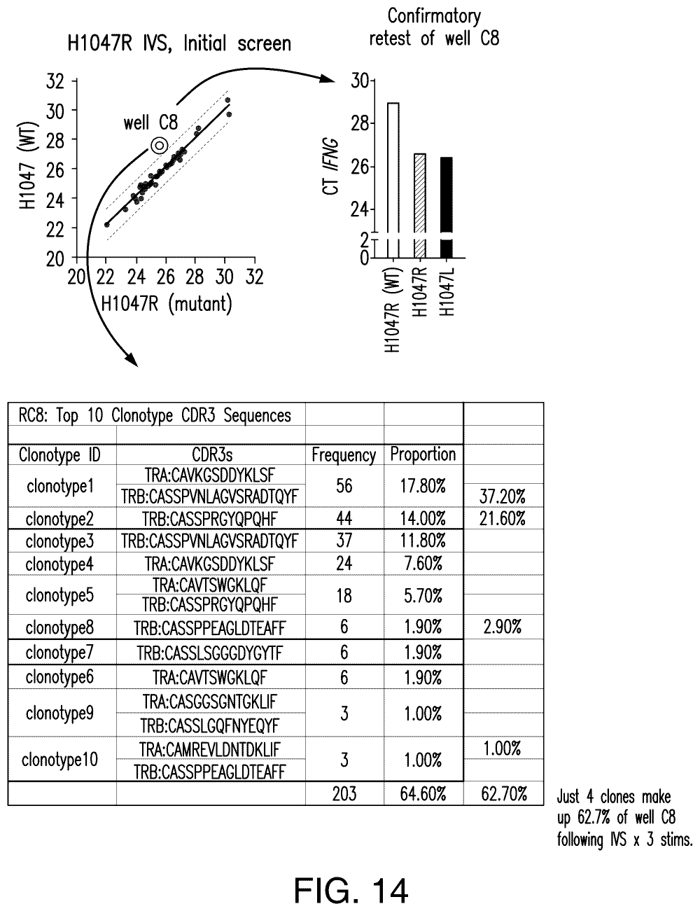

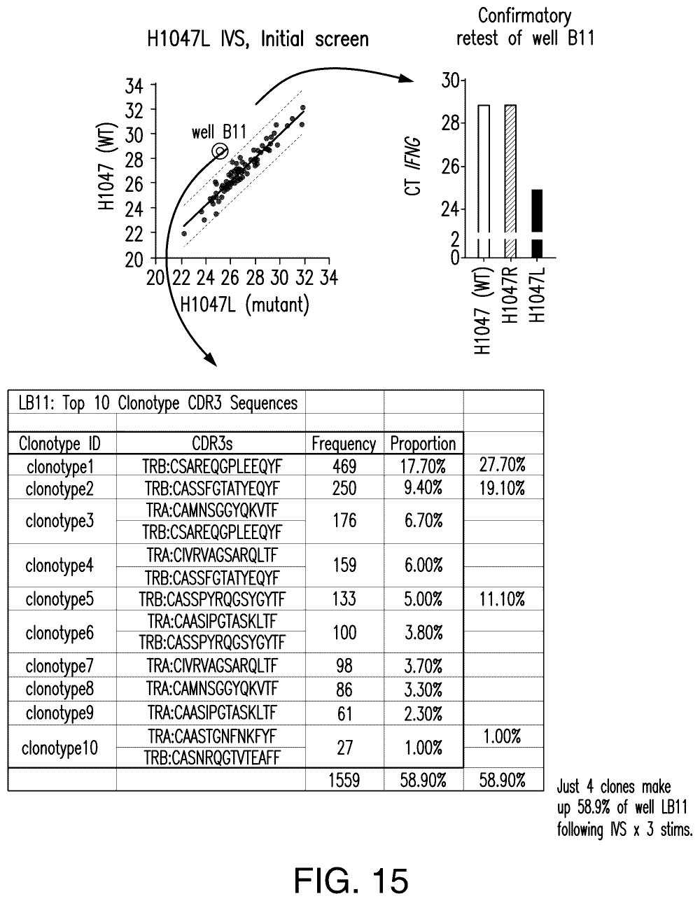

FIG. 14 shows the confirmation of RC8 reactivity. Autologous APCs transfected with RNA encoding either WT or the R/L substitutions at position 1047 in the PIK3CA gene were incubated with T cells from Well C8 (referred to as RC8). Delta CT values determined by upregulation of IFN-g transcript indicated mutation-specific recognition of both R and L hotspot mutations and no WT recognition. The table in the lower panel of FIG. 14 lists the CDR3 sequences and frequencies of the top 10 clonotypes in RC8 derived by the platform. FIG. 15 shows the confirmation of LB11 reactivity: Autologous APCs transfected with RNA encoding either WT or the R/L substitutions at position 1047 in the PIK3CA gene were incubated with T cells from Well B11 (referred to as LB11). Delta CT values determined by upregulation of IFN-g transcript indicated specific recognition of H1047L alone and no H1047R or WT recognition. The table in the lower panel of FIG. 15 lists the CDR3 sequences and frequencies of the top 10 clonotypes in LB11 derived by the platform.

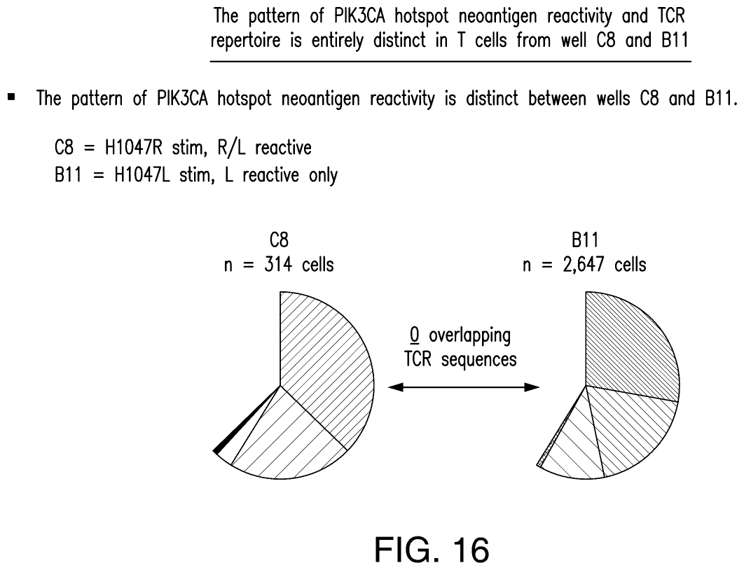

TCR sequences were unique across the reactive wells. As shown in FIG. 16, pie charts represent the clonotypes detected in Well C8 (left) versus Well B11 (right) by sequencing. Wells were highly oligoclonal and all clonotypes detected were unique to the individual wells with no shared sequences.

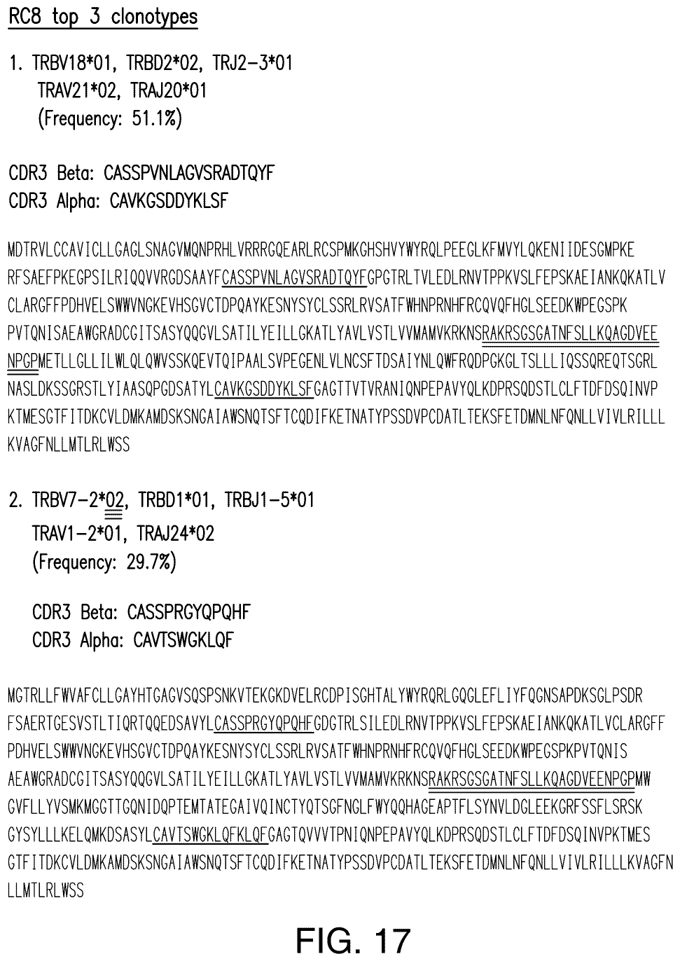

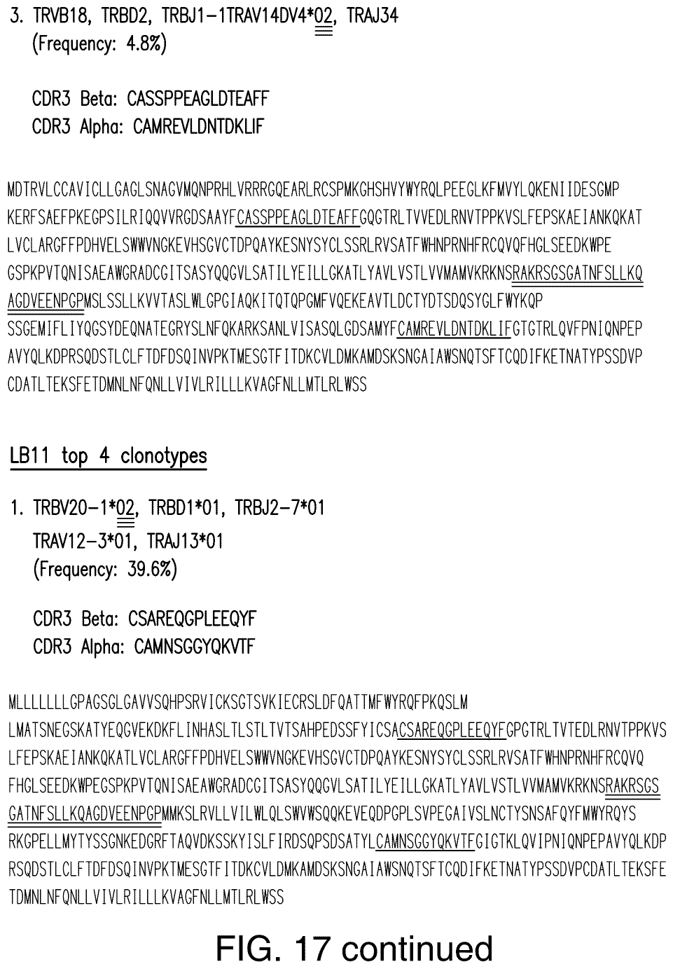

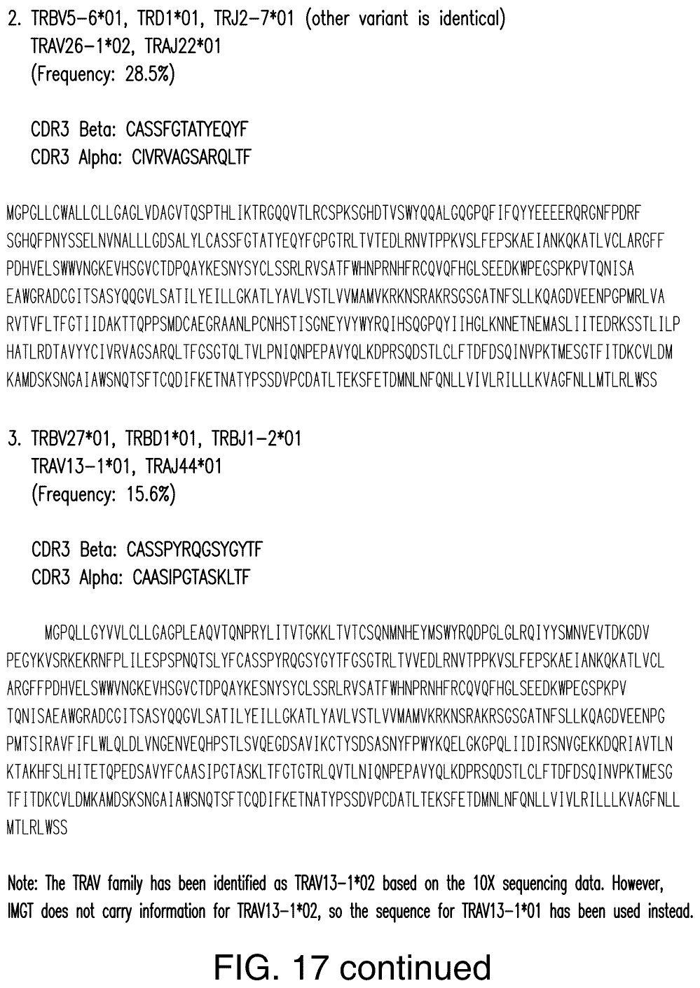

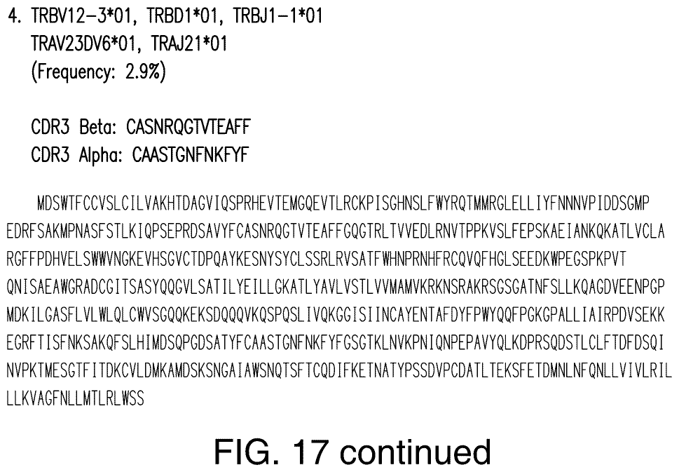

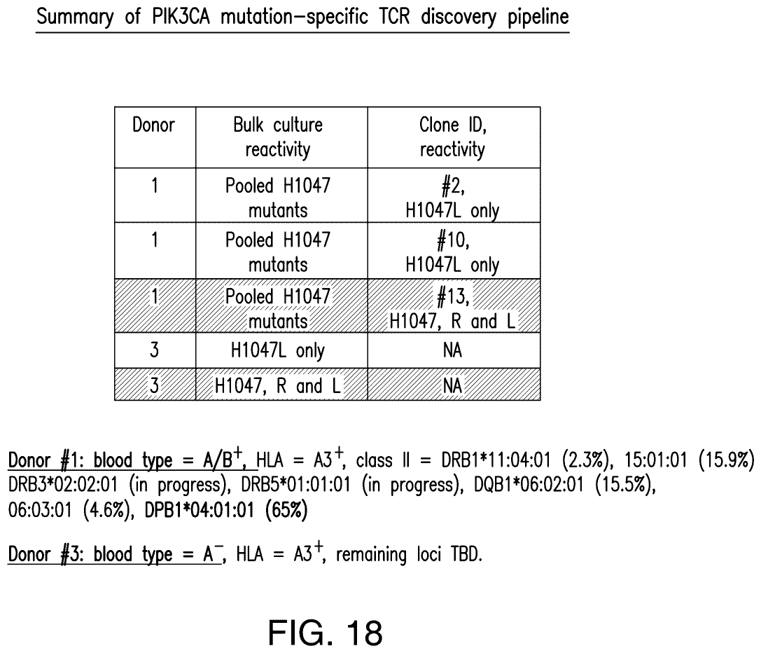

Top clonotypes of the unique TCRs were constructed into expression vectors, where the alpha chain and the beta chain of each TCR were connected by a furin-2A peptide. Based on previously described techniques, the human constant regions of the alpha and beta chains were replaced with mouse constant regions to prevent mispairing with endogenous TCR. Sequences of these TCR constructs are shown in FIG. 17 and Tables 1-7. FIG. 18 summarizes the characteristics of the PIK3CA mutation-specific TCRs.

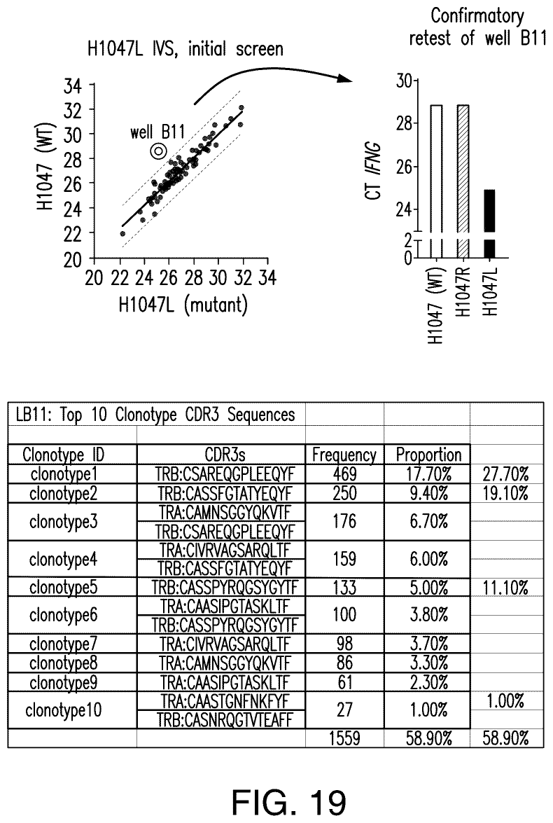

LB11 reactivity was confirmed as shown in FIG. 19. Autologous APCs transfected with RNA encoding either WT or the R/L substitutions at position 1047 in the PIK3CA gene were incubated with T cells from Well B11 (referred to as LB11). Delta CT values determined by upregulation of IFN-g transcript indicate specific recognition of H1047L alone and no H1047R or WT recognition. The top 4 clonotypes derived from sequencing were selected for TCR reconstruction and testing.

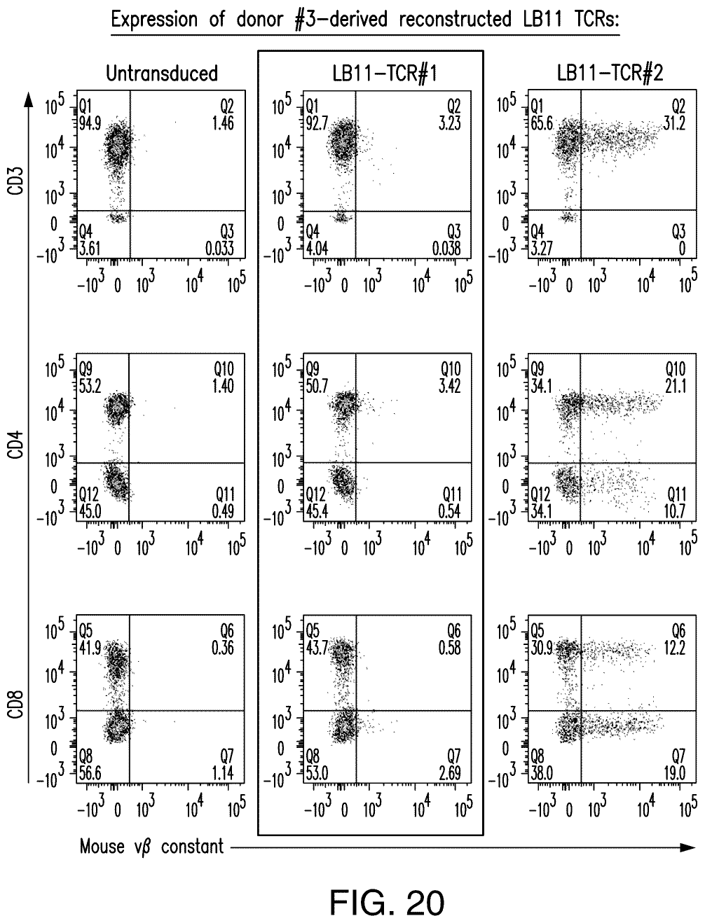

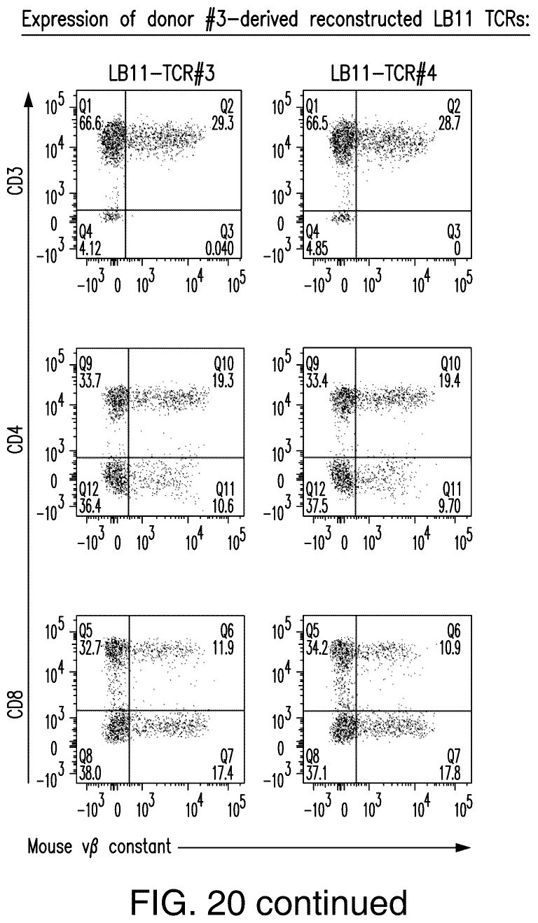

The efficiency of transduction of reconstructed LB11 TCRs in donor PBMCs is shown in FIG. 20. Allogeneic donor PBMC were transduced with retroviral particles carrying the molecular sequences for the alpha and beta chains of the LB11-derived TCR #1-4. Efficiency of transduction was determined by frequency of anti-mouse TCRβ constant staining in CD3⁺ (top), CD4⁺ (middle) and CD8⁺ (bottom) T cells at 4 days post-transduction.

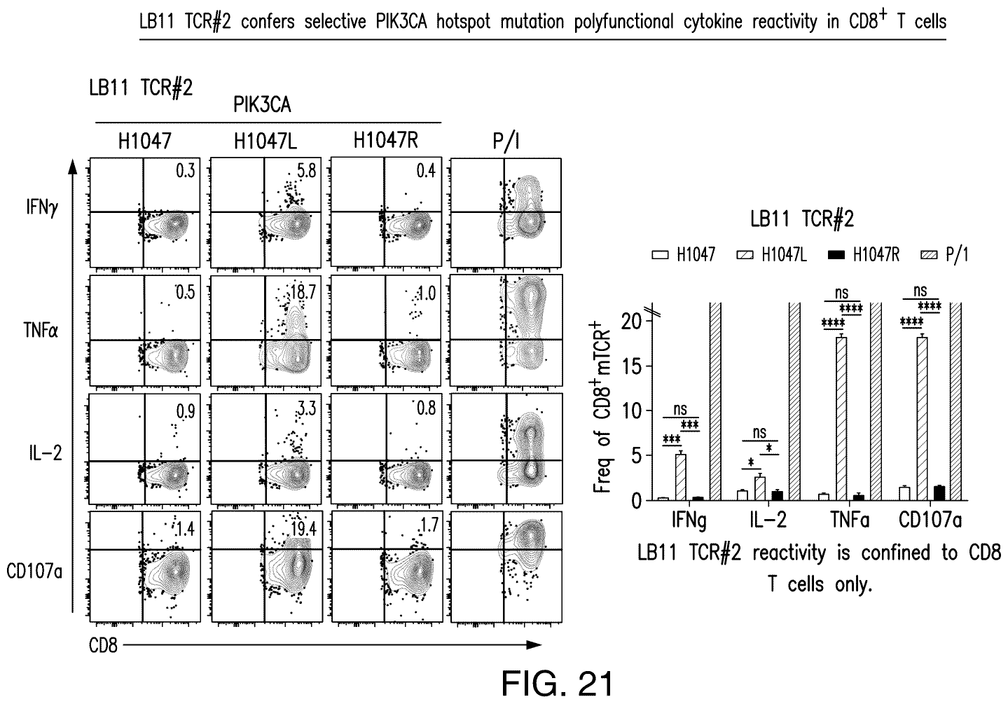

The conference of mutation-specific recognition by LB11 TCR #2 was confirmed as shown in FIG. 21. Donor PBMC with stably integrated LB11 TCR #2 were cocultured with dendritic cells expressing either H1047 WT, H1047L or H1047R mutants. Dot plots (left) and bar graph summary (right) from intracellular cytokine staining of the inflammatory markers, IFNγ, TNFα, IL-2 and CD107a show mutation-specific recognition of H1047L by transduced CD8⁺ cells. PMA-Ionomycin bypassing TCR-mediated stimulation was included as a positive control.

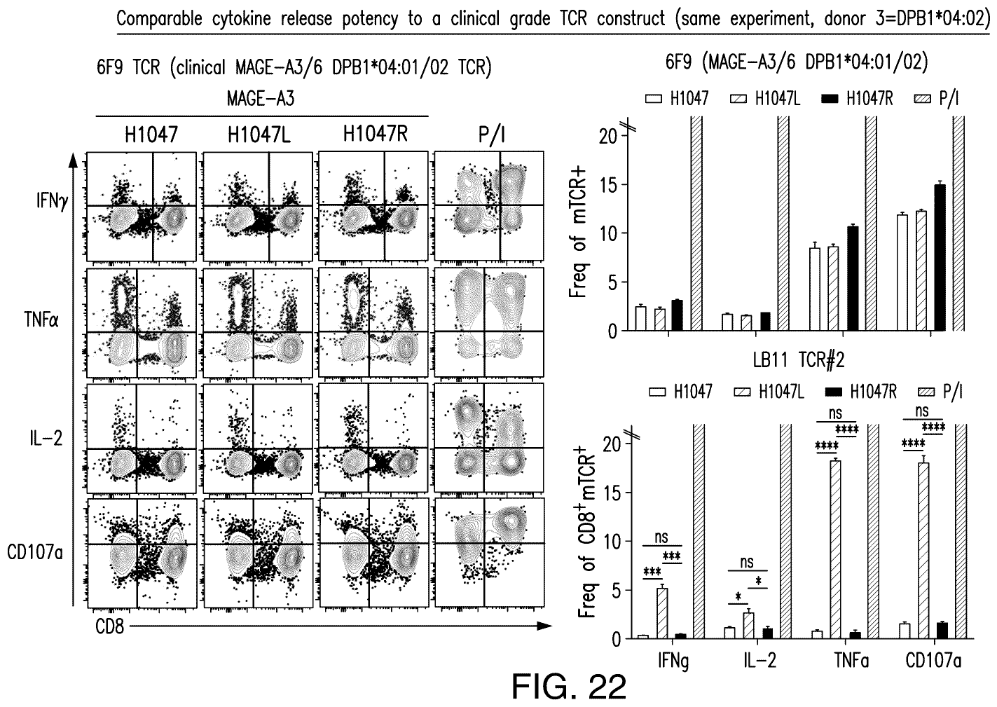

Comparable cytokine production in a clinical grade TCR against a cancer germline antigen was analyzed as shown in FIG. 22. Donor PBMCs were stably integrated with the cognate antigen, MAGE-A3. Dot plots (left) from intracellular cytokine staining of the inflammatory cytokines, IFNγ, TNFα, IL-2 and CD107a showed recognition of MAGE-A3 by transduced CD8⁺ cells. PMA-Ionomycin bypassing TCR-mediated stimulation was included as a positive control. Bar graph summary on the right comparing the cytokine-producing frequencies of mTCR+ cells in 6F9-transduced and LB11 TCR #2-transduced cells.

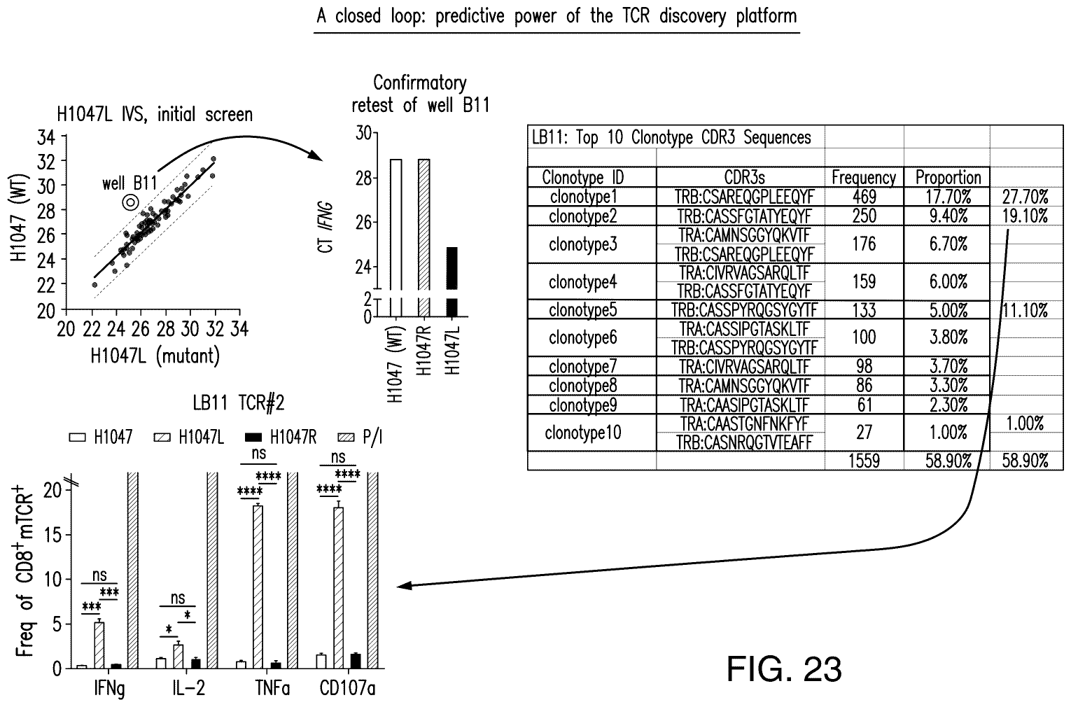

A summary of a closed loop from in vitro sensitization to TCR discovery is shown in FIG. 23. Healthy donor PBMCs sourced for mutation-specific TCRs allowed detection of a rare event ( 1/96). Sequencing rapidly determined the top clonotypes in the positive well. Synthetic reconstruction and testing of the TCR conclusively isolated a mutation-specific TCR, thus closing the loop from sensitization to identification to isolation of a TCR against a driver mutation.

### Example 2: Confirmation of PIK3CA-targeted TCR, LB11-2

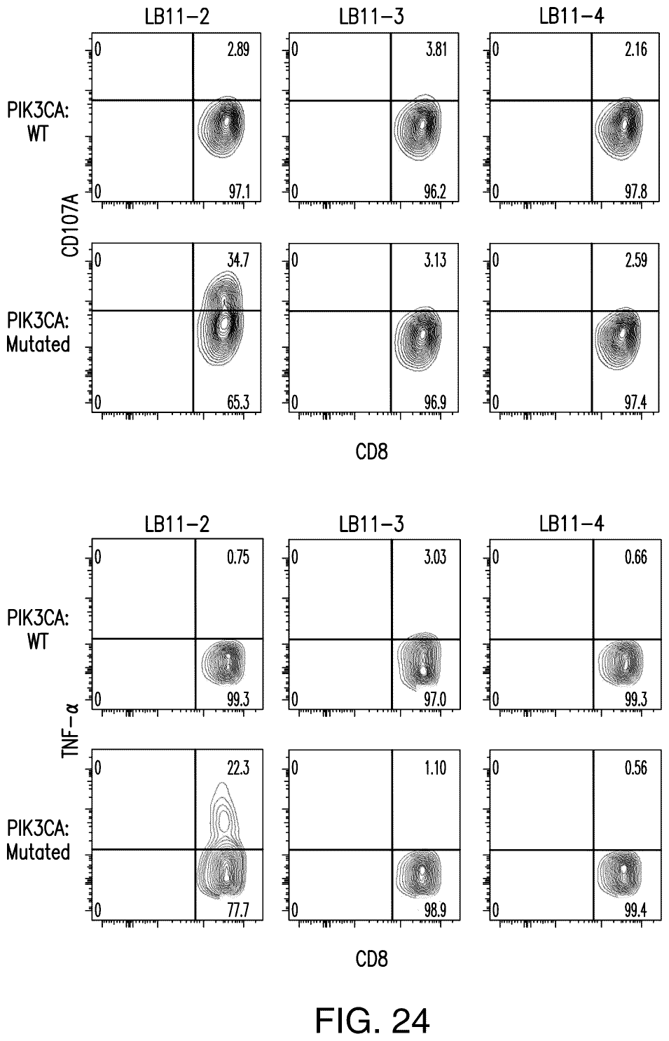

This example further characterizes mutant PIK3CA-targeted TCR, LB11-2. As shown in FIG. 24, mutation-specific reactivity of TCRs identified via VDJ sequencing from well LB11 were further tested. The genetic sequences encoding the alpha and beta chains of mutation-specific TCR derived from VDJ sequencing were cloned into a retroviral vector and stably integrated into allogeneic donor cells. The 3 unique TCR sequences tested were LB11-2, LB11-3 and LB11-4. TCR-transduced cells were cocultured with autologous dendritic cells (DC) that had been transiently transfected with RNA encoding either wildtype or mutant PIK3CA. Reactivity was determined by observing the production of the degranulation marker, CD107A, (upper panels) or the inflammatory cytokine, TNF-α, (lower panels) in response to the DC expressing mutated PIK3CA. LB11-2 was the only mutation-specific TCR identified within this set.

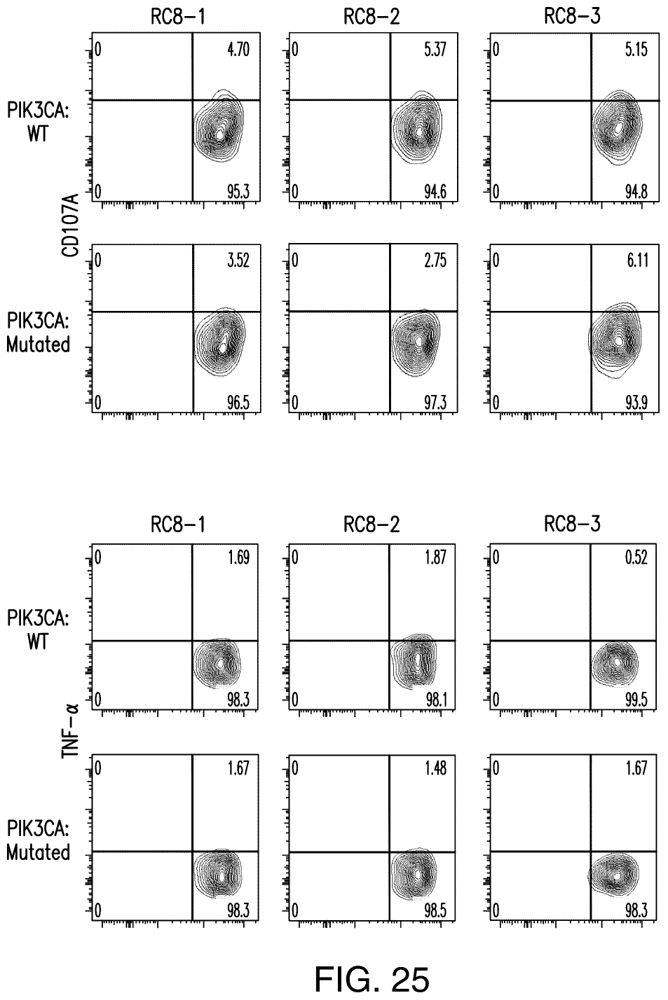

Similarly, FIG. 25 shows testing of mutation-specific reactivity of TCRs identified via VDJ sequencing from well RC8 using the same method. None of the three TCRs derived from well RC8 were identified to be mutation-specific.

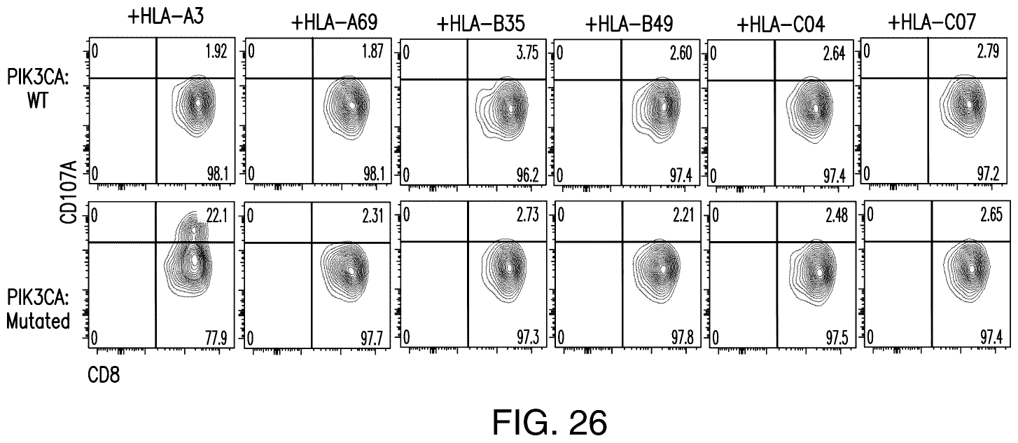

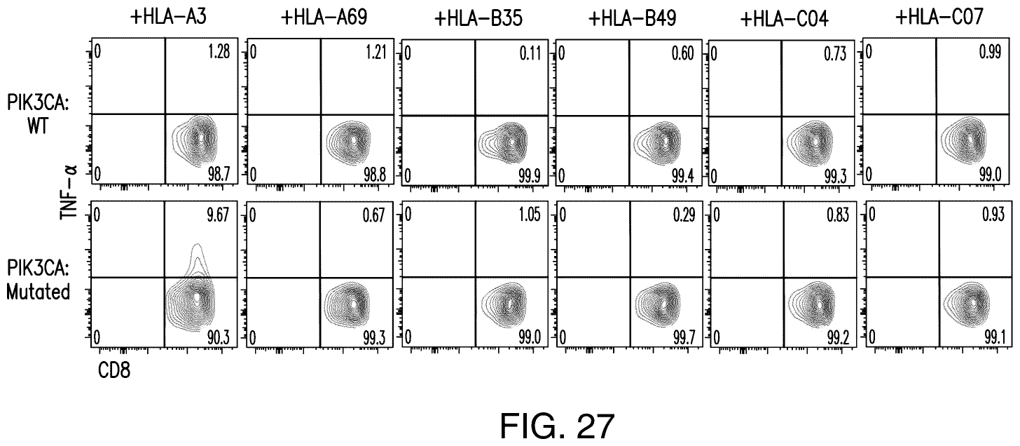

Furthermore, HLA-A3 was identified as the restriction element for PIK3CA-mutation specific TCR, LB11-2. LB11-2 TCR were retrovirally integrated into the genome of healthy donor T cells. At day 4-6 post-transduction, T cells were incubated with monkey-derived antigen-presenting cells (COS-7) that had been co-electroporated with RNA encoding a single Class IHLA allele, and either the wildtype or mutated PIK3CA RNA. The mutated version comprised a histidine to leucine substitution at position 1047 (H1047L). Cells were gated on CD8⁺ TCR+expression. As shown in FIGS. 26 and 27, changes in production of the degranulation protein, CD107A and the inflammatory cytokine, TNF-α, respectively, were used to indicate the presence of mutation-specific reactivity in the context of the right HLA allele. The data showed that LB11-2 recognized mutated H1047L in the context of HLA-A3.

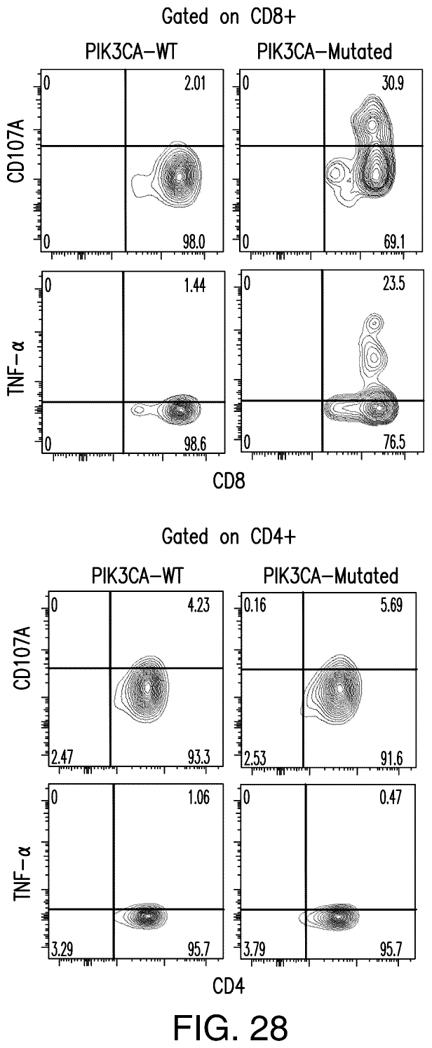

Additionally, FIG. 28 shows that PIK3CA-mutant specific TCR, LB11-2, was co-receptor-dependent. LB11-2 TCR were retrovirally integrated into the genome of healthy donor T cells. At day 4-6 post-transduction, T cells were incubated with monkey-derived antigen-presenting cells (COS-7) that had been co-electroporated with RNA encoding HLA-A3, and either the wildtype or mutated PIK3CA RNA. Cells were gated on CD8⁺ TCR+ (upper panels) or CD4⁺ TCR+ (lower panels) expression. A change in production of the CD107A and TNF-α, was used to indicate the presence of mutation-specific reactivity. The data demonstrated that LB11-2 was not functional within the CD4⁺ T cell, and required the presence of the CD8 coreceptor in order to recognize mutated H1047L.

The sequence of LB11-2 were identified, which comprises: TRBV5-6*01, TRD1*01, TRJ2-7*01, TRAV26-1*02, and TRAJ22*01. The alpha chain variable region comprised SEQ ID NO: 47, and the beta chain variable region comprised SEQ ID NO: 48.

From the foregoing description, it will be apparent that variations and modifications may be made to the invention described herein to adopt it to various usages and conditions. Such embodiments are also within the scope of the following claims.

All patents and publications and sequences referred to by accession or reference number mentioned in this specification are herein incorporated by reference to the same extent as if each independent patent and publication and sequence was specifically and individually indicated to be incorporated by reference.

What is claimed is:

1. A T cell receptor (TCR) specifically targeting a PIK3CA peptide, wherein the PIK3CA peptide comprises a mutation.

2. The TCR of claim 1, wherein the mutation is selected from the group consisting of H104R, E545K, E542K, N345K, H1047L, E726K, C420R and any combination thereof.

3. The TCR of claim 2, wherein the mutation is H1047R or H1047L.

4. The TCR of any one of claims 1-3, comprising an extracellular domain, a transmembrane domain and an intracellular domain, wherein the extracellular domain binds to the PIK3CA peptide.

5. A T cell receptor (TCR) of any one of claims 1-4, comprising an extracellular domain that comprises:
a) an α chain variable region CDR3 comprising the amino acid sequence set forth in SEQ ID NO: 3 or a conservative modification thereof, and a β chain variable region CDR3 comprising the amino acid sequence set forth in SEQ ID NO: 6 or a conservative modification thereof;
b) an α chain variable region CDR3 comprising the amino acid sequence set forth in SEQ ID NO: 13 or a conservative modification thereof, and a β chain variable region CDR3 comprising the amino acid sequence set forth in SEQ ID NO: 16 or a conservative modification thereof;
c) an α chain variable region CDR3 comprising the amino acid sequence set forth in SEQ ID NO: 23 or a conservative modification thereof, and a β chain variable region CDR3 comprising the amino acid sequence set forth in SEQ ID NO: 26 or a conservative modification thereof;
d) an α chain variable region CDR3 comprising the amino acid sequence set forth in SEQ ID NO: 33 or a conservative modification thereof, and a β chain variable region CDR3 comprising the amino acid sequence set forth in SEQ ID NO: 36 or a conservative modification thereof;
e) an α chain variable region CDR3 comprising the amino acid sequence set forth in SEQ ID NO: 43 or a conservative modification thereof, and a β chain variable region CDR3 comprising the amino acid sequence set forth in SEQ ID NO: 46 or a conservative modification thereof;
f) an α chain variable region CDR3 comprising the amino acid sequence set forth in SEQ ID NO: 53 or a conservative modification thereof, and a β chain variable region CDR3 comprising the amino acid sequence set forth in SEQ ID NO: 56 or a conservative modification thereof; or
g) an α chain variable region CDR3 comprising the amino acid sequence set forth in SEQ ID NO: 63 or a conservative modification thereof, and a β chain variable region CDR3 comprising the amino acid sequence set forth in SEQ ID NO: 66 or a conservative modification thereof.

6. The TCR of claim 5, wherein the extracellular domain comprises:
a) an α chain variable region CDR2 comprising the amino acid sequence set forth in SEQ ID NO: 2 or a conservative modification thereof, and a β chain variable region CDR2 comprising the amino acid sequence set forth in SEQ ID NO: 5 or a conservative modification thereof;
b) an α chain variable region CDR2 comprising the amino acid sequence set forth in SEQ ID NO: 12 or a conservative modification thereof, and a β chain variable region CDR2 comprising the amino acid sequence set forth in SEQ ID NO: 15 or a conservative modification thereof;
c) an α chain variable region CDR2 comprising the amino acid sequence set forth in SEQ ID NO: 22 or a conservative modification thereof, and a β chain variable region CDR2 comprising the amino acid sequence set forth in SEQ ID NO: 25 or a conservative modification thereof;
d) an α chain variable region CDR2 comprising the amino acid sequence set forth in SEQ ID NO: 32 or a conservative modification thereof, and a β chain variable region CDR2 comprising the amino acid sequence set forth in SEQ ID NO: 35 or a conservative modification thereof;
e) an α chain variable region CDR2 comprising the amino acid sequence set forth in SEQ ID NO: 42 or a conservative modification thereof, and a β chain variable region CDR2 comprising the amino acid sequence set forth in SEQ ID NO: 45 or a conservative modification thereof;
f) an α chain variable region CDR2 comprising the amino acid sequence set forth in SEQ ID NO: 52 or a conservative modification thereof, and a β chain variable region CDR2 comprising the amino acid sequence set forth in SEQ ID NO: 55 or a conservative modification thereof; or
g) an α chain variable region CDR2 comprising the amino acid sequence set forth in SEQ ID NO: 62 or a conservative modification thereof, and a β chain variable region CDR2 comprising the amino acid sequence set forth in SEQ ID NO: 65 or a conservative modification thereof.

7. The TCR of claim 5 or 6, wherein the extracellular domain comprises:
a) an α chain variable region CDR1 comprising the amino acid sequence set forth in SEQ ID NO: 1 or a conservative modification thereof, and a β chain variable region CDR1 comprising the amino acid sequence set forth in SEQ ID NO: 4 or a conservative modification thereof;
b) an α chain variable region CDR1 comprising the amino acid sequence set forth in SEQ ID NO: 11 or a conservative modification thereof, and a β chain variable region CDR1 comprising the amino acid sequence set forth in SEQ ID NO: 14 or a conservative modification thereof;
c) an α chain variable region CDR1 comprising the amino acid sequence set forth in SEQ ID NO: 21 or a conservative modification thereof, and a β chain variable region CDR1 comprising the amino acid sequence set forth in SEQ ID NO: 24 or a conservative modification thereof;
d) an α chain variable region CDR1 comprising the amino acid sequence set forth in SEQ ID NO: 31 or a conservative modification thereof, and a β chain variable region CDR1 comprising the amino acid sequence set forth in SEQ ID NO: 34 or a conservative modification thereof;
e) an α chain variable region CDR1 comprising the amino acid sequence set forth in SEQ ID NO: 41 or a conservative modification thereof, and a β chain variable region CDR1 comprising the amino acid sequence set forth in SEQ ID NO: 44 or a conservative modification thereof;
f) an α chain variable region CDR1 comprising the amino acid sequence set forth in SEQ ID NO: 51 or a conservative modification thereof, and a β chain variable region CDR1 comprising the amino acid sequence set forth in SEQ ID NO: 54 or a conservative modification thereof; or
g) an α chain variable region CDR1 comprising the amino acid sequence set forth in SEQ ID NO: 61 or a conservative modification thereof, and a β chain variable region CDR1 comprising the amino acid sequence set forth in SEQ ID NO: 64 or a conservative modification thereof.

8. The TCR of any one of claims 1-7, comprising an extracellular domain that comprises:
a) an α chain variable region CDR1 comprising the amino acid sequence set forth in SEQ ID NO: 1; an α chain variable region CDR2 comprising the amino acid sequence set forth in SEQ ID NO: 2; and an α chain variable region CDR3 comprising the amino acid sequence set forth in SEQ ID NO: 3;
b) an α chain variable region CDR1 comprising the amino acid sequence set forth in SEQ ID NO: 11; an α chain variable region CDR2 comprising the amino acid sequence set forth in SEQ ID NO: 12; and an α chain variable region CDR3 comprising the amino acid sequence set forth in SEQ ID NO: 13;
c) an α chain variable region CDR1 comprising the amino acid sequence set forth in SEQ ID NO: 21; an α chain variable region CDR2 comprising the amino acid sequence set forth in SEQ ID NO: 22; and an α chain variable region CDR3 comprising the amino acid sequence set forth in SEQ ID NO: 23;
d) an α chain variable region CDR1 comprising the amino acid sequence set forth in SEQ ID NO: 31; an α chain variable region CDR2 comprising the amino acid sequence set forth in SEQ ID NO: 32; and an α chain variable region CDR3 comprising the amino acid sequence set forth in SEQ ID NO: 33;
e) an α chain variable region CDR1 comprising the amino acid sequence set forth in SEQ ID NO: 41; an α chain variable region CDR2 comprising the amino acid sequence set forth in SEQ ID NO: 42; and an α chain variable region CDR3 comprising the amino acid sequence set forth in SEQ ID NO: 43;
f) an α chain variable region CDR1 comprising the amino acid sequence set forth in SEQ ID NO: 51; an α chain variable region CDR2 comprising the amino acid sequence set forth in SEQ ID NO: 52; and an α chain variable region CDR3 comprising the amino acid sequence set forth in SEQ ID NO: 53; or
g) an α chain variable region CDR1 comprising the amino acid sequence set forth in SEQ ID NO: 61; an α chain variable region CDR2 comprising the amino acid sequence set forth in SEQ ID NO: 62; and an α chain variable region CDR3 comprising the amino acid sequence set forth in SEQ ID NO: 63.

9. The TCR of any one of claims 1-8, comprising an extracellular domain that comprises:
a) a β chain variable region CDR1 comprising the amino acid sequence set forth in SEQ ID NO: 4; a β chain variable region CDR2 comprising the amino acid sequence set forth in SEQ ID NO: 5; and a β chain variable region CDR3 comprising the amino acid sequence set forth in SEQ ID NO: 6;
b) a β chain variable region CDR1 comprising the amino acid sequence set forth in SEQ ID NO: 14; a β chain variable region CDR2 comprising the amino acid sequence set forth in SEQ ID NO: 15; and a β chain variable region CDR3 comprising the amino acid sequence set forth in SEQ ID NO: 16;
c) a β chain variable region CDR1 comprising the amino acid sequence set forth in SEQ ID NO: 24; a β chain variable region CDR2 comprising the amino acid sequence set forth in SEQ ID NO: 25; and a β chain variable region CDR3 comprising the amino acid sequence set forth in SEQ ID NO: 26;
d) a β chain variable region CDR1 comprising the amino acid sequence set forth in SEQ ID NO: 34; a β chain variable region CDR2 comprising the amino acid sequence set forth in SEQ ID NO: 35; and a β chain variable region CDR3 comprising the amino acid sequence set forth in SEQ ID NO: 36;
e) a β chain variable region CDR1 comprising the amino acid sequence set forth in SEQ ID NO: 44; a β chain variable region CDR2 comprising the amino acid sequence set forth in SEQ ID NO: 45; and a β chain variable region CDR3 comprising the amino acid sequence set forth in SEQ ID NO: 46;
f) a β chain variable region CDR1 comprising the amino acid sequence set forth in SEQ ID NO: 54; a β chain variable region CDR2 comprising the amino acid sequence set forth in SEQ ID NO: 55; and a β chain variable region CDR3 comprising the amino acid sequence set forth in SEQ ID NO: 56; or
g) a β chain variable region CDR1 comprising the amino acid sequence set forth in SEQ ID NO: 64; a β chain variable region CDR2 comprising the amino acid sequence set forth in SEQ ID NO: 65; and a β chain variable region CDR3 comprising the amino acid sequence set forth in SEQ ID NO: 66.

10. The TCR of any one of claims 1-9, comprising an extracellular domain that comprises:
a) an α chain variable region CDR1 comprising the amino acid sequence set forth in SEQ ID NO: 1; an α chain variable region CDR2 comprising the amino acid sequence set forth in SEQ ID NO: 2; an α chain variable region CDR3 comprising the amino acid sequence set forth in SEQ ID NO: 3; a β chain variable region CDR1 comprising the amino acid sequence set forth in SEQ ID NO: 4; a β chain variable region CDR2 comprising the amino acid sequence set forth in SEQ ID NO: 5 and a β chain variable region CDR3 comprising the amino acid sequence set forth in SEQ ID NO: 6
b) an α chain variable region CDR1 comprising the amino acid sequence set forth in SEQ ID NO: 11; an α chain variable region CDR2 comprising the amino acid sequence set forth in SEQ ID NO: 12; an α chain variable region CDR3 comprising the amino acid sequence set forth in SEQ ID NO: 13; a β chain variable region CDR1 comprising the amino acid sequence set forth in SEQ ID NO: 14; a β chain variable region CDR2 comprising the amino acid sequence set forth in SEQ ID NO: 15 and a β chain variable region CDR3 comprising the amino acid sequence set forth in SEQ ID NO: 16;
c) an α chain variable region CDR1 comprising the amino acid sequence set forth in SEQ ID NO: 21; an α chain variable region CDR2 comprising the amino acid sequence set forth in SEQ ID NO: 22; an α chain variable region CDR3 comprising the amino acid sequence set forth in SEQ ID NO: 23; a β chain variable region CDR1 comprising the amino acid sequence set forth in SEQ ID NO: 24; a β chain variable region CDR2 comprising the amino acid sequence set forth in SEQ ID NO: 25 and a β chain variable region CDR3 comprising the amino acid sequence set forth in SEQ ID NO: 26;
d) an α chain variable region CDR1 comprising the amino acid sequence set forth in SEQ ID NO: 31; an α chain variable region CDR2 comprising the amino acid sequence set forth in SEQ ID NO: 32; an α chain variable region CDR3 comprising the amino acid sequence set forth in SEQ ID NO: 33; a β chain variable region CDR1 comprising the amino acid sequence set forth in SEQ ID NO: 34; a β chain variable region CDR2 comprising the amino acid sequence set forth in SEQ ID NO: 35 and a β chain variable region CDR3 comprising the amino acid sequence set forth in SEQ ID NO: 36;
e) an α chain variable region CDR1 comprising the amino acid sequence set forth in SEQ ID NO: 41; an α chain variable region CDR2 comprising the amino acid sequence set forth in SEQ ID NO: 42; an α chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46;
f) an α chain variable region CDR1 comprising the amino acid sequence set forth in SEQ ID NO: 51; an α chain variable region CDR2 comprising the amino acid sequence set forth in SEQ ID NO: 52; an α chain variable region CDR3 comprising the amino acid sequence set forth in SEQ ID NO: 53; a β chain variable region CDR1 comprising the amino acid sequence set forth in SEQ ID NO: 54; a β chain variable region CDR2 comprising the amino acid sequence set forth in SEQ ID NO: 55 and a β chain variable region CDR3 comprising the amino acid sequence set forth in SEQ ID NO: 56; or
g) an α chain variable region CDR1 comprising the amino acid sequence set forth in SEQ ID NO: 61; an α chain variable region CDR2 comprising the amino acid sequence set forth in SEQ ID NO: 62; an α chain variable region CDR3 comprising the amino acid sequence set forth in SEQ ID NO: 63; a β chain variable region CDR1 comprising the amino acid sequence set forth in SEQ ID NO: 64; a β chain variable region CDR2 comprising the amino acid sequence set forth in SEQ ID NO: 65 and a β chain variable region CDR3 comprising the amino acid sequence set forth in SEQ ID NO: 66.

11. The TCR of claim 10, wherein the extracellular domain comprises an α chain variable region CDR1 comprising the amino acid sequence set forth in SEQ ID NO: 41; an α chain variable region CDR2 comprising the amino acid sequence set forth in SEQ ID NO: 42; an α chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46.

12. The TCR of any one of claims 1-11, comprising an extracellular domain that comprises an α chain variable region comprising an amino acid sequence that is at least about 80% homologous or identical to the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67.

13. The TCR of claim 12, wherein the extracellular domain comprises an α chain variable region comprising the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67.

14. The TCR of any one of claims 1-13, comprising an extracellular domain that comprises a β chain variable region comprising an amino acid sequence that is at least about 80% homologous or identical to the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68.

15. The TCR of claim 14, wherein the extracellular domain comprises a β chain variable region comprising the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68.

16. The TCR of any one of claims 1-15, comprising an extracellular domain that comprises:
a) an α chain variable region comprising an amino acid sequence that is at least about 80% homologous or identical to the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67; and
b) a β chain variable region comprising an amino acid sequence that is at least about 80% homologous or identical to the amino acid sequence set forth in SEQ ID NO: 8, SEQ ID NO: 18, SEQ ID NO: 28, SEQ ID NO: 38, SEQ ID NO: 48 SEQ ID NO: 58, or SEQ ID NO: 68.

17. The TCR of any one of claims 1-16, comprising an extracellular domain that comprises: an α chain variable region comprising the amino acid sequence set forth in SEQ ID NO: 7, SEQ ID NO: 17, SEQ ID NO: 27, SEQ ID NO: 37, SEQ ID NO: 47, SEQ ID NO: 57, or SEQ ID NO: 67; and a β chain variable region comprising the amino acid sequence set forth in SEQ ID NO: 8, SEQ ID NO: 18, SEQ ID NO: 28, SEQ ID NO: 38, SEQ ID NO: 48, SEQ ID NO: 58, or SEQ ID NO: 68.

18. The TCR of any one of claims 1-17, comprising an extracellular domain that comprises:
a) an α chain variable region comprising an amino acid sequence that is at least about 80% homologous or identical to the amino acid sequence set forth in SEQ ID NO: 7; and a β chain variable region comprising an amino acid sequence that is at least about 80% homologous or identical to the amino acid sequence set forth in SEQ ID NO: 8;
b) an α chain variable region comprising an amino acid sequence that is at least about 80% homologous or identical to the amino acid sequence set forth in SEQ ID NO: 17; and a β chain variable region comprising an amino acid sequence that is at least about 80% homologous or identical to the amino acid sequence set forth in SEQ ID NO: 18;
c) an α chain variable region comprising an amino acid sequence that is at least about 80% homologous or identical to the amino acid sequence set forth in SEQ ID NO: 27; and a β chain variable region comprising an amino acid sequence that is at least about 80% homologous or identical to the amino acid sequence set forth in SEQ ID NO: 28;
d) an α chain variable region comprising an amino acid sequence that is at least about 80% homologous or identical to the amino acid sequence set forth in SEQ ID NO: 37; and a β chain variable region comprising an amino acid sequence that is at least about 80% homologous or identical to the amino acid sequence set forth in SEQ ID NO: 38;
e) an α chain variable region comprising an amino acid sequence that is at least about 80% homologous or identical to the amino acid sequence set forth in SEQ ID NO: 47; and a β chain variable region comprising an amino acid sequence that is at least about 80% homologous or identical to the amino acid sequence set forth in SEQ ID NO: 48;
f) an α chain variable region comprising an amino acid sequence that is at least about 80% homologous or identical to the amino acid sequence set forth in SEQ ID NO: 57; and a β chain variable region comprising an amino acid sequence that is at least about 80% homologous or identical to the amino acid sequence set forth in SEQ ID NO: 58; or
g) an α chain variable region comprising an amino acid sequence that is at least about 80% homologous or identical to the amino acid sequence set forth in SEQ ID NO: 67; and a β chain variable region comprising an amino acid sequence that is at least about 80% homologous or identical to the amino acid sequence set forth in SEQ ID NO: 68.

19. The TCR of any one of claims 1-18, comprising an extracellular domain that comprises:
a) an α chain variable region comprising the amino acid sequence set forth in SEQ ID NO: 7, and a β chain variable region comprising the amino acid sequence set forth in SEQ ID NO: 8;
b) an α chain variable region comprising the amino acid sequence set forth in SEQ ID NO: 17, and a β chain variable region comprising the amino acid sequence set forth in SEQ ID NO: 18;
c) an α chain variable region comprising the amino acid sequence set forth in SEQ ID NO: 27, and a β chain variable region comprising the amino acid sequence set forth in SEQ ID NO: 28;
d) an α chain variable region comprising the amino acid sequence set forth in SEQ ID NO: 37, and a β chain variable region comprising the amino acid sequence set forth in SEQ ID NO: 38;
e) an α chain variable region comprising the amino acid sequence set forth in SEQ ID NO: 47, and a β chain variable region comprising the amino acid sequence set forth in SEQ ID NO: 48; or
f) an α chain variable region comprising the amino acid sequence set forth in SEQ ID NO: 57, and a β chain variable region comprising the amino acid sequence set forth in SEQ ID NO: 58; or
g) an α chain variable region comprising the amino acid sequence set forth in SEQ ID NO: 67, and a β chain variable region comprising the amino acid sequence set forth in SEQ ID NO: 68.

20. The TCR of claim 19, wherein the extracellular domain comprises an α chain variable region comprising the amino acid sequence set forth in SEQ ID NO: 47 and a β chain variable region comprising the amino acid sequence set forth in SEQ ID NO: 48.

21. The TCR of any one of claims 1-20, comprising an extracellular domain that comprises an α chain comprising the amino acid sequence set forth in SEQ ID NO: 9, SEQ ID NO: 19, SEQ ID NO: 29, SEQ ID NO: 39, SEQ ID NO: 49, SEQ ID NO: 59, or SEQ ID NO: 69; and a β chain comprising the amino acid sequence set forth in SEQ ID NO: 10, SEQ ID NO: 20, SEQ ID NO: 30, SEQ ID NO: 40, SEQ ID NO: 50, SEQ ID NO: 60, or SEQ ID NO: 70.

22. The TCR of claim 21, wherein the extracellular domain comprises an α chain comprising the amino acid sequence set forth in SEQ ID NO: 49 and a β chain comprising the amino acid sequence set forth in SEQ ID NO: 50.

23. The TCR of any one of claims 1-4, comprising an extracellular domain that binds to the same epitope on a human mutant PIK3CA polypeptide as a reference TCR or a functional fragment thereof, wherein the reference TCR or a functional fragment thereof comprises:
a) an α chain variable region CDR1 comprising the amino acid sequence set forth in SEQ ID NO: 1; an α chain variable region CDR2 comprising the amino acid sequence set forth in SEQ ID NO: 2; an α chain variable region CDR3 comprising the amino acid sequence set forth in SEQ ID NO: 3; a β chain variable region CDR1 comprising the amino acid sequence set forth in SEQ ID NO: 4; a β chain variable region CDR2 comprising the amino acid sequence set forth in SEQ ID NO: 5 and a β chain variable region CDR3 comprising the amino acid sequence set forth in SEQ ID NO: 6;
b) an α chain variable region CDR1 comprising the amino acid sequence set forth in SEQ ID NO: 11; an α chain variable region CDR2 comprising the amino acid sequence set forth in SEQ ID NO: 12; an α chain variable region CDR3 comprising the amino acid sequence set forth in SEQ ID NO: 13; a β chain variable region CDR1 comprising the amino acid sequence set forth in SEQ ID NO: 14; a β chain variable region CDR2 comprising the amino acid sequence set forth in SEQ ID NO: 15 and a β chain variable region CDR3 comprising the amino acid sequence set forth in SEQ ID NO: 16;
c) an α chain variable region CDR1 comprising the amino acid sequence set forth in SEQ ID NO: 21; an α chain variable region CDR2 comprising the amino acid sequence set forth in SEQ ID NO: 22; an α chain variable region CDR3 comprising the amino acid sequence set forth in SEQ ID NO: 23; a β chain variable region CDR1 comprising the amino acid sequence set forth in SEQ ID NO: 24; a β chain variable region CDR2 comprising the amino acid sequence set forth in SEQ ID NO: 25 and a β chain variable region CDR3 comprising the amino acid sequence set forth in SEQ ID NO: 26;
d) an α chain variable region CDR1 comprising the amino acid sequence set forth in SEQ ID NO: 31; an α chain variable region CDR2 comprising the amino acid sequence set forth in SEQ ID NO: 32; an α chain variable region CDR3 comprising the amino acid sequence set forth in SEQ ID NO: 33; a β chain variable region CDR1 comprising the amino acid sequence set forth in SEQ ID NO: 34; a β chain variable region CDR2 comprising the amino acid sequence set forth in SEQ ID NO: 35 and a β chain variable region CDR3 comprising the amino acid sequence set forth in SEQ ID NO: 36;
e) an α chain variable region CDR1 comprising the amino acid sequence set forth in SEQ ID NO: 41; an α chain variable region CDR2 comprising the amino acid sequence set forth in SEQ ID NO: 42; an α chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46;
f) an α chain variable region CDR1 comprising the amino acid sequence set forth in SEQ ID NO: 51; an α chain variable region CDR2 comprising the amino acid sequence set forth in SEQ ID NO: 52; an α chain variable region CDR3 comprising the amino acid sequence set forth in SEQ ID NO: 53; a β chain variable region CDR1 comprising the amino acid sequence set forth in SEQ ID NO: 54; a β chain variable region CDR2 comprising the amino acid sequence set forth in SEQ ID NO: 55 and a β chain variable region CDR3 comprising the amino acid sequence set forth in SEQ ID NO: 56; or
g) an α chain variable region CDR1 comprising the amino acid sequence set forth in SEQ ID NO: 61; an α chain variable region CDR2 comprising the amino acid sequence set forth in SEQ ID NO: 62; an α chain variable region CDR3 comprising the amino acid sequence set forth in SEQ ID NO: 63; a β chain variable region CDR1 comprising the amino acid sequence set forth in SEQ ID NO: 64; a β chain variable region CDR2 comprising the amino acid sequence set forth in SEQ ID NO: 65 and a β chain variable region CDR3 comprising the amino acid sequence set forth in SEQ ID NO: 66.

24. The TCR of claim 23, wherein the reference TCR or a functional fragment thereof comprises an α chain variable region CDR1 comprising the amino acid sequence set forth in SEQ ID NO: 41; an α chain variable region CDR2 comprising the amino acid sequence set forth in SEQ ID NO: 42; an α chain variable region CDR3 comprising the amino acid sequence set forth in SEQ ID NO: 43; a β chain variable region CDR1 comprising the amino acid sequence set forth in SEQ ID NO: 44; a β chain variable region CDR2 comprising the amino acid sequence set forth in SEQ ID NO: 45 and a β chain variable region CDR3 comprising the amino acid sequence set forth in SEQ ID NO: 46.

25. The TCR of any one of claims 1-24, wherein the TCR is recombinantly expressed, and/or expressed from a vector.

26. The TCR of any one of claims 1-25, wherein the TCR does not target a wildtype PIK3CA peptide.

27. The TCR of any one of claims 1-26, wherein the TCR comprises a modified α-chain constant region and/or a modified β-chain constant region.

28. The TCR of claim 27, wherein the modified α-chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 80 or 81.

29. The TCR of claim 28, wherein the modified α-chain constant region comprises an amino acid sequence set forth in SEQ ID NO: 80.

30. The TCR of claim 27, wherein the modified β-chain constant region comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 82 or 83.

31. The TCR of claim 30, wherein the modified β-chain constant region comprises an amino acid sequence set forth in SEQ ID NO: 82.

32. An isolated immunoresponsive cell comprising the TCR of any one of the preceding claims.

33. The isolated immunoresponsive cell of claim 32, wherein the immunoresponsive cell is transduced with the TCR.

34. The isolated immunoresponsive cell of claim 32 or 33, wherein the TCR is constitutively expressed on the surface of the immunoresponsive cell.

35. The isolated immunoresponsive cell of any one of claims 32-34, wherein the immunoresponsive cell is selected from the group consisting of a T cell, a Natural Killer (NK) cell, a human embryonic stem cell, a lymphoid progenitor cell, a T cell-precursor cell, and a pluripotent stem cell from which lymphoid cells may be differentiated.

36. The isolated immunoresponsive cell of claim 35, wherein the immunoresponsive cell is a T cell.

37. The isolated immunoresponsive cell of claim 36, wherein the T cell is selected from the group consisting of a cytotoxic T lymphocyte (CTL), a regulatory T cell, and central memory T cells.

38. A composition comprising the immunoresponsive cell of any one of claims 32-37.

39. The composition of claim 38, which is a pharmaceutical composition comprising a pharmaceutically acceptable carrier.

40. An isolated nucleic acid molecule encoding the T cell receptor (TCR) of any one of claims 1-31.

41. A vector comprising the isolated nucleic acid molecule of claim 40.

42. The vector of claim 41, wherein the vector is a γ-retroviral vector.

43. A host cell comprising the nucleic acid molecule of claim 40.

44. The host cell of claim 43 wherein the host cell is a T cell.

45. A method for producing an immunoresponsive cell that binds to a human mutant PIK3CA peptide, comprising introducing into the immunoresponsive cell a nucleic acid sequence that encodes the TCR of any one of claims 1-31.

46. A method of treating and/or preventing a malignancy comprising a PIK3CA mutation in a subject, comprising administering to the subject an effective amount of the immunoresponsive cell of any one of claims 32-37.

47. The method of claim 46, wherein the malignancy is selected from the group consisting of breast cancer, endometrial cancer, cervical cancer, anal cancer, bladder cancer, colorectal cancer, head and neck squamous cell carcinoma, nonmelanoma skin cancer and salivary gland cancer.

48. The method of claim 47, wherein the malignancy is breast cancer.

49. The method of any one of claims 46-48, wherein the method reduces or eradicates the tumor burden in the subject.

50. The method of any one of claims 46-49, wherein the subject is a human.

51. A kit for treating and/or preventing a malignancy comprising a PIK3CA mutation, comprising the immunoresponsive cell of any one of claims 32-37, the isolated nucleic acid molecule of claim 40 or the vector of claim 41 or 42, optionally the kit further comprises written instructions for using the immunoresponsive cell for treating a subject having a malignancy.

52. The kit of claim 51, wherein the malignancy is selected from the group consisting of breast cancer, breast cancer, endometrial cancer, cervical cancer, anal cancer, bladder cancer, colorectal cancer, head and neck squamous cell carcinoma, nonmelanoma skin cancer and salivary gland cancer.

53. The kit of claim 52, wherein the malignancy is breast cancer.
